(19) United States
(12) Patent Application Publication
(10) Pub. No.: US 2011/0033946 A1
(43) Pub. Date: Feb. 10, 2011

(54) DETECTION OF SHORT-CHAIN FATTY ACIDS IN BIOLOGICAL SAMPLES

(75) Inventors: Ronald J. Berenson, Mercer Island, WA (US); Patrick Bobbitt, Seattle, WA (US); Zhongping Lin, Wilimington, DE (US)

Correspondence Address: WILSON, SONSINI, GOODRICH & ROSATI, 650 PAGE MILL ROAD, PALO ALTO, CA, 94304-1050, US

(73) Assignee: HEMAQUEST PHARMACEUTICALS, INC., Seattle, WA (US)

(21) Appl. No.: 12/834,720

(22) Filed: Jul. 12, 2010

Related U.S. Application Data
(63) Continuation-in-part of application No. PCT/US10/27063, filed on Mar. 11, 2010.
(60) Provisional application No. 61/159,308, filed on Mar. 11, 2009.

(51) Int. Cl. G01N 33/50

(52) U.S. Cl. 436/129

(57) ABSTRACT

Described herein is a method of detecting and/or quantifying analytes, such as short-chain fatty acids. Analysis for the presence and/or quantity of the small molecule can be performed on a biological sample from a subject. In some embodiments, a liquid chromatography/mass spectrometry (LC-MS/MS) instrumentation is combined with a solid-phase extraction (SPE). Methods of derivatization can also be incorporated with LC-MS/MS and SPE instrumentation to detect and quantify target analytes. In addition to derivation, methods of reconstituting derivatized molecules can also be incorporated with LC-MS/MS and SPE instrumentation to detect and quantify target analytes.

### CROSS-REFERENCE

This application is filed under 35 U.S.C. §111(a) as a Continuation-in-Part of PCT/US10/27063 filed Mar. 11, 2010, which claims the benefit of U.S. Provisional Application No. 61/159,308, filed Mar. 11, 2009, which application is incorporated herein by reference.

### BACKGROUND OF THE INVENTION

Liquid chromatography-tandem mass spectrometry (LC-MS/MS) is a technique useful for analysis of small molecules such as pharmaceuticals as well as biological molecules such as peptides or carbohydrates. LC-MS/MS takes advantage of the benefits of both liquid chromatography and mass spectrometry by combining the two techniques. Generally, in tandem mass analysis, molecules produced from the first round of mass spectrometry are further analyzed in the second mass spectrometry.

Although LC-MS/MS is sensitive and fast, quantitative techniques for analysis of some smaller molecular compounds yield a low response in either the positive or negative ionization mode of LC-MS/MS. Disclosed herein are methodologies allowing for the quantitative analysis of small molecule compounds, such as short chain fatty acids.

### SUMMARY OF THE INVENTION

Disclosed herein is a method for detecting and/or quantifying a short-chain fatty acid in a biological sample from a subject, comprising: purifying the short-chain fatty acid by removing at least a portion of non-short-chain fatty acid components of the sample, wherein the purifying step comprises subjecting the sample to solid phase extraction; chemically derivatizing the short-chain fatty acid; subjecting said derivatized product to mass spectrometry; and determining the presence or quantity of the derivatized product, thereby detecting and/or quantifying said short-chain fatty acid in said sample. The short-chain fatty acid detected can be butyric acid or a derivative or metabolite of butyric acid, for example 2,2-dimethylbutyric acid. The biological sample can be from a human. In some embodiments, the subject has received a therapeutic dose of 2,2-dimethylbutyric acid or a pharmaceutically acceptable salt thereof. The biological sample can be a blood or urine sample. In practicing the methods herein, a short-chain fatty acid can be derivatized using a fluorinating agent, an aromatic amine, or both. Additionally, methods described herein can comprise an additional step of reconstituting the derivatized product prior to subjecting the derivatized product to mass spectrometry. Reconstitution can comprise exposing the derivatized product to a mixture of water and acetonitrile, for example a mixture where the water and acetonitrile are at a ratio of at least 75/25 v/v. In some instances, the short-chain fatty acid is a therapeutic short-chain fatty acid.

Also described herein is a method of monitoring treatment in a subject receiving a therapeutic short-chain fatty acid, comprising: purifying a short-chain fatty acid from the subject, wherein the purifying comprises subjecting the sample to solid phase extraction; chemically derivatizing the purified short-chain fatty acid; subjecting the derivatized product to mass spectrometry; determining the quantity of the therapeutic short-chain fatty acid in the sample; and using the data collected to make a clinical decision. The therapeutic short-chain fatty acid assayed for can be butyric acid or a butyric acid derivative or metabolite, or a pharmaceutically acceptable salt or ester thereof, for example, 2,2-dimethylbutyrate or a pharmaceutically acceptable salt or ester thereof. The sample can be collected from a human. In some instances, the subject has, or is at risk of developing, a blood disorder, for example sickle cell anemia or beta thalassemia. In other instances, the subject has or is at risk of developing a cell proliferative disorder, such as cancer or cytopenia. Additionally, methods described herein can comprise an additional step of reconstituting the derivatized product prior to subjecting the derivatized product to mass spectrometry. In some instances, the subject has, or is at risk of developing, a viral related proliferative disorder, a viral related malignancy, an inflammatory disorder, an autoimmune disease and/or atherosclerosis.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1. Representative Calibration Curve (dog).

FIG. 2. Representative Chromatograms of Control Dog Plasma sodium 2,2-dimethylbutyrate (top), DMV (bottom).

FIG. 3. Representative Chromatograms of Standard-1 (0.2 μg/mL sodium 2,2-dimethylbutyrate) in Dog Plasma, Sodium 2,2-Dimethylbutyrate (top), DMV (bottom).

FIG. 4. Representative Chromatograms of LLOQ (0.2 μg/mL sodium 2,2-dimethylbutyrate) in Dog Plasma sodium 2,2-dimethylbutyrate (top), DMV (bottom).

FIG. 5. Representative Chromatograms of Low QC (0.6 μg/mL sodium 2,2-dimethylbutyrate) in Dog Plasma, sodium 2,2-dimethylbutyrate (top), DMV (bottom).

FIG. 6. Representative Chromatograms of QC-Mid (10 μg/mL sodium 2,2-dimethylbutyrate) in Dog Plasma sodium 2,2-dimethylbutyrate (top), DMV (bottom).

FIG. 7. Representative Chromatograms of QC-High (40 μg/mL sodium 2,2-dimethylbutyrate) in Dog Plasma sodium 2,2-dimethylbutyrate (top), DMV (bottom).

FIG. 8. Representative Calibration Curve (human).

FIG. 9. Representative Chromatograms of Control Human Plasma sodium 2,2-dimethylbutyrate (top), DMV (bottom).

FIG. 10. Representative Chromatograms of Standard-1 (0.2 μg/mL sodium 2,2-dimethylbutyrate) in Human Plasma sodium 2,2-dimethylbutyrate (top), DMV (bottom).

FIG. 11. Representative Chromatograms of LLOQ (0.2 μg/mL sodium 2,2-dimethylbutyrate) in Human Plasma sodium 2,2-dimethylbutyrate (top), DMV (bottom).

FIG. 12. Representative Chromatograms of Low QC (0.6 μg/mL sodium 2,2-dimethylbutyrate) in Human Plasma sodium 2,2-dimethylbutyrate (top), DMV (bottom).

FIG. 13. Representative Chromatograms of QC-Mid (10 μg/mL sodium 2,2-dimethylbutyrate) in Human Plasma sodium 2,2-dimethylbutyrate (top), DMV (bottom).

FIG. 14. Representative Chromatograms of QC-High (40 μg/mL sodium 2,2-dimethylbutyrate) in Human Plasma sodium 2,2-dimethylbutyrate (top), DMV (bottom).

FIG. 15. Representative Calibration Curve (rat).

FIG. 16. Representative Chromatograms of Control Rat Plasma sodium 2,2-dimethylbutyrate (top), DMV (bottom).

FIG. 17. Representative Chromatograms of Standard-1 (0.2 μg/mL sodium 2,2-dimethylbutyrate) in Rat Plasma, sodium 2,2-dimethylbutyrate (top), DMV (bottom).

FIG. 18. Representative Chromatograms of LLOQ (0.2 μg/mL sodium 2,2-dimethylbutyrate) in Rat Plasma sodium 2,2-dimethylbutyrate (top), DMV (bottom).

FIG. 19. Representative Chromatograms of Low QC (0.6 μg/mL sodium 2,2-dimethylbutyrate) in Rat Plasma sodium 2,2-dimethylbutyrate (top), DMV (bottom).

FIG. 20. Representative Chromatograms of QC-Mid (10 μg/mL sodium 2,2-dimethylbutyrate) in Rat Plasma sodium 2,2-dimethylbutyrate (top), DMV (bottom).

FIG. 21. Representative Chromatograms of QC-High (40 μg/mL sodium 2,2-dimethylbutyrate) in Rat Plasma, sodium 2,2-dimethylbutyrate (top), DMV (bottom).

FIG. 22. Representative Chromatogram for sodium 2,2-dimethylbutyrate (LLOQ). LLOQ is established to detect butyric acid or a butyric acid derivative or metabolite by extrapolating unknown quantities of butyric acid or a derivative or metabolite in a test sample from a standard curve formed by low, mid and high QC.

FIG. 23. Representative chromatogram of sodium 2,2-dimethylbutyrate in blank human urine.

FIG. 24. Representative chromatogram of lowest calibration standard for measuring sodium 2,2-dimethylbutyrate in human urine.

FIG. 25. Representative chromatogram of mid-range quality control sample for measuring sodium 2,2-dimethylbutyrate in human urine.

FIG. 26. Representative calibration curve for measuring sodium 2,2-dimethylbutyrate in human urine.

### DETAILED DESCRIPTION OF THE INVENTION

Described herein is a method of detecting and/or quantifying small molecules, such as short-chain fatty acids in a biological sample from a subject. As described herein, an LC-MS/MS instrumentation is combined with a solid-phase extraction (SPE). Methods of derivatization can also be incorporated with LC-MS/MS and SPE instrumentation to detect and quantify the small molecules. In addition to derivation, methods of reconstituting derivatized molecules are also incorporated with LC-MS/MS and SPE instrumentation to detect and quantify short-chain fatty acid. Further disclosed herein are compositions allowing for the extraction, derivatization and analysis of particular small molecules, for example 2,2-dimethylbutyrate.

Methods described herein can be used for the analysis of molecules such as short-chain fatty acids. Typically the methods are useful for short-chain fatty acids which are difficult to detect or quantitate by available means. The methods described herein can be used to detect the presence or level of a short-chain fatty acid in any sample, for example, a biological sample (blood or plasma samples), pharmaceutical samples (e.g., batches of therapeutic short-chain fatty acids), etc. The short-chain fatty acid may be difficult to detect due to interfering substances within the sample. As described below the present disclosure provides a novel way of detecting the short-chain fatty acids.

Analysis can be performed on a sample, such as a urine sample or blood (plasma) sample to determine the presence, absence or amount of a target short-chain fatty acid. Thus, the methods described herein can provide for qualitative analysis, quantitative analysis or both. Often, biological samples contain substances (e.g., lipids, proteins, carbohydrates, etc.) or cells (or components thereof) which could interfere with analysis. Therefore, the sample can be purified or partially purified prior to analysis. Such purification can entail subjecting the sample to a SPE device (e.g., an Oasis HLB SPE cartridge) which binds or attracts the short-chain fatty acid. The short-chain fatty acid binds to the SPE device and other components of the sample are removed, for example by washing with an appropriate substance (e.g., a mixture of water and acetonitrile). Purifying or partial purifying refers to the removal of any substance which is not the target analyte from the sample, such that 50-100% of all non-analytes in the sample are removed, for example, 50%, 55%, 60%, 65%, 70%, 75%, 80%, 85%, 90%, 95% or more. In some instances, purifying or partial purifying also refers to removal of water from the sample. In instances where all or most of the water is removed, the purified short-chain fatty acid can be reconstituted, for example by adding a mixture of water and acetonitrile to the SPE.

Once a short-chain fatty acid is purified from a sample, it can be chemically modified, or derivatized. For example, if the analyte is 2,2-dimethylbutyric acid, the acid can be treated with Deoxo-fluor which converts the carboxylic acid group to an ester. Typically, derivatization is utilized to enhance detection of the target short-chain fatty acid by converting it to a chemical form which is more easily, readily, and/or accurately detected by mass spectrometry. Following derivatization, the derivitized product is then subjected to a process for detection, such as HPLC, mass spectrometry, or a combination thereof, for example HPLC followed by tandem mass spectrometry. The resulting data provide quantitative and/or qualitative data regarding the amount and/or presence of the derivatized product, which is then used to determine the amount and/or presence of the target short-chain fatty acid in the sample.

Where the starting sample is a biological sample such as a blood, plasma or urine sample from a patient, the data collected can be used to determine the level of the target short-chain fatty acid in that sample. Such an approach can be useful in determining a therapeutic regimen where the short-chain fatty acid is provided to the patient as a therapeutic agent for a disorder. For example, determining the plasma level of 2,2-dimethylbutyrate in a patient receiving the short-chain fatty acid for therapy to treat beta thalassemia, can provide a physician or other medical professional important information regarding the short-chain fatty acid's pharmacokinetics in that individual. In some instances, for example, where the patient exhibits plasma levels in a toxic range for 2,2-dimethylbutyrate, a physician may decide to lower the dosing regimen. Conversely, if the plasma level of 2,2-dimethylbutyrate is low, then a dosing regimen can be increased.

### DEFINITIONS

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g. 1 to 6.1, and ending with a maximum value of 10 or less, e.g., 5.5 to 10.

The terms “a”, “an”, and “the” refer to “one or more” when used in this application, including the claims. Thus, for example, reference to “a cell” includes a plurality of such cells, unless the context clearly is to the contrary (e.g., a plurality of cells), and so forth.

As used herein, the terms “purify” or “separate” or derivations thereof do not necessarily refer to the removal of all materials other than the analyte(s) of interest from a sample matrix. Instead, in some embodiments, the terms “purify” or “separate” refer to a procedure that enriches the amount of one or more analytes of interest relative to one or more other components present in the sample matrix. In some embodiments, a “purification” or “separation” procedure can be used to remove one or more components of a sample that could interfere with the detection of the analyte, for example, one or more components that could interfere with detection of an analyte by mass spectrometry.

As used herein, “derivatizing” means reacting two molecules to form a new molecule.

As used herein, “chromatography” refers to a process in which a chemical mixture carried by a liquid or gas is separated into components as a result of differential distribution of the chemical entities as they flow around or over a stationary liquid or solid phase.

As used herein, “liquid chromatography” (LC) means a process of selective retardation of one or more components of a fluid solution as the fluid uniformly percolates through a column of a finely divided substance, or through capillary passageways. The retardation results from the distribution of the components of the mixture between one or more stationary phases and the bulk fluid, (i.e., mobile phase), as this fluid moves relative to the stationary phase(s). “Liquid chromatography” includes reverse phase liquid chromatography (RPLC), high performance liquid chromatography (HPLC) and high turbulence liquid chromatography (HTLC).

As used herein, the term “HPLC” or “high performance liquid chromatography” refers to liquid chromatography in which the degree of separation is increased by forcing the mobile phase under pressure through a stationary phase, typically a densely packed column. The chromatographic column typically includes a medium (i.e., a packing material) to facilitate separation of chemical moieties (i.e., fractionation). The medium may include minute particles. The particles include a bonded surface that interacts with the various chemical moieties to facilitate separation of the chemical moieties such as the biomarker analytes quantified in the experiments herein. One suitable bonded surface is a hydrophobic bonded surface such as an alkyl bonded surface. Alkyl bonded surfaces may include C-4, C-8, or C-18 bonded alkyl groups, preferably C-18 bonded groups. The chromatographic column includes an inlet port for receiving a sample and an outlet port for discharging an effluent that includes the fractionated sample. In the method, the sample (or pre-purified sample) may be applied to the column at the inlet port, eluted with a solvent or solvent mixture, and discharged at the outlet port. Different solvent modes may be selected for eluting different analytes of interest. For example, liquid chromatography may be performed using a gradient mode, an isocratic mode, or a polytyptic (i.e. mixed) mode. In one embodiment, HPLC may performed on a multiplexed analytical HPLC system with a C18 solid phase using isocratic separation with water:methanol or water:acetonitrile as the mobile phase.

As used herein, the term column refers to a chromatography column having sufficient chromatographic plates to effect a separation of the components of a test sample matrix. Preferably, the components eluted from the analytical column are separated in such a way to allow the presence or amount of an analyte(s) of interest to be determined. In some embodiments, the analytical column comprises particles having an average diameter of about 5 μm. In some embodiments, the analytical column is a functionalized silica or polymer-silica hybrid, or a polymeric particle or monolithic silica stationary phase, such as a phenyl-hexyl functionalized analytical column.

The term “electron ionization” as used herein refers to methods in which an analyte of interest in a gaseous or vapor phase interacts with a flow of electrons. Impact of the electrons with the analyte produces analyte ions, which may then be subjected to a mass spectrometry technique.

The term “chemical ionization” as used herein refers to methods in which a reagent gas (e.g. ammonia) is subjected to electron impact, and analyte ions are formed by the interaction of reagent gas ions and analyte molecules.

The term “field desorption” as used herein refers to methods in which a non-volatile test sample is placed on an ionization surface, and an intense electric field is used to generate analyte ions.

The term “matrix-assisted laser desorption ionization,” or “MALDI” as used herein refers to methods in which a non-volatile sample is exposed to laser irradiation, which desorbs and ionizes analytes in the sample by various ionization pathways, including photo-ionization, protonation, deprotonation, and cluster decay. For MALDI, the sample is mixed with an energy-absorbing matrix, which facilitates desorption of analyte molecules.

The term “surface enhanced laser desorption ionization,” or “SELDI” as used herein refers to another method in which a non-volatile sample is exposed to laser irradiation, which desorbs and ionizes analytes in the sample by various ionization pathways, including photo-ionization, protonation, deprotonation, and cluster decay. For SELDI, the sample is typically bound to a surface that preferentially retains one or more analytes of interest. As in MALDI, this process may also employ an energy-absorbing material to facilitate ionization.

The term “electrospray ionization,” or “ESI,” as used herein refers to methods in which a solution is passed along a short length of capillary tube, to the end of which is applied a high positive or negative electric potential. Upon reaching the end of the tube, the solution may be vaporized (nebulized) into a jet or spray of very small droplets of solution in solvent vapor. This mist of droplet can flow through an evaporation chamber which is heated slightly to prevent condensation and to evaporate solvent. As the droplets get smaller the electrical surface charge density increases until such time that the natural repulsion between like charges causes ions as well as neutral molecules to be released.

The term “Atmospheric Pressure Chemical Ionization,” or “APCI,” as used herein refers to mass spectroscopy methods that are similar to ESI, however, APCI produces ions by ion-molecule reactions that occur within a plasma at atmospheric pressure. The plasma is maintained by an electric discharge between the spray capillary and a counter electrode. Then, ions are typically extracted into a mass analyzer by use of a set of differentially pumped skimmer stages. A counterflow of dry and preheated N2 gas may be used to improve removal of solvent. The gas-phase ionization in APCI can be more effective than ESI for analyzing less-polar species.

The term “inductively coupled plasma” as used herein refers to methods in which a sample is interacted with a partially ionized gas at a sufficiently high temperature to atomize and ionize most elements.

The term “ionization” and “ionizing” as used herein refers to the process of generating an analyte ion having a net electrical charge equal to one or more electron units. Negative ions are those ions having a net negative charge of one or more electron units, while positive ions are those ions having a net positive charge of one or more electron units.

The term “desorption” as used herein refers to the removal of an analyte from a surface and/or the entry of an analyte into a gaseous phase.

LC-MS Instrumentation

Provided herein are methods of detecting and quantitating fatty acid. In one embodiment, detection and quantitation is accomplished by the use of LC and MS. As disclosed herein, an LC instrument and an MS instrument can be set up in a particular way to achieve the detection and quantitation of a fatty acid molecule. In one embodiment, a set up can be LC-MS. In another embodiment, a set up can be LC-MS/MS in which two mass analyzers are operably connected in a tandem fashion. Operable connection can be a physical connection with a vacuum chamber connecting the two MS into a one, continuously connected unit. Operable connection can also be two separate mass analyzers located in close proximity in which a sample from first analyzer can continuously be transferred to the second mass analyzer. Continuous transfer can be either done by an automated process or by a manual process. An automated process can be a mechanical process controlled by a computer-readable logic commands.

High performance liquid chromatography (also known as high pressure liquid chromatography or HPLC) can be utilized as an LC approach that is combined with an MS technique. HPLC can be used to separate, identify, and quantify compounds based on their chemical properties such as polarities and interactions with the stationary phase of a column. Depending on characteristics of the stationary phase of an HPLC, such as hydrophobicity, polarity, or enantiomeric properties, molecules passing through the stationary phases are separated by their ability to interact with the stationary phase and/or the strength of the interaction. To facilitate the movement of analytes through the stationary phase, a high pressure pump is utilized. Molecules coming out of HPLC column are monitored by a spectroscopic device such as an ultraviolet or visible spectrometer.

Tandem mass spectrometry involves multiple steps of mass spectrometry in which each step of spectrometry can be designed to select certain types of molecules. The selection can be performed based on characteristics of the substance(s) to be analyzed and/or assayed for, for example, selection can be performed by molecular weight of a target molecule. Depending on the types of mass spectrometry, MS/MS can involve some form of fragmentation occurring in between the stages.

Separation of target molecules from a sample typically depends on physical characteristics of the molecule, such as sectors, transmission quadrupole, or time-of-flight. Molecules that are ionized and trapped in the first mass spectrometer are analyzed in the second mass spectrometer. By performing tandem mass spectrometry in time, the separation is accomplished with ions trapped in the same place, with multiple separation steps taking place over time. A quadrupole ion trap or FTMS instrument can be used for such an analysis.

A number of different MS/MS experiments have been used to date. MS/MS tandem analysis can be done in either time or space. MS/MS in space involves the physical separation of the instrument components. MS/MS in time involves the use of an ion trap.

In one approach, a precursor ion scan method, a product ion is selected in the second mass analyzer, and the precursor masses are scanned in the first mass analyzer. In another approach, a product ion scan method, a precursor ion is selected in the first stage, allowed to fragment and then all, some or most, resulting masses are scanned in the second mass analyzer and detected in a detector positioned after the second mass analyzer. In still another approach, a neutral loss scan method, the first mass analyzer scans all the masses. The second mass analyzer also scans, but scans a set offset from the first mass analyzer. This offset corresponds to a neutral loss that is commonly observed for the class of compounds. In another approach, a selected reaction monitoring method, both mass analyzers are set to a selected mass.

In some instances, fragmentation of the molecule(s) of interest is required. For example, if the molecule of interest has a molecular weight greater than the limit a mass analyzer can resolve, fragmentation is utilized. When fragmentation is used, fragmentation of gas-phase ions usually occurs between different stages of mass analysis. Many different methods are known in the art for the fragmentation of ions. In-source fragmentation refers to a method in which the ionization process of a mass analyzer causes fragmentation of a molecule in mass spectrometer. In-source fragmentation occurs when the ionization energy imparted on the molecule is at a sufficient level to fragment the molecule into smaller pieces. Post-source fragmentation is another approach in which the molecule is purposefully fragmented. Post-source fragmentation is frequently used in a MS/MS system. Energy can also be added to the ions through post-source collisions with neutral atoms or molecules, the absorption of radiation, or the transfer or capture of an electron by a multiply charged ion.

A LC instrument useful for methods described herein includes, but is not limited to, an HPLC, affinity chromatography, size exclusion chromatography, reversed-phase chromatography, two-dimensional chromatography, chiral chromatography, countercurrent chromatography, fast protein liquid chromatography, simulated moving-bed chromatography, and ion-exchange chromatography. Gas chromatography can also be useful for methods described herein. In one embodiment, an HPLC comprises an instrument for detecting and quantitating a molecule of interest, such as short-chain fatty acid or a derivatized product thereof. For example, an HPLC used for the methodology herein may be specifically designed to detect 2,2-dimethylbutyrate or an esterated derivative or metabolite.

A MS instrument useful for methods described herein can utilize various ionization techniques. Useful ionization technique include, but are not limited to, electrospray ionization and matrix-assisted laser desorption/ionization, inductively coupled plasma (ICP), glow discharge, field desorption (FD), fast atom bombardment (FAB), thermospray, desorption/ionization on silicon (DIOS), direct analysis in real time (DART), atmospheric pressure chemical ionization (APCI), secondary ion mass spectrometry (SIMS), spark ionization and thermal ionization (TIMS), and ion attachment ionization.

A MS instrument useful for methods described herein can utilize various mass analysis techniques. A useful mass analyzer can include multiple capabilities, for example, a sector field mass analyzer, a time-of-flight mass analyzer, a quadrupole mass analyzer, a quadrupole ion trap, a linear quadrupole ion trap, a Fourier transform ion cyclotron resonance mass analyzer, an orbitrap, an ion cyclotron resonance mass analyzer, or any combination of these.

A MS instrument useful for methods described herein can utilize various fragmentation techniques. Fragmentation techniques include, but are not limited to, collision-induced dissociation (CID), electron capture dissociation (ECD), electron transfer dissociation (ETD), infrared multiphoton dissociation (IRMPD) and blackbody infrared radiative dissociation (BIRD). MS instrumentalities can also include multiple configurations, such as tandem mass spectrometry (MS/MS), a matrix-assisted laser desorption/ionization with a time-of-flight mass analyzer (MALDI-TOF), SELDI, inductively coupled plasma-mass spectrometry (ICP-MS), accelerator mass spectrometry (AMS), Thermal ionization-mass spectrometry (TIMS), spark source mass spectrometry (SSMS), and isotope ratio mass spectrometry (IRMS).

Solid Phase Extraction

Solid-phase extraction (SPE) is a separation process. A sample dissolved or suspended in a liquid mixture forms a mobile phase. A stationary phase is present within a columnar or other appropriately configured structure. The mobile phase is flown over a stationary phase. Molecular interactions between the molecules in mobile phase and the stationary phase lead to a separation of molecules in the sample.

Depending on the properties of stationary phase, a mixture of molecules can be separated and concentrated according to the physical characteristics of each component of the mixture. In some instances, analytes retained within a column due to interaction (e.g., attachment or attraction) with the stationary phase, are eluted with another molecule that competitively binds to the stationary phase, resulting in the elution of the analytes.

A solid-phase extractions useful for detecting and quantitating small molecules such as short-chain fatty acids includes, but are not limited to, a normal phase SPE in which a molecule of interest is retained in the column while unwanted molecules are washed out, and then later eluted with a solvent that disrupts the interaction; a reversed-phase SPE in which the stationary phase comprises derivatized material containing one or more hydrocarbons and a reversed-phase SPE anion-exchanger such as a cation exchanger or an anion exchanger.

Silica can be used to form part or all of the solid phase of an extraction component. Silica packed into a syringe can form a porous body into which an analyte can pass through. Silica used for forming a solid phase can be derivatized. Additionally, silica particles can be derivatized to present functions groups such as an octyl group or an octadecyl group. SPEs can also be hydrophobic and/or contain specific reactive groups, such as octyl groups or ocadecyl groups.

Derivatization

Derivatization is a term used for the transformation of one chemical compound into a product of similar chemical structure (i.e., a “chemical derivative”). “Chemical derivatives” refers to products produced by the exposure of a target molecule (e.g., a short-chain fatty acid) to a derivatizing agent Generally, one or more specific functional groups of the target compound or molecule (i.e., the educt) are transformed through one or more chemical reactions to produce the chemical derivative. The production of a chemical derivative in the methods disclosed herein is useful where the target compound or molecule is difficult to detect and/or quantitate in unmodified form. A useful chemical derivative will typically differ in one or more chemical characteristics, including but not limited to, reactivity, solubility, aggregate state, chemical composition, boiling point or melting point. A chemical derivative is typically easier to detect and/or quantitate using the methods described herein and can, therefore, be used to detect and/or quantitate the target compound or molecule in a sample. Derivatization reactions useful in practicing the methods described herein typically proceed to completion if quantitation of the target is desired. Derivatization reactions can be general reactions which affect multiple substrates, but are specific to one or more chemical groups targeted. Typically, a chemical derivative product is relatively chemically stable, allowing sufficient time for detection and/or quantitation of the chemical derivative.

An agent used to form a chemical derivative from an educt (i.e., a derivatizing agent) can be any agent appropriate to produce a desired chemical change in a target compound or molecule. Exemplary derivatizing agents include, but are not limited to, isothiocyanate groups, dansyl groups, dinitro-fluorophenyl groups, nitrophenoxycarbonyl groups, phthalaldehyde groups, alkylating agents, methylating agents such as methanolic hydrogen chloride, activated imidazole compounds such as 2-methoxy-4,5 dihydro 1H-imidazole, buffers, solvents, fluorinated phosphazines, polyethylene glycols, alkyl amines and fluorinated carboxylic acids. Derivatizing agents can be used alone, or in combination, to produce the desired chemical derivative. For example, a chemical derivatization is accomplished using fluorinating agent and an aromatic amine.

In one aspect, derivatization is performed to promote desorption and ionization of analyte. In one embodiment, derivatization is to modify an analyte in a sample to form a bound complex with a presentation apparatus of a mass spectrometer. A derivatized sample presentation apparatus can be composed of any suitable material. The material can be a solid or liquid. Suitable solid materials include, but are not limited to insulators such as quartz, semiconductors such as doped silicon and the like, and conductors including metals such as steel, gold and the like. Various insulating or conductive polymers may also be used. The surface of the sample presentation apparatus need not be made of the same material as the rest of the apparatus. It is preferable for the surface to be clean so that a complex may adhere to the surface.

Derivation can include tethering in which a molecular tether is used to form a complex that binds to the sample presentation apparatus. A tethering molecule includes, but is not limited to, dithiothreitol, dimethyladipimidate-2*HCL, dimethylpimelimidate*HCL, dimethylsuberimidate*2HCL, dimethyl 3,3′-dithiobispropionimidate*2HCL, disuccinimidyl glutarate, disuccinimidyl suberate, bis(sulfosuccinimidyl)suberate, dithiobis(succinimidylpropionate), dithiobis(sulfosuccinimidylpropionate), ethylene glycobis (succinimidylsuccinate), ethylene glycobis(sulfosuccinimidylsuccinate), disuccinimidyl tartarate, disulfosuccinimidyl tartarate, bis[2-(succinimidyloxycarbonyloxy)ethyl]sulfone, bis[2-(sulfosuccinimidooxy-carbonyloxy)ethyl]sulfone, succinimidyl 4-(N-maleimido-matyl) cyclohexane-1-carboxylate, sulfo-succinimidyl4-(N-maleimidomethyl)cyclohexane-1-carboxylare, m-Maleimidobenzoyl-N-hydroxysuccinimide ester, m-maleimidobenzoyl-N-hydroxysulfosuccinimide ester, succinimidyl 4-p-maleimido-phenyl)-butyrate, sulfosuccinimidyl 4-(p-maleimidophenyl)-butyrate, bismaleimidohexane, N-(λ-maleimidobutyryloxy)succinimide ester, N-(λ-maleimidobutyryloxy)sulfosuccinimide ester, n-succinimidyl(4-iodoacetyl)aminobenzoate, sulfosuccinimidyl(4-iodoacetyl)-aminobenzoate, 1,4-di-[3′-2′-pyridyldithio(propionamido)butane], 4-succinimidyl-oxycarbonyl-α(s-pyridyldithio)toluene, sulfosuccinimidyl-6-[α-methyl-α-(2-pyridyldithio)-toluamido]hexanoate, n-succinimidyl-3(2-pyridyldithio)-propionate, succinimidyl 6-[3-(2-pyridyldithio)-propionamido]hexanoate, sulfosuccinimidyl-6-[-3-(2-pyridyldithio)-propionamido]hexanoate, sulfosuccinimidyl-6-[3-(2-pyridyldithio)-propionamido]hexanoate, 3-(2-pyridyldithio)-propionyl hydrazine, 1-ethyl-3-(3-dimethylaminopropyl)-carbodiimide hydrochloride, n,n′-dicyclohexylcarbodiimide, 4-(p-azidosalicylamido)-butylamine, azidobenzoyl hydrazine, N-5-azido-2-nitrobenzoyloxysuccinimide, n-5-azido-2-nitrobenzoyloxysuccinimide, n-[4-(p-azidosalicylamido)butyl]-3′(2′-pyridyldithio)propionamide, p-azidophenyl glyoxal monohydrate, 4-(p-azidosalicyl-amido)butylamine, 1-(p-azidosalicylamido)-4-(iodoacetamido)butane, bis-[β-4-azidosalicylamido)ethyl]disulfide, n-hydroxysuccinimidyl-4-azidobenzoate, n-hydroxysulfo-succinimidyl 4-azidobenzoate, n-hydroxysuccinimidyl-4-azidosalicylic acid, n-hydroxysulfosuccinimidyl-4-azidosalicylic acid, sulfosuccinimidyl-(4-azidosalicylamido)-hexanoate, p-nitrophenyl-2-diazo-3,3,3-trifluoropropionate, 2-diazo-3,3,3,-trifluoro-propionate, n-succinimidyl-(4-azidophenyl)1,3/-dithiopropionate, sulfosuccinimidyl-(4-azidophenyldithio)propionate, sulfosuccinimidyl-2-(7-azido-4-methylcoumarin-3-acetoamide)ethyl-1,3′-dithiopropionate, sulfosuccinimidyl 7-azido-4-methylcoumarin-3-acetate, sulfosuccinimidyl 2-(m-azido-o-nitrobenzamido)-ethyl-1,3′-dithiopropionate, n-succeinimidyl-6-(4′-azido-2′-nitrophenyl-amino)hexanoate, sulfosuccinimidyl 6-(4′-azido-2′-nitrophenylamino)hexanoate, sulfosuccinimidyl 2-(p-azidosalicylamido)ethyl-1,3′-dithiopropionate, and sulfosuccinimidyl 4-(p-azidophenyl)-butyrate.

Reconstitution

A reconstitution buffer suitable for reconstituting a derivatized fatty acid includes, but is not limited to buffers containing acetonitrile, trifluoroacetic acid, tetrahydrofuran, trimethyleneamine, triethylammonium bicarbonate, methanol, alpha-cyano-4-hydroxycinnamic acid (CHCA), formic acid, water, and biological buffers such as Tris-based buffers and phosphate-based buffers. Any single buffer can contain any combination of these components in any amount, as appropriate to the individual derivatized product to be analyzed. The precise buffer used can be altered as necessary for reconstituting a particular derivatized fatty acid and/or for compatibility with the HPLC and/or mass spectrometry analytical design/instrumentation utilized.

Fatty Acids

The methods described herein can be used to detect fatty acids, and in particular, short-chain fatty acids. Short-chain fatty acids are fatty acids typically with aliphatic tails of six carbons or less. A short chain fatty acid includes, but is not limited to, C3-C12 fatty acids, C3-C10 fatty acids, C3-C8 fatty acids, methoxyacetic acid, butyric acid (BA), valproic acid (VPA), propionic acid, 3-methoxypropionic acid, and ethoxyacetic acid. As used herein, the term short-chain fatty acid also refers to salts or esters of fatty acids, especially pharmaceutically acceptable salts and esters of fatty acids (e.g., sodium butyrate, arginine butyrate). Additionally, the term short-chain fatty acid also refers to “derivatives” of fatty acids, such as fatty acids containing substitutions at one or more positions (e.g., sodium 2,2-dimethylbutyric acid, α-amino-n-butyrate). This use of the term derivatives is distinguished from “chemical derivatives” as used herein as “chemical derivatives” refers to those products produced by the use of a derivatizing agent on a target molecule (e.g., a short-chain fatty acid). In one embodiment of a method of the invention, the short-chain fatty acid is 2,2-dimethylbutyric acid or pharmaceutically acceptable salts thereof.

A short-chain fatty can be a naturally occurring in a subject or can be a short chain fatty acid that is administered to a subject to treat a disorder, including but not limited to, a cancer, a blood disorder, or a cell proliferative disorder. A fatty acid assayed for can be specific to a particular anatomical location, or exhibit generalized distribution throughout the body of a subject. Naturally occurring includes fermentation of food product by a microorganism which is a commensal or a pathogen. Short chain fatty acids include, but are not limited to, formic, acetic, propionic, butyric, isobutyric, pentanoic, isopentenoic, and caproic acid. A short-chain fatty acid can be a product of hydrolysis of glycerides, such as a tirglyceride, diglyceride, or monoglyceride.

In one aspect, methods disclosed herein are useful to detect and quantitate pharmaceutically useful short-chain fatty acids, and/or acceptable salts thereof. For example, butyric acid and multiple derivatives or metabolites of butyric acid have been shown to be useful in treating a wide variety of disorders, including cystic fibrosis, blood disorders (e.g., sickle cell disease and beta thalassemia) and cell proliferative disorders. See, e.g., U.S. Pat. Nos. 5,939,456; 6,011,000; 6,231,880; 6,677,302; 7,265,153; PCT International App. No. PCT/US94/11565; Perrine et al., (1987) Biochem. Biophys. Res. Comm., 148(2): 694-700 (each of which is incorporated by reference for all purposes).

In one embodiment, a fatty acid is a short chain fatty acid. Non-limiting examples of short-chain fatty acids which can be used for therapeutic purposes include, α-amino-n-butryic acid, 2,2-dimethylbutyric acid, and isobutyramide. A pharmaceutically acceptable salt includes, sodium, potassium, alkaline earth salts such as calcium magnesium ammonium salts such as trimethylammonium, and amino acid salts such as arginine.

In one embodiment, a short-chain fatty acid is acetic acid. In another embodiment, a short-chain fatty acid is propionic acid. In another embodiment, a short-chain fatty acid is butyric acid. In another embodiment, a short-chain fatty acid is isovaleric acid. In another embodiment, a short-chain fatty acid is valeric acid. In another embodiment, a short-chain fatty acid is caproic acid.

Sample, Subject, Medical Condition

The methods disclosed herein can be used to detect the level of a short-chain fatty acid that is supplied to a subject as a therapeutic agent. For example, 2,2-dimethylbutyric acid, or a pharmaceutically acceptable salt or ester thereof, can be used to treat a subject suffering from a blood disorder, such as sickle cell anemia. To determine if the patient is receiving an effective dose, the methods described herein can be used to detect the concentration of 2,2-dimethylbutyric acid. As shown in the examples, the methods described herein are sensitive and reliable. Thus, in one embodiment, the results can be utilized to assist a health care professional (e.g., a doctor) in determining an appropriate course of treatment. For example, where the results show that the subject receiving the 2,2-dimethylbutyric acid rapidly clears the substance from his or her body, a physician can increase the dosage and/or switch to a new pharmacological treatment. Data on the in vivo levels of the detected short-chain fatty acid can also be used in conjunction with other parameters to alter a therapeutic regimen of the short-chain fatty acid, for example, increasing, decreasing or maintaining a dosage regimen.

Measuring the levels of a pharmaceutical compound in a sample (e.g., plasma, or urine) can be used to determine whether the subject receiving treatment with a short chain fatty acid (e.g., DMB or a butyric acid salt) is achieving and/or maintaining a pharmaceutically acceptable level of the compound. For example, effective doses of 2,2-dimethylbutyric acid can result in blood concentrations of between 0.2 μM to more than 1000 μM and optimal ranges can include 200 μM to 800 μM or 400 μM to 600 μM. To ascertain whether such ranges are being achieved with a given patient regimen, samples of blood, plasma and/or urine can be obtained from a patient undergoing therapy. Samples can be taken 30 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, or more after dosing. Additionally, samples at one or more of these times can be collected to determine the level of a therapeutic short-chain fatty acid (e.g., DMB) at multiple time points. By determining the level of the compound in the blood, plasma or urine, a physician or other medical professional can adjust dosage levels of the compound. For example, if a sample from a patient being dosed with 100 mg of DMB three times a day is analyzed and shows less than 0.2 μM concentration in the blood, dosage can be increased. Alternately, if levels of DMB are greater than 1000 μM, dosages can be decreased. Thus, monitoring the level of a short-chain fatty acid (e.g., DMB or arginine butyrate) in a patient's blood, plasma or urine can allow for fine tuning of a therapeutic regimen.

A sample from a subject can be any type of biological fluid or solid material that can be dissolved into a fluid form. In one embodiment, a sample is plasma or urine. In another embodiment, a sample is an extract prepared from a solid tissue. Extraction can be physical, chemical, or enzymatic extraction. A physical extraction can utilized centrifugation, pulverization, filtration, meshing, grinding, heating, freezing, fracturing, agitating, homogenization, and other physical methods routinely used in a laboratory. Chemical extraction can be treating with emulsifier, soap, or ionic agent such as sarcosyl or sodium lauryl sulfate, to dissociate solid tissue. Other chemical cleavage agents include, but are not limited to, cyanogen bromide, O-iodosobenzoate or O-iodosobenzoic acid, dilute hydrochloric acid, N-bromosuccinimide, sodium hydrazine, lithium aluminum hydride, hydroxylamine and 2-nitro-5-thiocyanobenzoate, Sanger's Reagent, 2,4-dinitrofluorobenzene, tetradentate Co (III) complex, and β-[Co(triethylenetetramine)-OH(H.sub.2 O)]. Enzymatic proteases are specific polypeptides which cleave polypeptides. Proteases may cleave themselves by a process known as autolysis. Several enzymatic proteases cleave polypeptides between specific amino acid residues. Examples of proteases which cleave nonspecifically include subtilisin, papain and thermolysin. Examples of proteases which cleave at least somewhat specifically include: aminopeptidase-M, carboxypeptidase-A, carboxypeptidase-P, carboxypeptidase-B, carboxypeptidase-Y, chymotrypsin, clostripain, trypsin, elastase, endoproteinase Arg-C, endoproteinase Glu-C, endoproteinase Lys-C, factor Xa, ficin, pepsin, plasmin, staphylococcus aureus V8 protease, proteinkinase K and thrombin.

A subject includes, but is not limited to an animal, such as any mammal, including humans. A subject can be a healthy subject or a subject having a medically diagnosable condition. In another embodiment, a human is a person suspected of having a medically diagnosable condition. A medically diagnosable condition includes, but is not limited to, a cancer, an immune disorder, a hematopoietic disorder, a neurological disorder, an infectious disease, a viral-related proliferative disorder, a viral-related malignancy, an inflammatory disorder and a cardiovascular disorder, such as atherosclerosis.

In particular, short chain fatty acids can be used therapeutically to treat a number of diseases including viral-related malignancies and cell proliferative disorders, blood disorders, inflammatory diseases, autoimmune diseases, coronary diseases and some diseases of genetic origin, such as cystic fibrosis. One category of diseases which can be treated with short-chain fatty acids (e.g., butyric acid or 2,2-dimethylbutyrate) include latent viral infections including but not limited to Epstein-Barr virus (EBV), a Kaposi's-associated human herpes virus (human herpes virus 8), a human immunodeficiency virus (HIV), and a human T-cell leukemia/lymphoma virus (HTLV). Such latent viral infections can result in other diseases or cell proliferative disorders caused by, or linked to, the viral infection, for example leukemias, lymphomas, sarcomas, carcinomas, neural cell tumors, squamous cell carcinomas, germ cell tumors, undifferentiated tumors, seminomas, melanomas, neuroblastomas, mixed cell tumors, metastatic neoplasia, Burkitt's lymphoma, EBV-induced malignancies, T and B cell lymhoproliferative disorders and leukemias, and other viral-induced malignancies.

Certain short-chain fatty acids can be used to treat blood disorders, such as hemoglobinopathies (e.g., sickle cell disease and beta thalassemia). Short-chain fatty acids can also be used therapeutically to treat autoimmune diseases, whether or not associated with viral infection, including but not limited to rheumatoid arthritis, multiple sclerosis, Sjogren's syndrome, systemic lupus erythematosus, autoimmune hepatitis, autoimmune thyroiditis, hemophagocytic syndrome, diabetes, Crohn's disease, ulcerative colitis, psoriasis, psoriatic arthritis, idiopathic thrombocytonpenic pupura, polymyositis, dermatomyositis, myasthenia gravis, autoimmune thyroiditis, Evan's syndrome, autoimmune hemolytic anemia, aplastic anemia, autoimmune neutropenia, scleroderma, Reiter's syndrome, ankylosing spondylitis, pemphnigus, pemphigoid or autoimmune hepatitis. Short-chain fatty acids can also be used therapeutically to treat inflammatory diseases, including allergies, skin disorders, diseases associated with coronary artery disease or peripheral artery disease. Exemplary inflammatory diseases include retinitis, pancreatitis, cardiomyopathy, pericarditis, colitis, glomerulonephritis, lung inflammation, esophagitis, gastritis, duodenitis, ileitis, meningitis, encephalitis, encephalomyelitis, transverse myelitis, cystitis, urethritis, mucositis, lymphadenitis, dermatitis, hepatitis, osteomyelitis, or herpes zoster (shingles).

A cancer can be a carcinoma, a sarcoma, a lymphoma, a germ cell tumor, or a blastoma. A carcinoma includes, but is not limited to, epithelial neoplasms, squamous cell neoplasms squamous cell carcinoma, basal cell neoplasms basal cell carcinoma, transitional cell papillomas and carcinomas, adenomas and adenocarcinomas (glands), adenoma, adenocarcinoma, linitis plastica insulinoma, glucagonoma, gastrinoma, vipoma, cholangiocarcinoma, hepatocellular carcinoma, adenoid cystic carcinoma, carcinoid tumor of appendix, prolactinoma, oncocytoma, hurthle cell adenoma, renal cell carcinoma, grawitz tumor, multiple endocrine adenomas, endometrioid adenoma, adnexal and skin appendage neoplasms, mucoepidermoid neoplasms, cystic, mucinous and serous neoplasms, cystadenoma, pseudomyxoma peritonei, ductal, lobular and medullary neoplasms, acinar cell neoplasms, complex epithelial neoplasms, warthin's tumor, thymoma, specialized gonadal neoplasms, sex cord stromal tumor, thecoma, granulosa cell tumor, arrhenoblastoma, sertoli leydig cell tumor, glomus tumors, paraganglioma, pheochromocytoma, glomus tumor, nevi and melanomas, melanocytic nevus, malignant melanoma, melanoma, nodular melanoma, dysplastic nevus, lentigo maligna melanoma, superficial spreading melanoma, and malignant acral lentiginous melanoma. A sarcoma includes, but is not limited to, askin's tumor, botryodies, chondrosarcoma, ewing's sarcoma, malignant hemangio endothelioma, malignant schwannoma, osteosarcoma, soft tissue sarcomas including: alveolar soft part sarcoma, angiosarcoma, cystosarcoma phyllodes, dermatofibrosarcoma, desmoid tumor, desmoplastic small round cell tumor, epithelioid sarcoma, extraskeletal chondrosarcoma, extraskeletal osteosarcoma, fibrosarcoma, hemangiopericytoma, hemangiosarcoma, kaposi's sarcoma, leiomyosarcoma, liposarcoma, lymphangiosarcoma, lymphosarcoma, malignant fibrous histiocytoma, neurofibrosarcoma, rhabdomyosarcoma, and synovialsarcoma. A lymphoma includes, but is not limited to, chronic lymphocytic leukemia/small lymphocytic lymphoma, B-cell prolymphocytic leukemia, lymphoplasmacytic lymphoma (such as waldenström macroglobulinemia), splenic marginal zone lymphoma, plasma cell myeloma, plasmacytoma, monoclonal immunoglobulin deposition diseases, heavy chain diseases, extranodal marginal zone B cell lymphoma, also called malt lymphoma, nodal marginal zone B cell lymphoma (nmzl), follicular lymphoma, mantle cell lymphoma, diffuse large B cell lymphoma, mediastinal (thymic) large B cell lymphoma, intravascular large B cell lymphoma, primary effusion lymphoma, burkitt lymphoma/leukemia, T cell prolymphocytic leukemia, T cell large granular lymphocytic leukemia, aggressive NK cell leukemia, adult T cell leukemia/lymphoma, extranodal NK/T cell lymphoma, nasal type, enteropathy-type T cell lymphoma, hepatosplenic T cell lymphoma, blastic NK cell lymphoma, mycosis fungoides/sezary syndrome, primary cutaneous cd30-positive T cell lymphoproliferative disorders, primary cutaneous anaplastic large cell lymphoma, lymphomatoid papulosis, angioimmunoblastic T cell lymphoma, peripheral T cell lymphoma, unspecified, anaplastic large cell lymphoma, classical hodgkin lymphomas (nodular sclerosis, mixed cellularity, lymphocyte-rich, lymphocyte depleted or not depleted), and nodular lymphocyte-predominant hodgkin lymphoma. A germ cell tumor includes, but is not limited to, germinoma, dysgerminoma, seminoma, nongerminomatous germ cell tumor, embryonal carcinoma, endodermal sinus tumor, choriocarcinoma, teratoma, polyembryoma, and gonadoblastoma. A blastoma includes, but is not limited to, nephroblastoma, medulloblastoma, and retinoblastoma. Other cancers include, but are not limited to, labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidea melanoma, seminoma, rhabdomyosarcoma, craniopharyngeoma, osteosarcoma, chondrosarcoma, myosarcoma, liposarcoma, fibrosarcoma, Ewing sarcoma, and plasmocytoma.

A cardiovascular condition includes, but is not limited to, chronic rheumatic heart disease, hypertensive disease, ischemic heart disease, pulmonary circulatory disease, heart disease, cerebrovascular disease, diseases of arteries, arterioles and capillaries and diseases of veins and lymphatics. A chronic rheumatic heart disease includes, but is not limited to diseases of mitral valve, diseases of aortic valve, diseases of mitral and aortic valves, and diseases of other endocardial structures. A hypertensive disease includes, but is not limited to essential hypertension, hypertension, malignant, hypertension, benign, hypertension, unspecified, hypertensive heart disease, hypertensive renal disease, hypertensive renal disease, unspecified, with renal failure, hypertensive heart and renal disease, hypertension, renovascular, malignant, and hypertension, renovascular benign. An ischemic heart disease includes, but is not limited to acute myocardial infarction, myocardiac infarction, acute, anterolateral, myocardiac infarction, acute, anterior, infarction, acute, inferolateral, myocardiac infarction, acute, inferoposterior, myocardiac infarction, acute, other inferior wall, myocardiac infarction, acute, other lateral wall, myocardiac infarction, acute, true posterior, myocardiac infarction, acute, subendocardial, myocardiac infarction, acute, spec, myocardiac infarction, acute, unspecified, postmyocardial infarction syndrome, intermediate coronary syndrome, old myocardial infarction, angina pectoris, angina decubitus, prinzmetal angina, coronary atherosclerosis, aneurysm and dissection of heart, aneurysm of heart wall, aneurysm of coronary vessels, dissection of coronary artery, and unspecified chronic ischemic heart disease. A pulmonary circulatory disease includes, but is not limited to, diseases of pulmonary circulation, acute pulmonary heart disease, pulmonary embolism, not iatrogenic, chronic pulmonary heart disease, and unspecified chronic pulmonary heart disease. A heart disease includes, but is not limited to acute pericarditis, other and unspecified acute pericarditis, acute nonspecific pericarditis, acute and subacute endocarditis, acute bacterial endocarditis acute myocarditis, other and unspecified acute myocarditis, myocarditis, idiopathic, other diseases of pericardium, other diseases of endocardium, valvular disorder, mitral, valvular disorder, aortic, valvular disorder, tricuspid, valvular disorder, pulmonic, cardiomyopathy, hypertrophic obstructive cardiomyopathy, conduction disorders, atrioventricular block, third degree, atrioventricular block, first degree, atrioventricular block, mobitz, atrioventricular block, wenckebach's, bundle branch block, left, bundle branch block, right, sinoatrial heart block, atrioventricular excitation, anomalous, wolff parkinson white syndrome, cardiac dysrhythmias, tachycardia, paroxysmal supraventricular, atrial fibrillation and flutter, atrial fibrillation, atrial flutter, ventricular fibrillation and flutter, ventricular fibrillation, cardiac arrest, premature beats, unspecified, other specified cardiac dysrhythmias, sick sinus syndrome, sinus bradycardia, cardiac dysrhythmia unspecified, gallop rhythm, heart failure, heart failure, congestive, unspecified, acute pulmonary edema, systolic unspecified heart failure, acute systolic heart failure, chronic systolic heart failure, diastolic unspecified heart failure, diastolic chronic heart failure, combined unspecified heart failure, and cardiomegaly. A cerebrovascular disease includes, but is not limited to subarachnoid hemorrhage, intracerebral hemorrhage, other and unspecified intracranial hemorrhage, intracranial hemorrhage, occlusion and stenosis of precerebral arteries, occlusion and stenosis of basilar artery, occlusion and stenosis of carotid artery, occlusion and stenosis of vertebral artery, occlusion of cerebral arteries, cerebral thrombosis, cerebral thrombosis without cerebral infarction, cerebral thrombosis with cerebral infarction, cerebral embolism, cerebral embolism without cerebral infarction, cerebral embolism with cerebral infarction, transient cerebral ischemia, basilar artery syndrome, vertebral artery syndrome, subclavian steal syndrome, vertebrobasilar artery syndrome, transient ischemic attack, unspecified, acute but ill defined cerebrovascular disease, other and ill defined cerebrovascular disease, cerebral atherosclerosis, other generalized ischemic cerebrovascular disease, hypertensive encephalopathy, cerebral aneurysm nonruptured, cerebral arteritis, moyamoya disease, nonpyogenic thrombosis of intracranial venous sinus, transient global amnesia, late effects of cerebrovascular disease, cognitive deficits, speech and language deficits, unspecified speech and language deficits, aphasia, dysphasia, other speech and language deficits, hemiplegia/hemiparesis, hemiplegia affecting unspecified side, hemiplegia affecting dominant side, hemiplegia affecting nondominant side, monoplegia of upper limb, monoplegia of lower limb, other paralytic syndrome, other late effects of cerebrovascular disease, apraxia cerebrovascular disease, dysphagia cerebrovascular disease, facial weakness, ataxia, and vertigo. Diseases of arteries, arterioles and capillaries include, but are not limited to atherosclerosis, atherosclerosis of renal artery, atherosclerosis of native arteries of the extremities, intermittent claudication, atherosclerosis, extremities, without ulceration, atherosclerosis, not heart/brain, aortic aneurysm, dissection of aorta, abdominal ruptured aortic aneurysm, abdominal, without ruptured aortic aneurysm, unspecified aortic aneurysm, other aneurysm, other peripheral vascular disease, raynaud's syndrome, thromboangiitis obliterans, other arterial dissection, dissection of carotid artery, dissection of iliac artery, dissection of renal artery, dissection of vertebral artery, dissection of other artery, erythromelalgia, unspecified peripheral vascular disease, arterial embolism and thrombosis, polyarteritis nodosa and allied conditions, polyarteritis nodosa, kawasaki disease/acute febrile mucocutaneous lymph node syndrome, hypersensitivity angiitis, goodpasture's syndrome, lethal midline granuloma, wegener's granulomatosis, giant cell arteritis, thrombotic microangiopathy, takayasu's disease, other disorders of arteries and arterioles, arteriovenous fistula acquired, arteritis unspecified, vasculitis, vascular non-neoplastic nevus. Diseases of veins and lymphatics include, but are not limited to phlebitis and thrombophlebitis, femoral deep vein thrombosis, deep vein thrombosis of other leg veins, phlebitis of other sites, superficial veins of upper extremity, unspecified thrombophlebitis, portal vein thrombosis, other venous embolism and thrombosis, unspecified deep vein thrombosis, proximal deep vein thrombosis, distal deep vein thrombosis, unspecified venous embolism, varicose veins of lower extremities, varicose veins without ulcer, varicose veins without inflammation, varicose veins without ulcer, inflammation, varicose veins, asymptomatic, hemorrhoids, hemorrhoids, internal without complication, hemorrhoids, internal without complication, hemorrhoids, external without complication, hemorrhoids, external thrombosed, hemorrhoids, varicose veins of other sites, esophageal varices without bleeding, esophageal varices without bleeding, varicocele, noninfective disorders of lymphatic channels, postmastectomy lymphedema syndrome, hypotension, orthostatic hypotension, iatrogenic hypotension, other disorders of circulatory system, other specified disorders of circulatory system, and unspecified venous insufficiency. Other examples of cardiac conditions include, without limitation, coronary artery occlusion (e.g., resulting from or associated with lipid/cholesterol deposition, macrophage/inflammatory cell recruitment, plaque rupture, thrombosis, platelet deposition, or neointimal proliferation); ischemic syndromes (e.g., resulting from or associated with myocardial infarction, stable angina, unstable angina, coronary artery restenosis or reperfusion injury); cardiomyopathy (e.g., resulting from or associated with an ischemic syndrome, a cardiotoxin, an infection, hypertension, a metabolic disease (such as uremia, beriberi, or glycogen storage disease), radiation, a neuromuscular disease, an infiltrative disease (such as sarcoidosis, hemochromatosis, amyloidosis, Fabry's disease, or Hurler's syndrome), trauma, or an idiopathic cause); arrhythmia or dysrrhythmia (e.g., resulting from or associated with an ischemic syndrome, a cardiotoxin, adriamycin, an infection, hypertension, a metabolic disease, radiation, a neuromuscular disease, an infiltrative disease, trauma, or an idiopathic cause); infection (e.g., caused by a pathogenic agent such as a bacterium, a virus, a fungus, or a parasite); and an inflammatory condition (e.g., associated with myocarditis, pericarditis, endocarditis, immune cardiac rejection, or an inflammatory conditions resulting from one of idiopathic, autoimmune, or a connective tissue disease).

### EXAMPLES

The following examples are provided for illustrative purposes only and are not intended to limit the scope of the invention.

### Example 1

### Analysis of DMB Levels in Dog Plasma

Sodium 2,2-dimethylbutyrate is administered to dogs as a part of a drug development program that includes toxicity studies. Dog plasma samples, collected in these studies, require bioanalytical analysis for concentration determination of sodium 2,2-dimethylbutyrate using a validated method. The quantitative data obtained is used to calculate the dog toxicokinetic parameters for toxicology studies with sodium 2,2-dimethylbutyrate. The objective of this study is to validate the LC-MS/MS method for the analysis of sodium 2,2-dimethylbutyrate in dog plasma. This study was conducted to validate the LC-MS/MS method for the analysis of sodium 2,2-dimethylbutyrate in K3 EDTA dog plasma.

Sodium 2,2-dimethylbutyrate and the added internal standard, DMV, were extracted from dog plasma using protein precipitation. The supernatant was dried, reconstituted, and derivatized to create benzyl amides of the analyte and internal standard. The resulting sample was dried and reconstituted for analysis by High Performance Liquid Chromatography (HPLC) on a reverse phase HPLC column. The analyte and internal standard were detected and quantitated by Tandem Mass Spectrometry. Calibration was accomplished by a 1/x2 weighted linear regression of the ratio of the peak areas of analyte to internal standard (sodium 2,2-dimethylbutyrate/DMV) to the corresponding nominal concentration sodium 2,2-dimethylbutyrate.

The following procedures describe requirements for the validation which were completed in this study: System Suitability, Specificity Linearity, Accuracy, Intra-day variation, Precision, Intra-day variation, Sensitivity, Lower Limit of Quantitation (LLOQ) Stability, Multiple freeze/thaw cycles in dog plasma Bench-top stability in dog plasma, Long term storage stability in dog plasma Recovery from dog plasma, and Dilution (10×).

Test Article

Sodium 2,2-Dimethylbutyrate

| | |
|---|---|
| Received from: | Frontage Laboratories, Inc. |
| Formula: | C6H11NaO2 |
| Molecular weight: | 138.14 |
| Lot Number: | DZ122702 |
| Storage: | −20° C. freezer in desiccator |
| Purity: | 98.8% |
| Expiration: | none provided |
| Retest Date: | December 2008 |
| Characterization: | GMP |

Internal Standard

2,2-Dimethylvaleric acid (DMV)

| | |
|---|---|
| Received from: | Sigma-Aldrich |
| Formula: | CH3CH2CH2C(CH3)2COOH |
| Molecular weight: | 130.18 |
| Lot Number: | 372790 |
| Storage: | Refrigerate |
| Purity: | 98.5% |
| Density | 0.918 |
| Expiration: | none provided |
| Retest Date: | none provided |
| Characterization: | none |

Reagents and Matrix

The reagents below are used in the study. All reagent lot numbers are documented in the raw data.

| Reagent | Description |
|---|---|
| Water | HPLC Grade, Fisher |
| Acetonitrile | HPLC Grade, Fisher |
| Formic acid | ACS Certified 96%, Aldrich |
| Sodium hydroxide | ACS Certified, Fisher |
| Benzylamine | Plus >99.5%, Fluka |
| N,N-diisopropylethylamine | Redistilled 99.5%, Sigma-Aldrich |
| Dexo-Fluor | 50% in THF, Fluka |
| K3 EDTA dog plasma from | Lot numbers: BGLBREC 24612, |
| Bioreclamation, Inc., | 25451, 25452, 25453, 25455, |
| 290 Duffy Avenue, | 25456, 27343 |
| Hicksville, New York | |
| 11801 | |

Equipment

The following equipment was used in the study:

| Equipment Item | Description |
|---|---|
| Mass Spectrometer | PE Sciex API 3000 |
| HPLC Autosampler | CTC Analytics LC PAL, Shimadzu SIL-10ADvp |
| HPLC Pumps | Shimadzu LC-10ATvp |
| HPLC Controller | Shimadzu SCL-10Avp |
| HPLC Column | Phenomenex Synergi 4μ, RP MAX, 150 × 2 mm |
| Balance | Sartorius ME 21 5P |
| Centrifuge | Eppendorf Model 5417R |
| Centrifuge | Jouan Model C-412 |
| Vortexer | VWR Scientific Multi-Tube Vortexer |
| Vortexer | Fisher Vortex Genie 2 |
| Evaporator | Zymark Turbo Vap ® LV |
| Pipetters | Rainin, Gilson variable volume |
| Centrifuge tubes | Fisher, 15 mL glass |
| Centrifuge tubes | Fisher, 2 mL plastic |
| Glassware | Miscellaneous volumetric flasks, |
| | graduate cylinders, etc. |

Dilutions were generally made as described below, however, weights and volumes of stock solutions may have varied. These changes are documented in the raw data.

Miscellaneous Solutions and Mobile Phase

Mobile Phase A: 0.5% (v/v) formic acid in water: An aliquot of 5.0 mL formic acid was added to 1 liter of degassed HPLC water and mixed. The solution was stored under ambient conditions, and assigned an expiration date of three months. Mobile Phase B: 0.5% (v/v) formic acid in acetonitrile: An aliquot of 5.0 mL formic acid was added to 1.0 liter of HPLC acetonitrile and mixed. The solution was stored under ambient conditions, and assigned an expiration date of three months. Dilution Solution: acetonitrile/water (50/50, v/v): An aliquot of 50 mL acetonitrile was combined and mixed with 50 mL water. The solution was stored under ambient conditions, and assigned an expiration date of three months. 1M Sodium hydroxide solution: An amount of 4.0 g sodium hydroxide was added to a 100 mL volumetric flask. The flask was diluted to the mark with water and mixed. The solution was stored under ambient conditions, and assigned an expiration date of three months. Reconstitution Solution: acetonitrile/water (25/75, v/v): An aliquot of 25 mL acetonitrile was combined and mixed with 75 mL water. The solution was stored under ambient conditions, and assigned an expiration date of three months. 5% Benzylamine in acetonitrile solution: An aliquot of 0.5 mL benzylamine was transferred to a 10 mL volumetric flask. The flask was diluted to the mark with acetonitrile and mixed. The solution was stored under ambient conditions, and assigned an expiration date of three months. 5% N,N-diisopropylethylamine in acetonitrile solution: An aliquot of 0.5 mL N,N-diisopropylethylamine was transferred to a 10 mL volumetric flask. The flask was diluted to the mark with acetonitrile and mixed. The solution was stored under ambient conditions, and assigned an expiration date of three months. 60 mg/mL Deoxo-Fluor in acetonitrile solution: An aliquot of 1.20 mL Deoxo-Fluor (50% in THF) was transferred to a 10 mL volumetric flask. The flask was diluted to the mark with acetonitrile and mix. The solution was stored at approximately 4° C. under argon, and assigned an expiration date of three months.

Sodium 2,2-Dimethylbutyrate Stock Solutions and Working Stock Solutions

The following series of Primary Stock Solutions and Working Stock Solutions were prepared. The procedures that follow are an example of standard solution preparation. The exact weights and volumes are recorded in the raw data. All solutions were stored in a refrigerator at approximately 4° C. The standard solutions were assigned an expiration period of 3.5 months.

1000 ug/mL sodium 2,2-dimethylbutyrate Primary Stock Solution

An amount of 25 mg of sodium 2,2-dimethylbutyrate (correct for purity and sodium salt content) was weighed and transferred to a 25 mL volumetric flask. The flask was diluted to the mark with Dilution Solution, and mixed well and sonicated to ensure dissolution. The sodium 2,2-dimethylbutyrate Working Stock Solutions were prepared according to the prototypical dilution scheme listed below. The standards were diluted in 10 mL volumetric flasks with Dilution Solution, and transferred to scintillation vials for storage.

| Sodium 2,2- | | |
|---|---|---|
| Dimethylbutyrate | Volume of | |
| Working Stock | 1,000 μg/mL Sodium | Final Volume of |
| Solution | 2,2-Dimethylbutyrate | Working Stock |
| Concentration | Primary Stock | Solution (mL) |
| 500 | 5.0 | 10.0 |
| 200 | 2.0 | 10.0 |
| 100 | 1.0 | 10.0 |
| 40 | 0.40 | 10.0 |
| 10 | 0.10 | 10.0 |
| 4.0 | 0.040 | 10.0 |
| 2.0 | 0.020 | 10.0 |

DMV Internal Standard Stock Solutions and Working Stock Solutions

The following series of Primary Stock Solutions and Working Stock Solutions were prepared. The procedures that follow are an example of standard solution preparation. The exact weights and volumes are recorded in the raw data. All solutions were stored in a refrigerator at approximately 4° C. The standard solutions were assigned an expiration period of two months.

1000 ug/mL DMV Primary Stock Solution: An amount of approximately 25 mg of DMV (correct for purity) was weighed and transferred to a 25 mL volumetric flask. The flask was diluted to the mark with Dilution Solution, and mixed well and sonicated to ensure dissolution.

10 ug/mL DMV Working Stock Solution: An aliquot of 0.10 mL of the 1,000 ug/mL DMV Primary Stock Solution was added to a 10 mL volumetric flask. The flask was diluted to the mark with Dilution Solution and mixed well.

System Suitability Solutions

A system suitability solution was prepared by fortifying the following standard solution aliquots into a 15 mL glass centrifuge tube, and processing through the analytical procedure: 10 μL of the 10 μg/mL sodium 2,2-dimethylbutyrate Working Stock Solution; and 10 μL of the 10 μg/mL DMV Working Stock Solution

Specificity Solutions.

Extracts of Control Plasma: Control dog plasma from six different lots were extracted according to the extraction procedure to evaluate the method specificity. Extracts of Control Plasma Fortified with Internal Standard: Control dog plasma from six different lots were fortified with internal standard and extracted according to the extraction procedure to evaluate the method specificity.

sodium 2,2-dimethylbutyrate Stock Solutions and Working Stock Solutions for Quality Control Samples

The following series of Primary Stock Solutions and Working Stock Solutions were prepared. The procedures that follow are an example of standard solution preparation. The exact weights and volumes are recorded in the raw data. All solutions were stored in a refrigerator at approximately 4° C. The sodium 2,2-dimethylbutyrate standard solutions were assigned an expiration period of 3.5 months.

1000 ug/mL sodium 2,2-dimethylbutyrate Primary Stock Solution: An amount of 25 mg of sodium 2,2-dimethylbutyrate (correct for purity and sodium salt content) was weighed and transferred to a 25 mL volumetric flask. The flask was diluted to the mark with Dilution Solution, and mixed well and sonicated to ensure dissolution. The sodium 2,2-dimethylbutyrate Working Stock Solutions were prepared according to the prototypical dilution scheme listed below. The standards were diluted in 10 mL volumetric flasks with Dilution Solution, and transferred to scintillation vials for storage.

| | | Volume of | Final Volume of |
|---|---|---|---|
| | Sodium 2,2- | 1,000 μg/mL Sodium | QC Working |
| | Dimethylbutyrate | 2,2-Dimethylbutyrate | StockS olution |
| QC-Level | QC Working | QC | (mL) |
| QC-High | 400 | 4.0 | 10.0 |
| QC-Mid | 100 | 1.0 | 10.0 |
| QC-Low | 6.0 | 0.060 | 10.0 |
| LLOQ | 2.0 | 0.020 | 10.0 |

Preparation of Dog Plasma Calibration Standards

The following aliquots of Working Solutions were added to 0.1 mL of dog plasma to prepare 7 calibration standards.

| | Sodium 2,2- | |
|---|---|---|
| Concentration of Sodium | Dimethylbutyrate | Fortified |
| 2,2-Dimethylbutyrate in | working stock | volume |
| plasma (ug/mL) | solution (ug/mL) | (iL) |
| Control (without Int. Std.) | none | none |
| Control + IS (with Int. Std.) | none | 10 |
| 0.20 | 2.0 | 0 |
| 0.40 | 4.0 | 10 |
| 1.0 | 10 | 10 |
| 4.0 | 40 | 10 |
| 10.0 | 100 | 10 |
| 20.0 | 200 | 10 |
| 50.0 | 500 | 10 |

Preparation of Dog Plasma Qc Samples

Three levels of Quality Control samples (QC-Low, QC-Mid and QC-High), at 0.60, 10 and 40 μg/mL sodium 2,2-dimethylbutyrate were prepared. Samples were also prepared at the Low Limit of Quantitation (LLOQ) at 0.20 μg/mL sodium 2,2-dimethylbutyrate. The following aliquots of sodium 2,2-dimethylbutyrate Working Stock Solutions were added to 0.1 mL of dog plasma, and processed as fresh QC samples, or used for storage stability experiments.

| | | Sodium 2,2- | |
|---|---|---|---|
| | Concentration of | Dimethylbutyrate | Fortified |
| QC Sample | Sodium 2,2- | QC | volume |
| LLOQ | 0.20 | 2.0 | 10 |
| QC-Low | 0.60 | 6.0 | 10 |
| QC-Middle | 10.0 | 100 | 10 |
| QC-High | 40.0 | 400 | 10 |

Preparation of Recovery Samples

Triplicate QC-Low and QC-High samples were generated substituting water instead of plasma. These samples were analyzed during one of the validation runs and compared to 5 replicates of QC-Low and QC-High plasma samples.

Preparation of 10-Fold Dilution QC Samples

A dilution QC sample (˜100 μg/mL sodium 2,2-dimethylbutyrate in dog plasma) was prepared by fortifying an aliquot of 0.9029 mL dog plasma with 0.0971 mL of the 1,029.79 ug/mL sodium 2,2-dimethylbutyrate QC Primary Stock Solution. Three-0.010 mL aliquots of the dilution QC sample were diluted with 0.090 mL control plasma to obtain a 10-fold dilution. These diluted plasma samples were fortified with internal standard and processed through the analytical procedure.

Sample Extraction Procedure

Control dog plasma was thawed at ambient temperature or in tepid water. As needed, the control plasma was centrifuged ˜3,500 rpm for 5 minutes. An aliquot of 0.10 mL of plasma was transferred into individual centrifuge tubes. The 0.10 mL of plasma was fortified with 10 ul of working stock solution for the calibration curve standards and QC samples, respectively. The tubes were briefly mixed. All plasma samples, except the plasma control, were fortified with 10 ul of the 10.0 ug/mL DMV Working Stock Solution and briefly mixed. The control+IS sample and Dilution QCs were fortified with 10 ul Dilution Solution and briefly mixed. The control sample was fortified with 20 ul of the Dilution Solution and briefly mixed. An aliquot of 1.0 mL of acetonitrile was added to the tube followed by vortexing for 2 minutes. The tube was centrifuged at ˜14,000 rpm for 10 minutes. The supernatant was transferred to a 15 mL glass centrifuge tube. An aliquot of 10 μL of the 1.0 M sodium hydroxide solution was added to the tube. The supernatant was dried in the TurboVap at approximately 37° C. under a nitrogen flow. The residue was dissolved in 0.5 mL acetonitrile by vortexing for 5 seconds. Aliquots of 10 μL for both the 5% benzylamine solution and the 5% N,Ndiisopropylethylamine solution were added to the tube, and vortexed for 5 seconds. The sample was stored at −20° C. for approximately 20 minutes. An aliquot of 10 μL of the 60 mg/mL cooled Deoxo-Fluor solution was added to the tube and vortexed 5 seconds. The sample was stored at −20° C. for approximately 20 minutes. The extract was dried in the TurboVap at approximately 37° C. under a nitrogen flow. The dried extract was reconstituted in 1.0 mL of the Reconstitution Solution and vortexed 10 seconds. The tube was centrifuged ˜3,500 rpm for 5 minutes. The extract was transferred to an autosampler vial or 96-well plate for LCMS/MS analysis.

The following LC-MS/MS conditions were applied for the analysis of sodium 2,2-dimethylbutyrate in dog plasma:

HPLC Parameters:

| | |
|---|---|
| Column: | Phenomenex Synergi RP Max 4 i, 150 × 2 mm, |
| | with a guard column cartridge or prefilter |
| Column flow rate: | 0.3 mL/min. |
| | The flow was increased to 0.4 mL/min after |
| | peak elution to ensure matrix removal from |
| | the column. The flow was diverted to waste |
| | before and after peak elution for some runs. |
| Column temperature: | Ambient |
| Injection volumes | 2 or 5 μL |
| used in the study: | |
| Mobile Phase A: | 0.5% formic acid in water |
| Mobile Phase B: | 0.5% formic acid in acetonitrile |
| Mode: | Isocratic, 80% Mobile Phase B |
| Run time: | 5 minutes |

Mass Spectrometry Parameters:

| Mass Spectrometer: | Applied Biosystems API 3000 | | |
|---|---|---|---|
| Ionization Interface: | TurboIon Spray (electrospray) | | |
| Ionization mode: | Positive | | |
| Transition Ion | | Precursor Ion | |
| Parameters: | Compound | Q1 Mass (amu) | Q3 Mass (amu) |
| | Sodium 2,2- | 206 | 71 |
| | DMV | 220 | 85 |

Calculations

The peak areas of sodium 2,2-dimethylbutyrate, and the internal standard DMV, were integrated by using the Analyst (Version 1.1) software provided by PE Sciex. The calibration curves were generated via least-square linear regression analysis. The general equation is as follows:

y=a+b*x

where, y=Peak area ratio (analyte area to internal standard area); x=Analyte calibration standard concentration, nominal; a=Intercept; b=Slope. All reported concentration data were calculated from 1/x² weighted linear regression curves.

The samples were analyzed in one day to determine precision, accuracy, linearity. System suitability solutions were analyzed prior to each sample set. One set of calibration curve mixed standards at the concentrations of 0.2, 0.4, 1.0, 4.0, 10, 20, and 50 μg/mL sodium 2,2-dimethylbutyrate in dog plasma. LLOQ samples in five replicates at 0.2 μg/mL sodium 2,2-dimethylbutyrate in dog plasma. QC-Low samples in five replicates at 0.6 μg/mL sodium 2,2-dimethylbutyrate in dog plasma. QC-Mid samples in five replicates at 10 μg/mL sodium 2,2-dimethylbutyrate in dog plasma. QC-High samples in five replicates at 40 μg/mL sodium 2,2-dimethylbutyrate in dog plasma. One dog plasma control sample (blank) and one dog plasma control sample fortified with internal standard (zero). System suitability samples (n=6) containing sodium 2,2-dimethylbutyrate and DMV.

For the remaining validation tests, a calibration curve and triplicates QCs at the low, mid and high levels were analyzed with each sample set. The following samples were also analyzed either in conjunction with one of the precision and accuracy runs or in one of the additional validation runs: Samples from six lots of control dog plasma for specificity. Two concentration levels of unextracted QC-samples in triplicate (solvent standards) were analyzed for the evaluation of the recovery of sodium 2,2-dimethylbutyrate and DMV in dog plasma. Two levels of QC samples (Low QC and High QC in triplicate) were subjected to three freeze/thaw cycles at approximately −70° C. prior to extraction to evaluate freeze/thaw stability. Two levels of QC samples (Low QC and High QC in triplicate) were placed on the bench top for approximately 17 hours prior to extraction for the evaluation of the bench top stability. Triplicate low QC samples and triplicate high QC samples were stored for one month and three months in a freezer at approximately −70° C., and then extracted and analyzed to evaluate long term stability in dog plasma. Three aliquots of a dilution QC sample diluted 10-fold.

Statistical calculations in the report tables were calculated from unrounded concentration values taken directly from the raw data. The concentration values were rounded for display purposes.

The system suitability was evaluated each day that dog plasma validation samples were analyzed. One system suitability solution was injected six times. The precision for all system suitability analyses is shown in Table 1. The intra-day coefficient of variation percent (CV %) did not exceed 10.5% for sodium 2,2-dimethylbutyrate, and 10.8% for DMV. The LC-MS/MS method was found to be suitable for the validation.

The following samples were prepared and analyzed to evaluate specificity of the method. Chromatograms of these samples were evaluated for the presence of any interference peak at the retention time regions of sodium 2,2-dimethylbutyrate and DMV. Extracts of dog control plasma from six different lots and extracts of dog control plasma from six different lots fortified with internal standard.

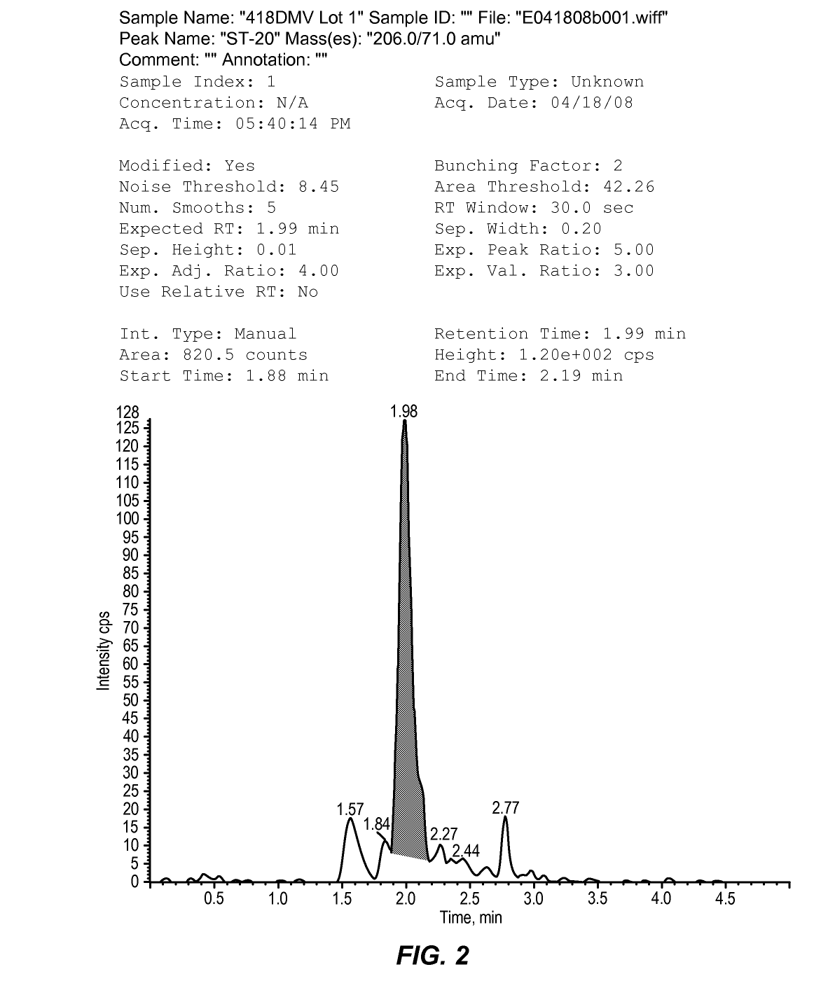

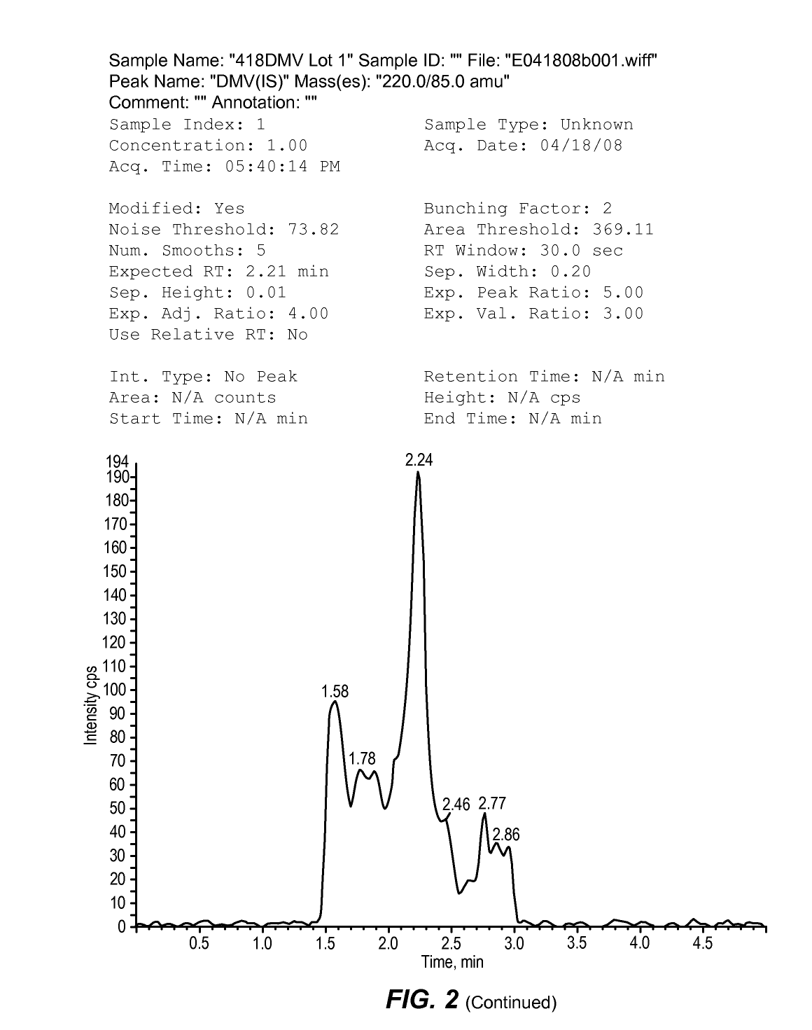

The specificity samples contained apparent sodium 2,2-dimethylbutyrate at a concentrations ranging from 13% to 28% of the LLOQ. The sodium 2,2-dimethylbutyrate peak in the specificity samples was not due to injector carryover. Similar, apparent levels of Sodium 2,2-Dimethylbutyrate in control plasma were observed during the full method validation. It was determined from experiments during the full method validation that the sodium 2,2-dimethylbutyrate levels found in control plasma are not related to the plasma, but can be considered background levels inherent in the method. Though the sodium 2,2-dimethylbutyrate background can vary, it is at a low level where quantitation is not affected. FIG. 2 is a representative chromatogram of a plasma control, which shows the sodium 2,2-dimethylbutyrate background levels.

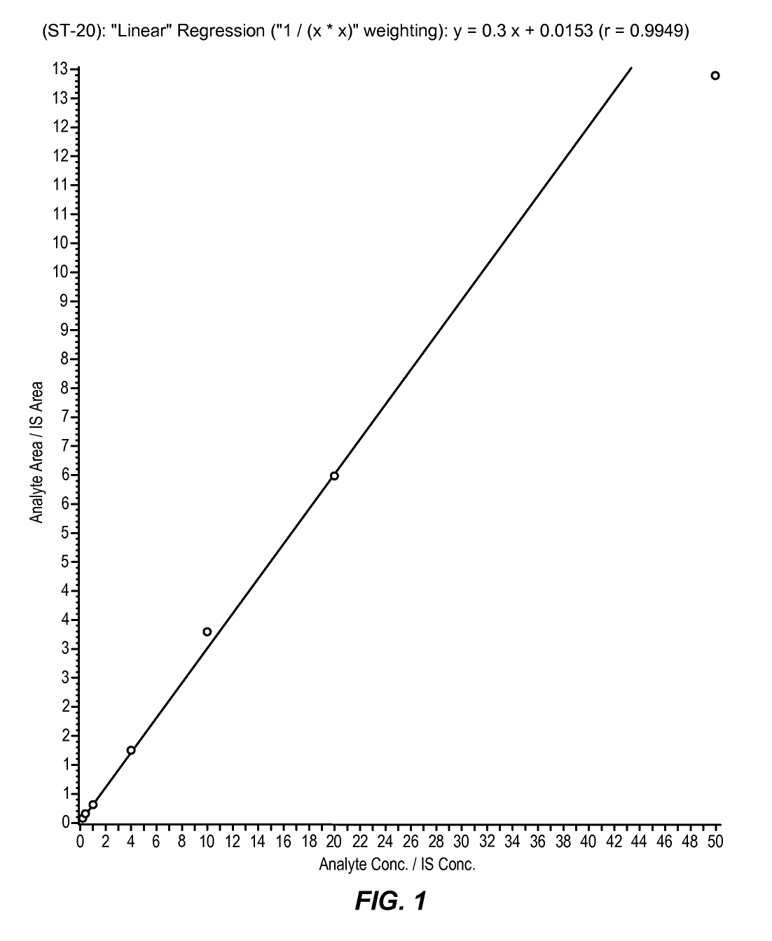

The relationship between the concentration of the analyte and the peak area ratios of the compound to internal standard was established. The parameters of the calibration curves for sodium 2,2-dimethylbutyrate are listed in Table 7.2. A typical calibration curve, depicted in FIG. 1, shows linearity for sodium 2,2-dimethylbutyrate over the concentration range of 0.20 μg/mL to 50 μg/mL. Correlation coefficients were >0.9949, satisfying the acceptance criteria of r≧0.990.

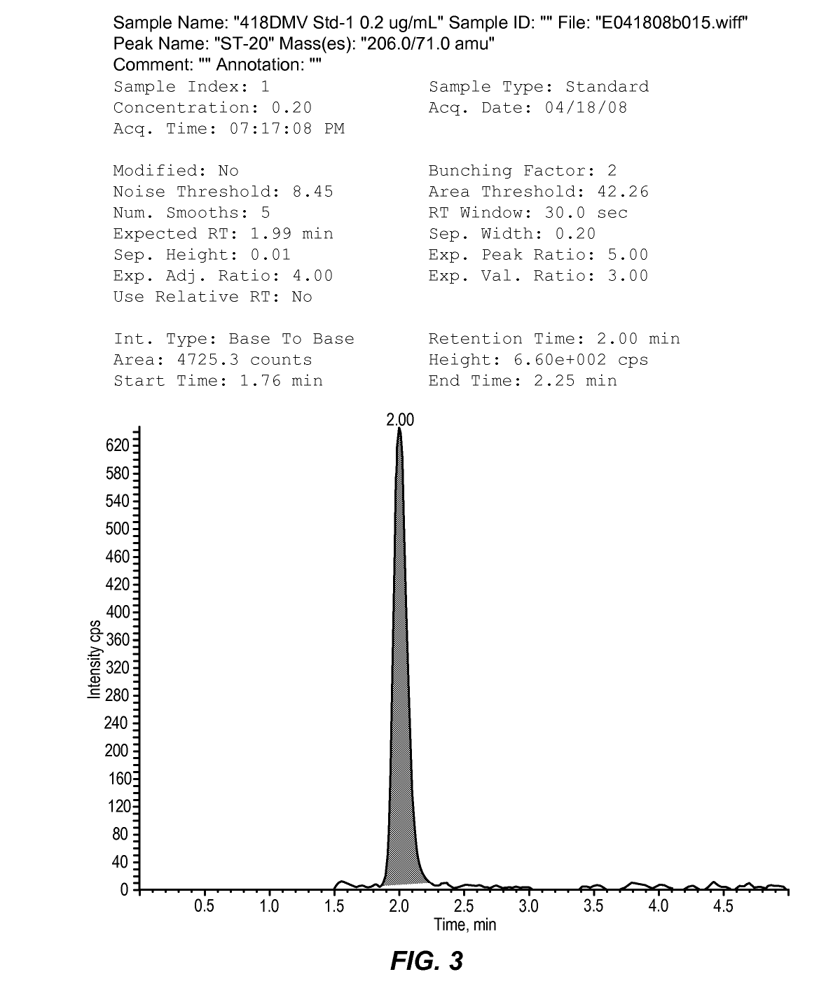

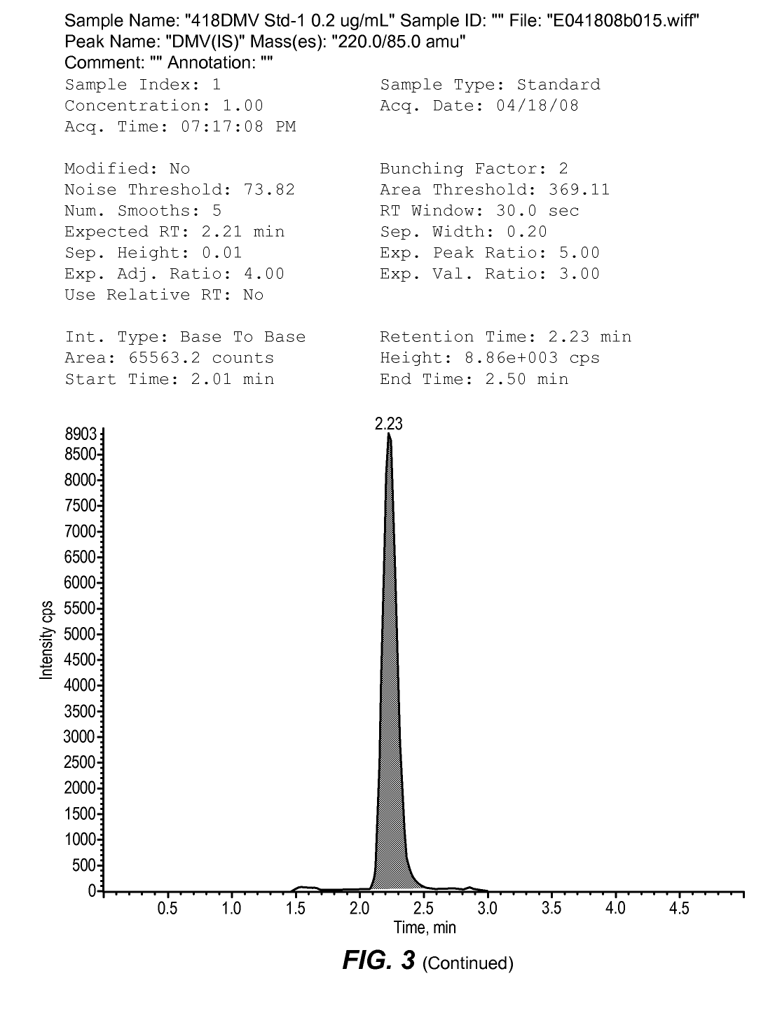

Back-calculated concentrations of QC samples (LLOQ, QC-Low, QC-Mid, and QC-High) for sodium 2,2-dimethylbutyrate were used for the statistical treatment of intra-day accuracy and precision. The data are shown in Table 3. Overall precision of the method was measured by the percent coefficient of variation (CV %). Table 3 shows the CV % for the LLOQ QC was 5.3%. The CV % range for the Low-, Mid-, and High-QCs was from 1.9% to 4.9%. These values are within the CV % acceptance limits of <20% for LLOQQCs and <15% for Low-, Mid-, and High-QCs. Overall accuracy of the method was measured by the percent relative error (RE %), which was determined by comparing the mean values of the measured concentrations with the nominal concentrations of the analyte. Table 3 shows the RE % for the LLOQ QC was −10.7%. The RE % range for the Low-, Mid-, and High-QCs was from −4.9% to 3.7%. Thus, all RE % values meet acceptance criteria (+20% for LLOQ-QCs and +15% for Low-, Mid-, and High-QCs). The data indicate that the method provides good intra-day precision and accuracy over the LLOQ to QC-High range for sodium 2,2-dimethylbutyrate. Typical chromatograms of sodium 2,2-dimethylbutyrate in plasma samples are presented in FIGS. 3-7.

Sensitivity (LLOQ)

The data for the LLOQ are presented in Table 3. The values of the CV % and RE % are 5.3% and −10.7%, respectively. All values are well within the acceptable limits of ≦20% for CV and ±20% RE, indicating that the lower limit of quantitation for this method is 0.2 μg/mL for sodium 2,2-dimethylbutyrate.

The recovery was evaluated for sodium 2,2-dimethylbutyrate and DMV. This was determined by comparison of the peak areas of plasma QC samples at Low-QC and High-QC levels versus those of fortified water blank samples (water substituted for plasma) at the same concentration levels. The data are listed in Table 4. In many cases, the CV % was >15% for the five replicates of plasma QCs or the three replicates of fortified water blanks. It is believed that the derivatization step in the procedure is the cause of this peak area variability. Therefore, the recovery obtained is an approximation. The recovery of sodium 2,2-dimethylbutyrate from dog plasma ranged from 61.8% to 72.2%. The recovery of DMV from dog plasma ranged from 65.3% to 75.7%.

The stability of sodium 2,2-dimethylbutyrate in dog plasma was evaluated at approximately −70° C. for three cycles using QC-Low and QC-High samples in triplicate. The freeze time was at least 12 hours, with a minimum thaw time of one hour. The results are shown in Table 5. The CV % values for the QC-Low and QC-High stability samples are 1.4% and 2.8%, respectively. To calculate the RE %, the measured mean concentration was compared to the nominal concentration. The RE % values for the QC-Low and QC-High stability samples are 1.9% and −8.1%, respectively. All CV % and RE % values fall within the limits of ≦15% and ±15%, respectively, indicating that sodium 2,2-dimethylbutyrate is stable in dog plasma after three freeze/thaw cycles.

Bench top stability was evaluated at room temperature for approximately 17 hours. Triplicate QC-Low and QC-High samples were extracted and analyzed after these storage conditions. The results are shown in Table 6. The CV % values for the QC-Low and QC-High stability samples are 3.2% and 3.4%, respectively. To calculate the RE %, the measured mean concentration was compared to the nominal concentration. The RE % values for the QC-Low and QC-High stability samples are 2.1% and −10.7%, respectively. All CV % and RE % values fall within the limits of ≦15% and ±15%, respectively, indicating that sodium 2,2-dimethylbutyrate is stable in dog plasma after ambient bench top storage for approximately 17 hours.

QC-Low and QC-High plasma samples, which had been stored in a freezer at approximately −70° C. for one month and three months (99 days), were extracted in triplicate and analyzed. The data is presented in Table 7. All CV % values and RE % values are within the acceptance criteria (≦15% and ±15% respectively). Therefore, sodium 2,2-dimethylbutyrate can be considered stable in dog plasma at approximately −70° C. for at least 99 days.

A quality control sample was prepared at a concentration of 100 μg/mL Sodium 2,2-Dimethylbutyrate in dog plasma. The QC sample was diluted 10-fold in three replicates with control plasma to obtain a concentration of sodium 2,2-dimethylbutyrate within the calibration range. The data from this analysis are presented in Table 8.

The CV % and RE % values for the dilution QC experiment were 0.9% and −1.5%, respectively. The data, which fall within acceptance limits for CV % (≦15%) and RE % (±15%), indicate that using a 10-fold dilution yields analytical results that are precise and accurate.

According to the COA for sodium 2,2-dimethylbutyrate, the standard was to be stored under ambient conditions in a desiccator. The protocol incorrectly listed storage under refrigerated conditions in a desiccator. During the study, the sodium 2,2-dimethylbutyrate neat standard was stored under frozen conditions (−20° C.) in a desiccator. This deviation had no impact on the study. Several weighings of sodium 2,2-dimethylbutyrate were previously made for stock solution stability analyses. The sodium 2,2-dimethylbutyrate stock solutions were found to be stable for 3.5 months, therefore, the neat standard must also be stable under frozen storage conditions.

For the specificity experiment, sodium 2,2-dimethylbutyrate was present in some controls at levels greater than 20% of the LLOQ level. This was a protocol deviation which specified that levels of sodium 2,2-dimethylbutyrate in plasma controls should be less than 20% of the LLOQ. This deviation had little effect on the study since the sodium 2,2-dimethylbutyrate background levels were at a low enough level that it did not interfere with the calibration curve and QCs.

The method presented here for the determination of sodium 2,2-dimethylbutyrate in dog plasma shows acceptable linearity, precision and accuracy for the calibration range of 0.2 μg/mL to 50 μg/mL. The method is specific for the internal standard, DMV, but did not meet the specificity criteria for Sodium 2,2-Dimethylbutyrate, since sodium 2,2-dimethylbutyrate was detected in blank plasma at a level up to 28% of the LLOQ concentration. It is believed that the sodium 2,2-dimethylbutyrate levels found in blank plasma are not related to the plasma, but can be considered background levels inherent in the method. The dog plasma can be diluted 10-fold and analyzed with acceptable precision and accuracy. At concentration levels within the calibration range, sodium 2,2-dimethylbutyrate in dog plasma is stable at room temperature on the bench top for at least 17 hours, and for three freeze/thaw cycles at approximately −70° C. Sodium 2,2-Dimethylbutyrate is stable in dog plasma for at least 99 days when stored at approximately −70° C. The recovery in dog plasma ranged from 61.8% to 72.2% for Sodium 2,2-Dimethylbutyrate, and 65.3% to 75.7% for DMV. These are approximate recovery ranges since the CV % values were high due to the variability introduced by the derivatization step.

TABLE 1
System Suitability

| | Sodium 2,2- | |
|---|---|---|
| Extraction | Dimethylbutyrate | DMV |
| Date | Peak Area | Peak Area |
| 1 Mo | 23306.5 | 78469.3 |
| | 24527.9 | 77762.7 |
| | 26136.1 | 77120.5 |
| | 24343.8 | 77308.8 |
| | 26094.6 | 77634.6 |
| | 24570.6 | 76353.4 |
| Mean | 24829.9 | 77441.6 |
| SD | 1097.4 | 707.3 |
| CV % | 4.4 | 0.9 |
| 2 Mo | 8092.2 | 33024.4 |
| | 7693.9 | 32627.3 |
| | 6679.5 | 28932.7 |
| | 6326.1 | 26618.9 |
| | 6453.9 | 26311.3 |
| | 6634.5 | 26354.6 |
| Mean | 6980.0 | 28978.2 |
| SD | 729.5 | 3138.5 |
| CV % | 10.5 | 10.8 |
| 3 Mo | 12124.4 | 39116.7 |
| | 11695.9 | 37940.0 |
| | 11738.8 | 39760.6 |
| | 12455.5 | 37981.8 |
| | 11178.2 | 38984.5 |
| | 11887.8 | 38201.4 |
| Mean | 11846.8 | 38664.2 |
| SD | 431.4 | 736.7 |
| CV % | 3.6 | 1.9 |

TABLE 2
Standard Curve Parameters for Sodium
2,2-Dimethylbutyrate Weighted (1/x2)

| Extraction | | | | Correlation |
|---|---|---|---|---|
| Date | n | Intercept | Slope | Coefficient |
| 1 Mo. | 7 | 0.0153 | 0.3 | 0.9949 |
| 2 Mo. | 7 | 0.0251 | 0.194 | 0.9961 |
| 3 Mo. | 7 | 0.0322 | 0.194 | 0.9969 |

TABLE 3
Accuracy and Precision of Sodium 2,2-Dimethylbutyrate
in QC Samples for Dog Plasma

| | LLOQ | QC-Low | QC-Mid | QC-High |
|---|---|---|---|---|
| Concentration (μg/mL) | 0.20 | 0.60 | 10.0 | 40.0 |
| | 0.186 | 0.627 | 10.4 | 38.3 |
| | 0.175 | 0.646 | 10.4 | 38.2 |
| | 0.166 | 0.639 | 10.3 | 36.8 |
| | 0.176 | 0.617 | 9.45 | 38.4 |
| | 0.190 | 0.581 | 9.51 | 38.6 |
| Mean | 0.179 | 0.622 | 10.0 | 38.0 |
| SD | 0.010 | 0.025 | 0.5 | 0.7 |
| CV % | 5.3 | 4.1 | 4.9 | 1.9 |
| RE % | −10.7 | 3.7 | 0.1 | −4.9 |

TABLE 4
Recovery of Sodium 2,2-Dimethylbutyrate and
DMV from Dog Plasma at the Two QC Levels

| | Peak Area | | | |
|---|---|---|---|---|
| | QC-Low | | QC-High | |
| | Sodium 2,2- | DMV | Sodium 2,2- | DMV |
| Plasma QC | 15268.2 | 75088.2 | 669758.9 | 58162.8 |
| | 17534.6 | 83820.1 | 730179.1 | 63720.3 |
| | 18467.6 | 89166.8 | 602941.4 | 54562.1 |
| | 10760.7 | 53696.4 | 857111.8 | 74396.7 |
| | 11464.1 | 60492.0 | 825052.3 | 71222.1 |
| Mean | 14699.0 | 72452.7 | 737008.7 | 64412.8 |
| SD | 3483.6 | 15086.3 | 105735.1 | 8405.7 |
| CV % | 23.7 | 20.8 | 14.3 | 13.0 |
| Fortified Water | 23705.8 | 108137.9 | 1249262.7 | 106305.7 |
| Blank | 24964.8 | 114512.9 | 690368.1 | 56590.0 |
| | 22634.1 | 110341.3 | 1124647.0 | 92352.3 |
| Mean | 23768.2 | 110997.4 | 1021425.9 | 85082.7 |
| SD | 1166.6 | 3237.7 | 293396.9 | 25642.7 |
| CV % | 4.9 | 2.9 | 28.7 | 30.1 |
| Recovery % | 61.8 | 65.3 | 72.2 | 75.7 |

TABLE 5
Freeze/Thaw Cycle Stability at approximately −70°
C. for Sodium 2,2-Dimethylbutyrate in Dog Plasma

Sodium 2,2-Dimethylbutyrate

| | QC-Low | QC-High |
|---|---|---|
| Concentration (μg/mL) | 0.60 | 40.0 |
| | 0.620 | 36.2 |
| | 0.612 | 37.9 |
| | 0.603 | 36.2 |
| Mean | 0.612 | 36.8 |
| SD | 0.009 | 1.0 |
| CV % | 1.4 | 2.8 |
| RE % | 1.9 | −8.1 |

TABLE 6
Bench Top Stability for Sodium 2,2-
Dimethylbutyrate in Dog Plasma

Sodium 2,2-Dimethylbutyrate

| | QC-Low | QC-High |
|---|---|---|
| Concentration (μg/mL) | 0.60 | 40.0 |
| | 0.605 | 36.7 |
| | 0.598 | 36.1 |
| | 0.635 | 34.4 |
| Mean | 0.613 | 35.7 |
| SD | 0.020 | 1.2 |
| CV % | 3.2 | 3.4 |
| RE % | 2.1 | −10.7 |

TABLE 7
Long-Term Freezer Stability at approximately −70°
C. for Sodium 2,2-Dimethylbutyrate in Dog Plasma

Sodium 2,2-Dimethylbutyrate

| | | |
|---|---|---|
| | QC-Low | QC-High |
| Concentration (μg/mL) | 0.60 | 40.0 |

1 Month Interval

| | | |
|---|---|---|
| | 0.572 | 35.5 |
| | 0.586 | 36.4 |
| | 0.567 | 36.3 |
| Mean | 0.575 | 36.1 |
| SD | 0.010 | 0.5 |
| CV % | 1.7 | 1.3 |
| RE % | −4.2 | −9.9 |

3 Month Interval

| | | |
|---|---|---|
| | 0.629 | 36.1 |
| | 0.621 | 37.1 |
| | 0.568 | 31.7 |
| Mean | 0.61 | 35.0 |
| SD | 0.03 | 2.9 |
| CV % | 5.5 | 8.3 |
| RE % | 1.0 | −12.5 |

TABLE 8
10-Fold Dilution QC Samples for Sodium
2,2-Dimethylbutyrate in Dog Plasma

| | Sodium 2,2- |
|---|---|
| Concentration (μg/mL) | 100 |
| | 98.3 |
| | 97.7 |
| | 99.5 |
| Mean | 98.5 |
| SD | 0.9 |
| CV % | 0.9 |
| RE % | −1.5 |

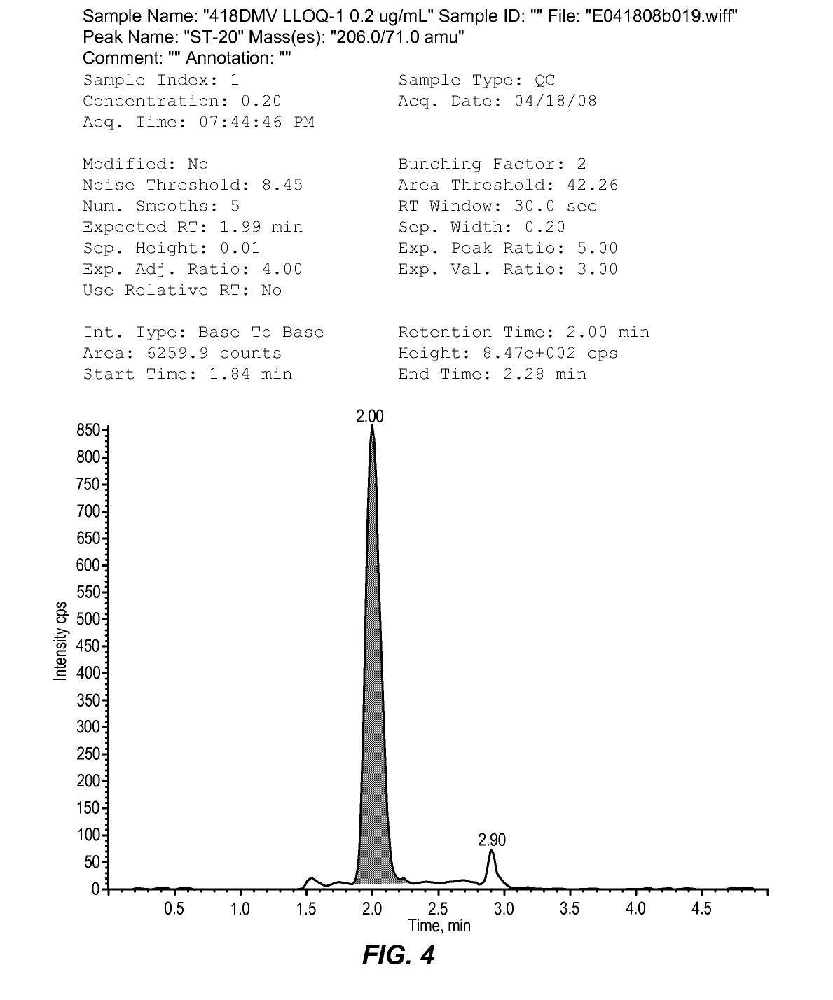

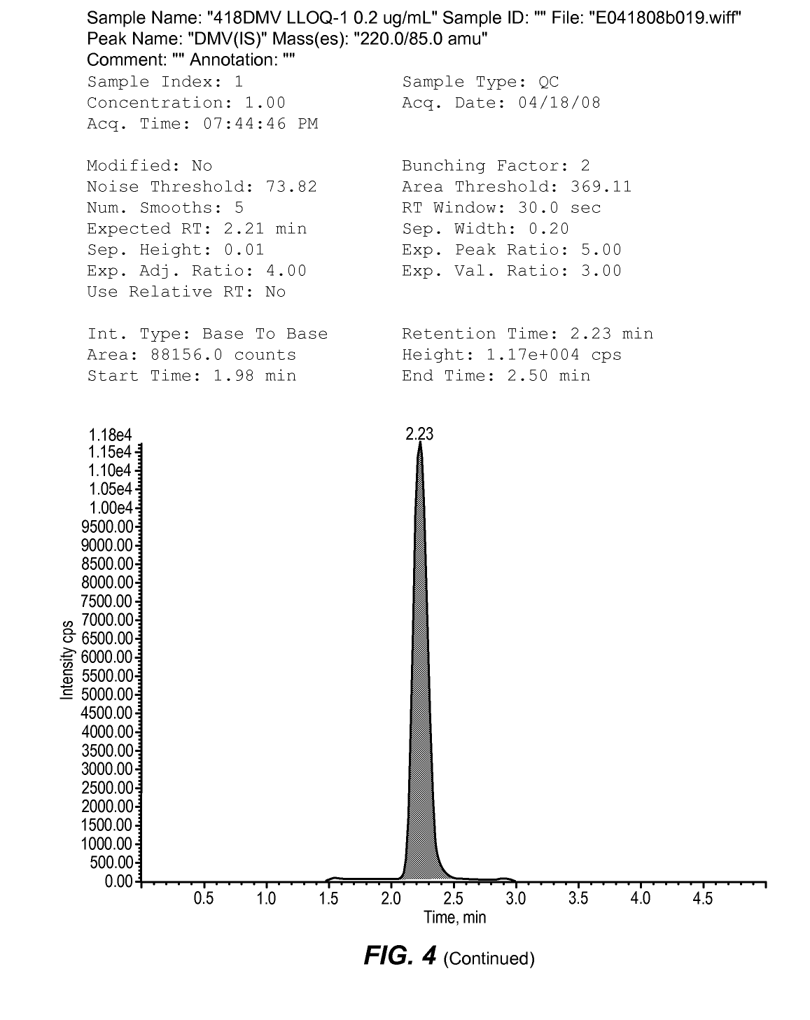

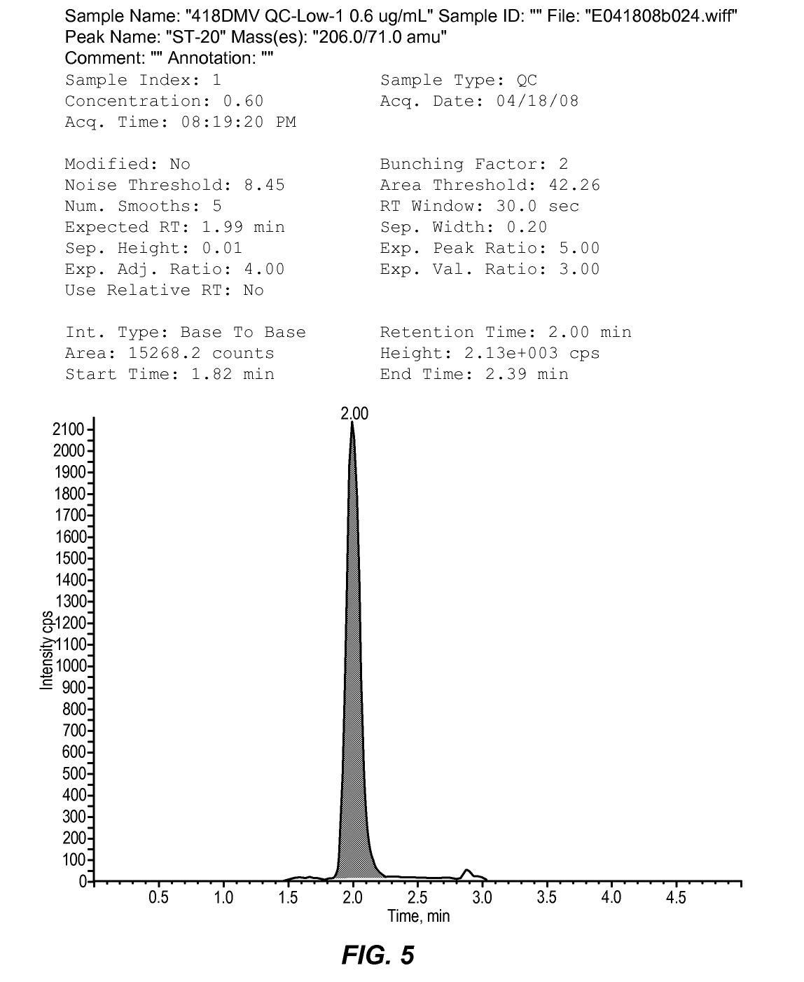

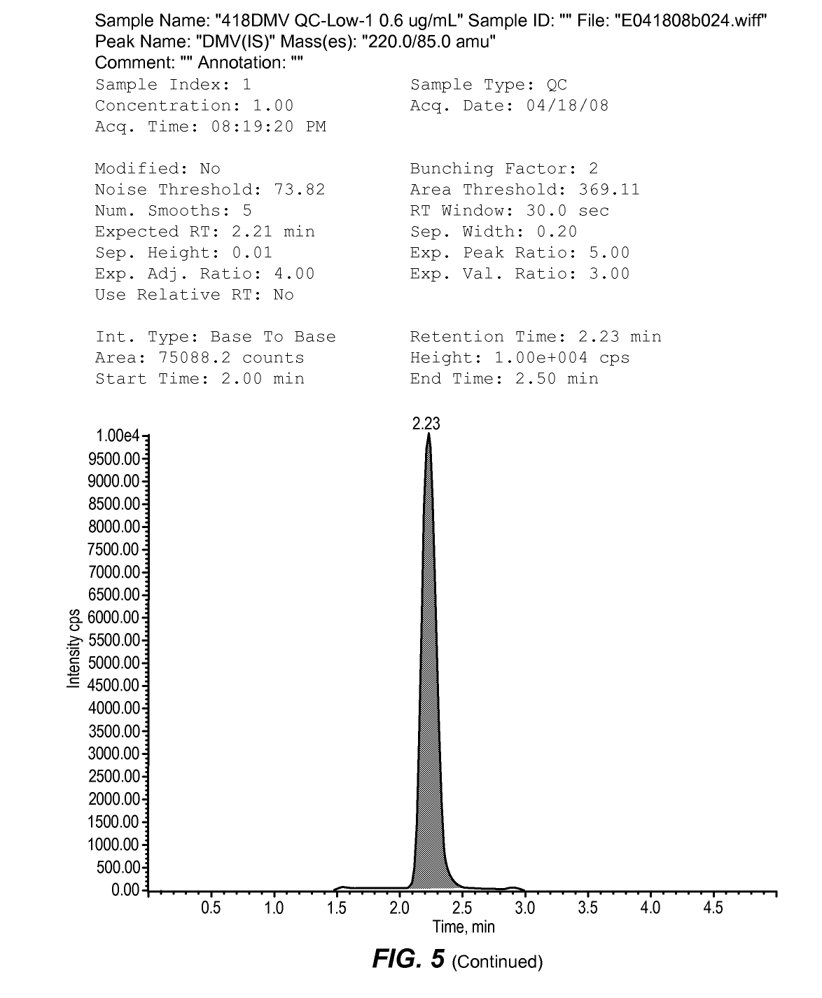

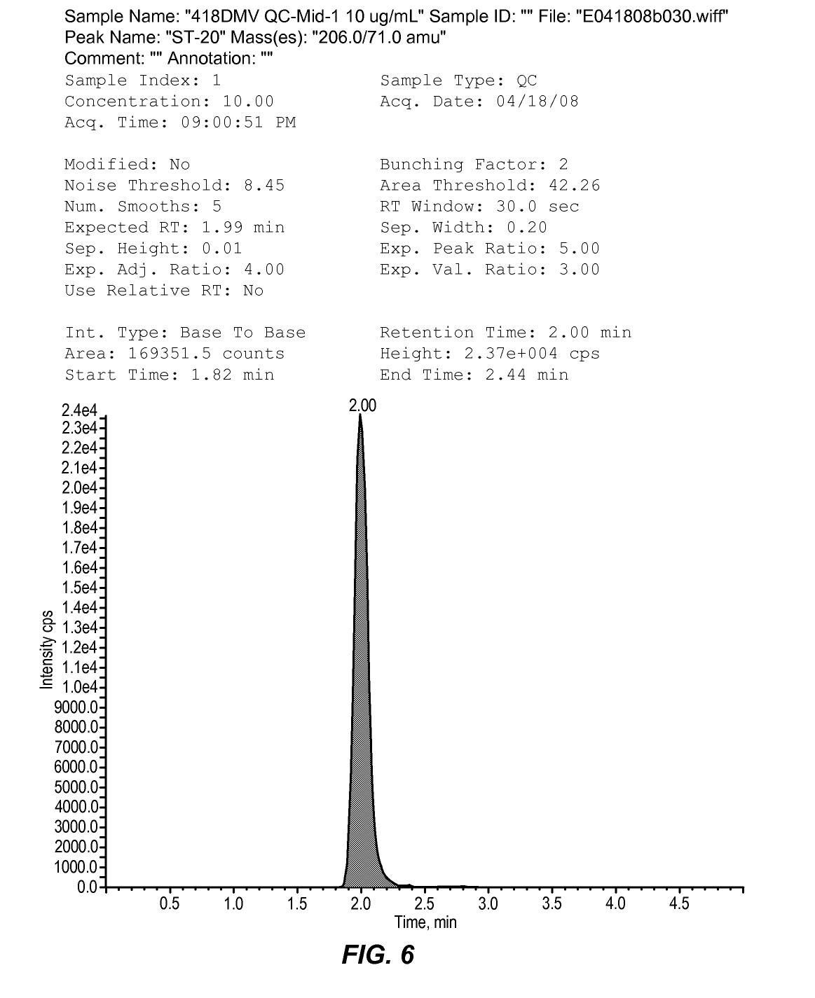

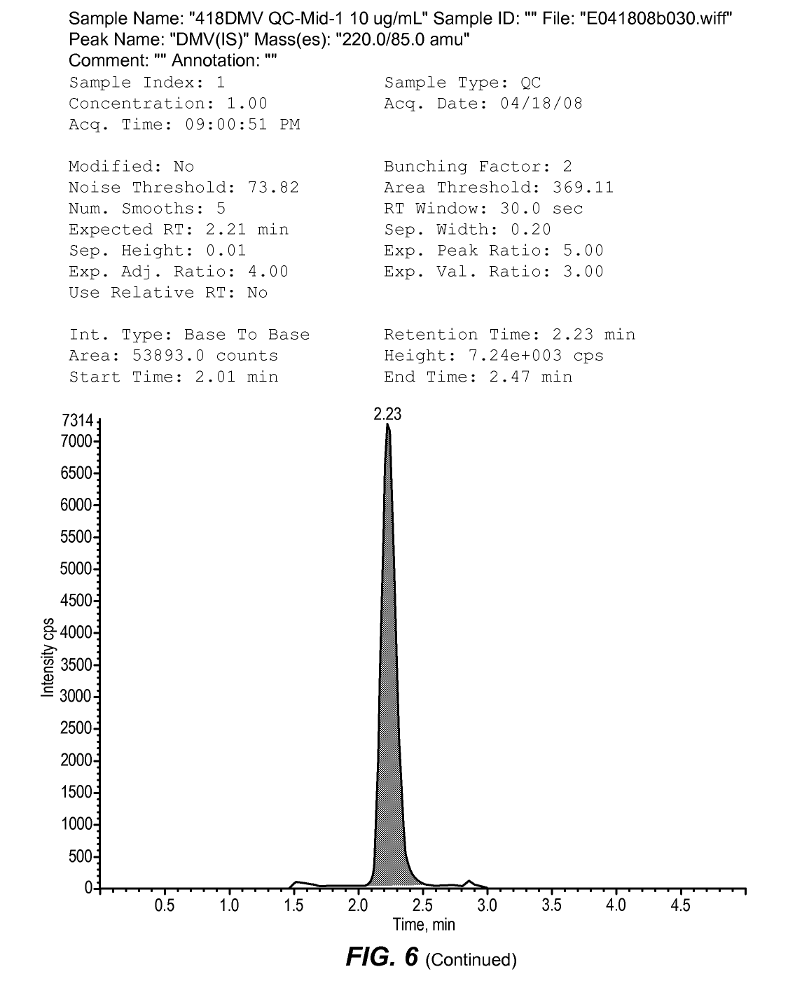

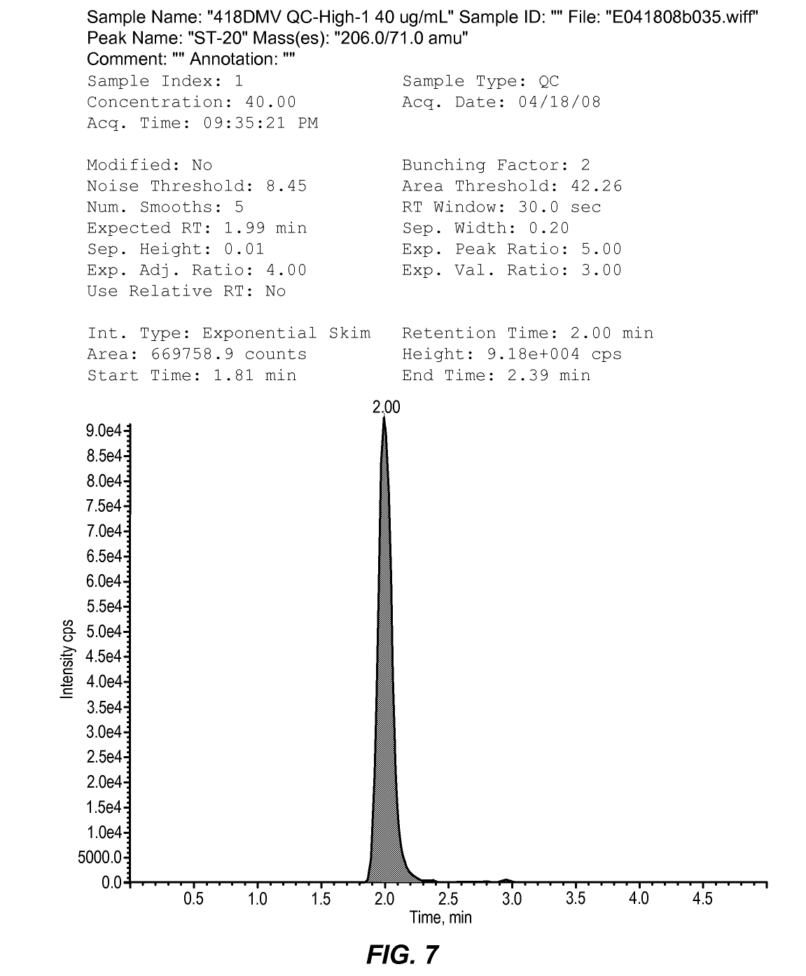

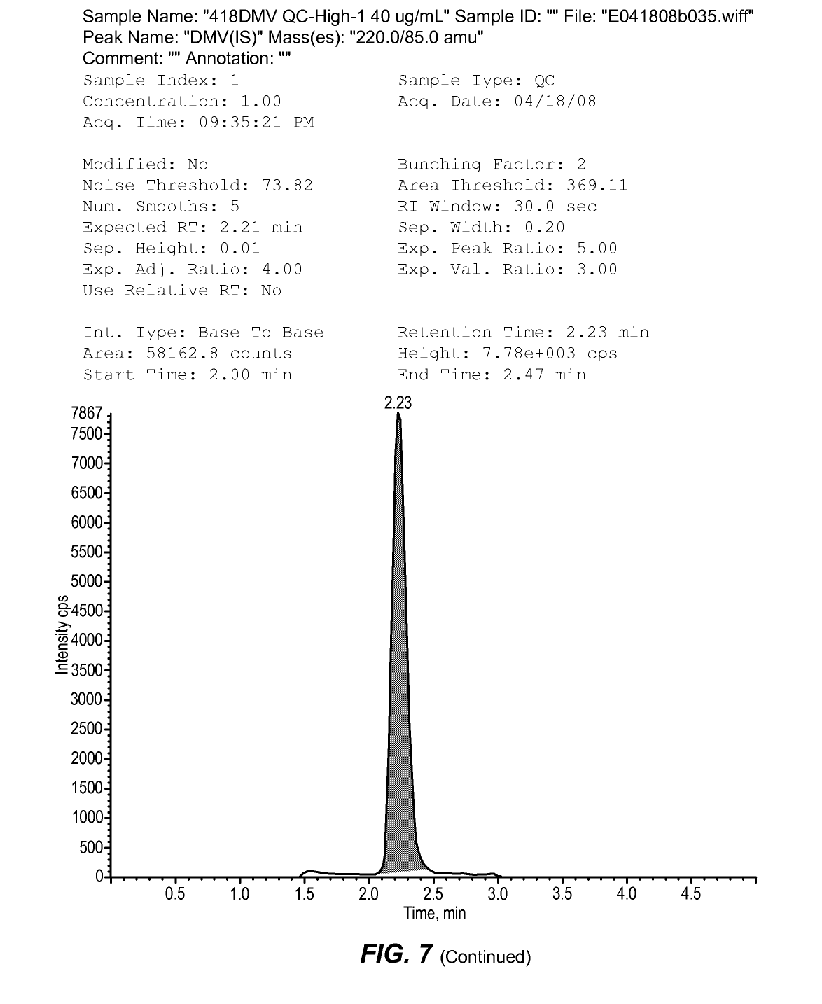

Representative chromatograms from the study are illustrated in FIGS. 1-7. FIG. 1 illustrates representative Calibration Curve. FIG. 2 illustrates representative Chromatograms of Control Dog Plasma sodium 2,2-dimethylbutyrate (top), DMV (bottom). FIG. 3 illustrates representative Chromatograms of Standard-1 (0.2 μg/mL sodium 2,2-dimethylbutyrate) in Dog Plasma Sodium 2,2-Dimethylbutyrate (top), DMV (bottom). FIG. 4 illustrates representative Chromatograms of LLOQ (0.2 ug/mL sodium 2,2-dimethylbutyrate) in Dog Plasma sodium 2,2-dimethylbutyrate (top), DMV (bottom). FIG. 5 illustrates representative Chromatograms of Low QC (0.6 ug/mL Sodium 2,2-Dimethylbutyrate) in Dog Plasma sodium 2,2-dimethylbutyrate (top), DMV (bottom). FIG. 6 illustrates representative Chromatograms of QC-Mid (10 ug/mL Sodium 2,2-Dimethylbutyrate) in Dog Plasma sodium 2,2-dimethylbutyrate (top), DMV (bottom). FIG. 7 illustrates representative Chromatograms of QC-High (40 ug/mL sodium 2,2-dimethylbutyrate) in Dog Plasma sodium 2,2-dimethylbutyrate (top), DMV (bottom).

### Example 2

### Analysis of Human Plasma

Sodium 2,2-dimethylbutyrate is currently in clinical development and human plasma samples collected from patients enrolled in clinical studies will require bioanalytical analysis for concentration determination of sodium 2,2-dimethylbutyrate using a validated method. The quantitative data obtained will be used to calculate the human pharmacokinetic parameters for subjects administered various dose levels of DMB in clinical studies. The objective of this study is to validate the LC-MS/MS method for the analysis of sodium 2,2-dimethylbutyrate in human plasma.

Sodium 2,2-dimethylbutyrate and the added internal standard, DMV, were extracted from human plasma using protein precipitation. The supernatant was dried, reconstituted, and derivatized to create benzyl amides of the analyte and internal standard. The resulting sample was dried and reconstituted for analysis by High Performance Liquid Chromatography (HPLC) on a reverse phase HPLC column. The analytes were detected and quantitated by Tandem Mass Spectrometry. Calibration was accomplished by a 1/x² weighted linear regression of the ratio of the peak areas of analyte to internal standard (sodium 2,2-dimethylbutyrate/DMV) to the corresponding nominal concentration sodium 2,2-dimethylbutyrate.

The following procedures describe requirements for the validation which were completed in this study: System Suitability, Specificity Linearity, Accuracy, Intra-day variation, Precision, Intra-day variation, Sensitivity, Lower Limit of Quantitation (LLOQ) Stability, Multiple freeze/thaw cycles in human plasma Bench-top stability in human plasma, Long term storage stability in human plasma Recovery from human plasma, and Dilution (10×).

Test Articles, internal standards, reagents and instrumentation used were as in Example 1. Sodium EDTA human plasma was obtained from Bioreclamation, Inc. (Hicksville, N.Y.). Dilutions were generally made as described below; however, weights and volumes of stock solutions may have varied. These changes are documented in the raw data. Miscellaneous Solutions, Mobile Phase Solutions and System Suitability Solutions used were as described for Example 1. Sodium 2,2-dimethylbutyrate and DMV solutions were prepared and stored as described above.

Control human plasma from six different lots was extracted according to the extraction procedure to evaluate the method specificity. Control human plasma from six different lots were fortified with internal standard and extracted according to the extraction procedure to evaluate the method specificity.

Preparation of Human Plasma Calibration Standards

The following aliquots of Working Solutions were added to 0.1 mL of human plasma to prepare 7 calibration standards.

| Concentration of Sodium | Sodium 2,2- | Fortified |
|---|---|---|
| 2,2-Dimethylbutyrate in | Dimethylbutyrate working | volume |
| Control (without Int. Std.) | none | none |
| Control + IS (with Int. Std.) | none | 10 |
| 0.20 | 2.0 | 10 |
| 0.40 | 4.0 | 10 |
| 1.0 | 10 | 10 |
| 4.0 | 40 | 10 |
| 10.0 | 100 | 10 |
| 20.0 | 200 | 10 |
| 50.0 | 500 | 10 |

Preparation of Human Plasma Qc Samples. Three Levels of Quality Control Samples (Qc-Low, QC-Mid and QC-High), at 0.60, 10 and 40 μg/mL Sodium 2,2-Dimethylbutyrate were prepared. Samples were also prepared at the Low Limit of Quantitation (LLOQ) at 0.20 μg/mL Sodium 2,2-Dimethylbutyrate. The following aliquots of sodium 2,2-dimethylbutyrate Working Stock Solutions were added to 0.1 mL of human plasma, and processed as fresh QC samples, or used for storage stability experiments.

| | Concentration | Sodium | |
|---|---|---|---|
| | of Sodium | 2,2-Dimethylbutyrate | |
| | 2,2-Dimethylbutyrate | QC working stock | Fortified |
| QC Sample | in plasma (ug/mL) | solution (ug/mL) | volume |
| LLOQ | 0.20 | 2.0 | 10 |
| QC-Low | 0.60 | 6.0 | 10 |
| QC-Middle | 10.0 | 100 | 10 |
| QC-High | 40.0 | 400 | 10 |

Triplicate QC-Low and QC-High samples were generated substituting water instead of plasma. These samples were analyzed during one of the validation runs and compared to 5 replicates of QC-Low and QC-High plasma samples.

A dilution QC sample (˜100 μg/mL sodium 2,2-dimethylbutyrate in human plasma) was prepared by fortifying an aliquot of 0.9029 mL human plasma with 0.0971 mL of the 1,029.79 ug/mL sodium 2,2-dimethylbutyrate QC Primary Stock Solution. Three-0.010 mL aliquots of the dilution QC sample were diluted with 0.090 mL control plasma to obtain a 10-fold dilution. These diluted plasma samples were fortified with internal standard and processed through the analytical procedure.

Control human plasma was thawed at ambient temperature or in tepid water. As needed, the control plasma was centrifuged ˜3,500 rpm for 5 minutes. An aliquot of 0.10 mL of plasma was transferred into individual centrifuge tubes. The 0.10 mL of plasma was fortified with 10 ul of working stock solution for the calibration curve standards and QC samples, respectively. The tubes were briefly mixed. All plasma samples, except the plasma control, were fortified with 10 ul of the 10.0 ug/mL DMV Working Stock Solution and briefly mixed. The control+IS sample and Dilution QCs were fortified with 10 ul Dilution Solution and briefly mixed. The control sample was fortified with 20 ul of the Dilution Solution and briefly mixed. An aliquot of 1.0 mL of acetonitrile was added to the tube followed by vortexing for 2 minutes. The tube was centrifuged at ˜14,000 rpm for 10 minutes. The supernatant was transferred to a 15 mL glass centrifuge tube. An aliquot of 10 μL of the 1.0 M sodium hydroxide solution was added to the tube. The supernatant was dried in the TurboVap at approximately 37° C. under a nitrogen flow. The residue was dissolved in 0.5 mL acetonitrile by vortexing for 5 seconds. Aliquots of 10 μL for both the 5% benzylamine solution and the 5% N,Ndiisopropylethylamine solution were added to the tube, and vortexed for 5 seconds. The sample was stored at −20° C. for approximately 20 minutes. An aliquot of 10 μL of the 60 mg/mL cooled Deoxo-Fluor solution was added to the tube and vortexed 5 seconds. The sample was stored at −20° C. for approximately 20 minutes. The extract was dried in the TurboVap at approximately 37° C. under a nitrogen flow. The dried extract was reconstituted in 1.0 mL of the Reconstitution Solution and vortexed 10 seconds. The tube was centrifuged ˜3,500 rpm for 5 minutes. The extract was transferred to an autosampler vial or 96-well plate for LCMS/MS analysis.

LC-MS/MS Conditions

The following LC-MS/MS conditions were applied for the analysis of Sodium 2,2-Dimethylbutyrate in human plasma and stock solution stability analysis (2 week and 1 month): HPLC Parameters: Column (Phenomenex Synergi RP Max 4 i, 150×2 mm, with a guard column cartridge or prefilter); Column flow rate (0.3 mL/min). The following details were added to the method beginning with the 1-month plasma stability analysis: The flow was increased to 0.4 mL/min after peak elution to ensure matrix removal from the column. The flow was diverted to waste before and after peak elution for some runs); Column temperature (ambient); injection volumes (1, 2, or 5 μL); Mobile Phase A (0.5% formic acid in water); Mobile Phase B (0.5% formic acid in acetonitrile); Mode (isocratic, 80% Mobile Phase B; run time (5 minutes)

The following LC-MS/MS conditions were applied for the analysis of underivatized sodium 2,2-dimethylbutyrate and DMV for stock solution stability analysis (2 to 3.5 month), and system suitability associated with these analyses:

HPLC Parameters:

| Column: | Phenomenex Luna C18(2) 5 i, 150 × 2 mm, | | |
|---|---|---|---|
| | with a guard column cartridge or prefilter | | |
| Column flow rate: | 0.3 mL/min. | | |
| Column temperature: | Ambient | | |
| Injection volume | 5 μL | | |
| Mobile Phase A: | 0.025% acetic acid in water | | |
| Mobile Phase B: | 0.025% acetic acid in acetonitrile | | |
| Gradient | Time (min.) | % A | % B |
| | 0 | 80 | 20 |
| | 1 | 30 | 70 |
| | 3 | 30 | 70 |
| | 3.1 | 80 | 20 |

| | |
|---|---|
| 7 | stop |

| Mass Spectrometer: | Applied Biosystems API 3000 | | |
|---|---|---|---|
| Ionization Interface: | TurboIon Spray (electrospray) | | |
| Ionization mode: | Negative | | |
| Transition Ion | | Precursor Ion | |
| Parameters: | Compound | Q1 Mass (amu) | Q3 Mass (amu) |
| | Sodium 2,2- | 115 | 115 |
| | DMV | 129 | 129 |

Calculations

The peak areas of sodium 2,2-dimethylbutyrate, and the internal standard DMV, were integrated by using the Analyst (Version 1.1) software provided by PE Sciex. The calibration curves were generated via least-square linear regression analysis. The general equation is as follows:

y=a+b*x

where, y=Peak area ratio (analyte area to internal standard area); x=Analyte calibration standard concentration, nominal; a=Intercept; b=Slope. All reported concentration data were calculated from 1/x² weighted linear regression curves.

The samples were analyzed on each of three days to determine precision, accuracy, linearity. System suitability solutions were analyzed prior to each sample set. One set of calibration curve mixed standards at the concentrations of 0.2, 0.4, 1.0, 4.0, 10, 20, and 50 μg/mL Sodium 2,2-Dimethylbutyrate in human plasma. LLOQ samples in five replicates at 0.2 μg/mL Sodium 2,2-Dimethylbutyrate in human plasma. QC-Low samples in five replicates at 0.6 μg/mL Sodium 2,2-Dimethylbutyrate in human plasma. QC-Mid samples in five replicates at 10 μg/mL Sodium 2,2-Dimethylbutyrate in human plasma. QC-High samples in five replicates at 40 μg/mL Sodium 2,2-Dimethylbutyrate in human plasma. One human plasma control sample (blank) and one human plasma control sample fortified with internal standard (zero). System suitability samples (n=6) containing sodium 2,2-dimethylbutyrate and DMV.

The following samples were also analyzed either in conjunction with one of the precision and accuracy runs or in one of the additional validation runs. Samples from six lots of control human plasma for specificity. Samples from six lots of control human plasma fortified with internal standard for specificity. Two concentration levels of unextracted QC-samples in triplicate (solvent standards) were analyzed for the evaluation of the recovery of sodium 2,2-dimethylbutyrate and DMV in human plasma. Two levels of QC samples (Low QC and High QC in triplicate) were subjected to three freeze/thaw cycles at approximately −70° C. prior to extraction to evaluate freeze/thaw stability. Two levels of QC samples (Low QC and High QC in triplicate) were placed on the bench top for approximately 17 hours prior to extraction for the evaluation of the bench top stability. Triplicate low QC samples and triplicate high QC samples were stored for one month and three months in a freezer at approximately −70° C., and then extracted and analyzed to evaluate long term stability in human plasma. Two levels of extracted QC samples (Low QC and High QC) were re-analyzed after approximately 71 hours in the autosampler at room temperature to evaluate the extract autosampler stability. Two levels of extracted QC samples (Low QC and High QC) were re-analyzed after approximately 8 hours in the freezer at approximately −20° C. to evaluate the extract freeze stability. Three aliquots of a dilution QC sample diluted 10-fold. Triplicate dilutions of the old stock solution (stored at approximately 4° C.) and new stock solutions prepared at intervals of two weeks and one month. Statistical calculations in the report tables were calculated from unrounded concentration values taken directly from the raw data. The concentration values were rounded for display purposes.

The system suitability was evaluated each day that human plasma validation samples were analyzed. One system suitability solution was injected six times. The precision for all system suitability analyses is shown in Table 9. The intra-day coefficient of variation percent (CV %) did not exceed 8.4% for sodium 2,2-dimethylbutyrate, and 10.6% for DMV. The LC-MS/MS method was found to be suitable for the validation.

The following samples were prepared and analyzed to evaluate specificity of the method. The chromatograms of these samples were evaluated for the presence of any interference peak at the retention time regions of sodium 2,2-dimethylbutyrate and DMV. Extracts of human control plasma from six different lots; Extracts of human control plasma from six different lots fortified with internal standard. The specificity samples contained sodium 2,2-dimethylbutyrate at a concentration ranging from 11% to 34% of the LLOQ. The same six plasma lots used for the specificity samples were reanalyzed, and the sodium 2,2-dimethylbutyrate concentration ranged from 9% to 19% of the LLOQ.

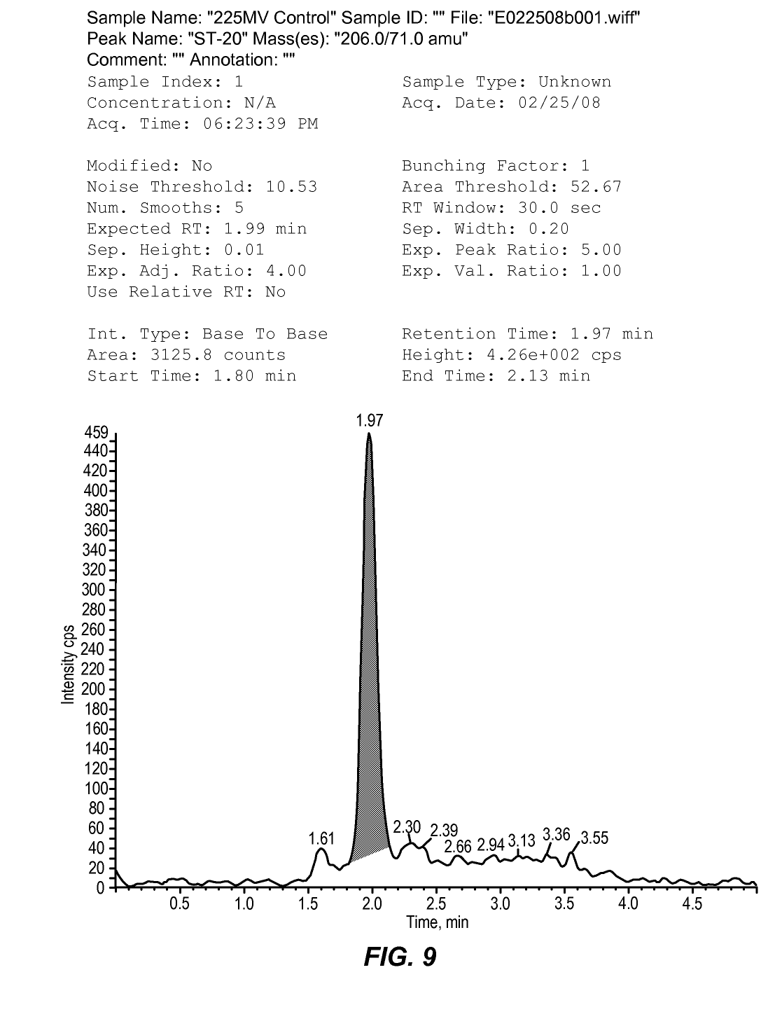

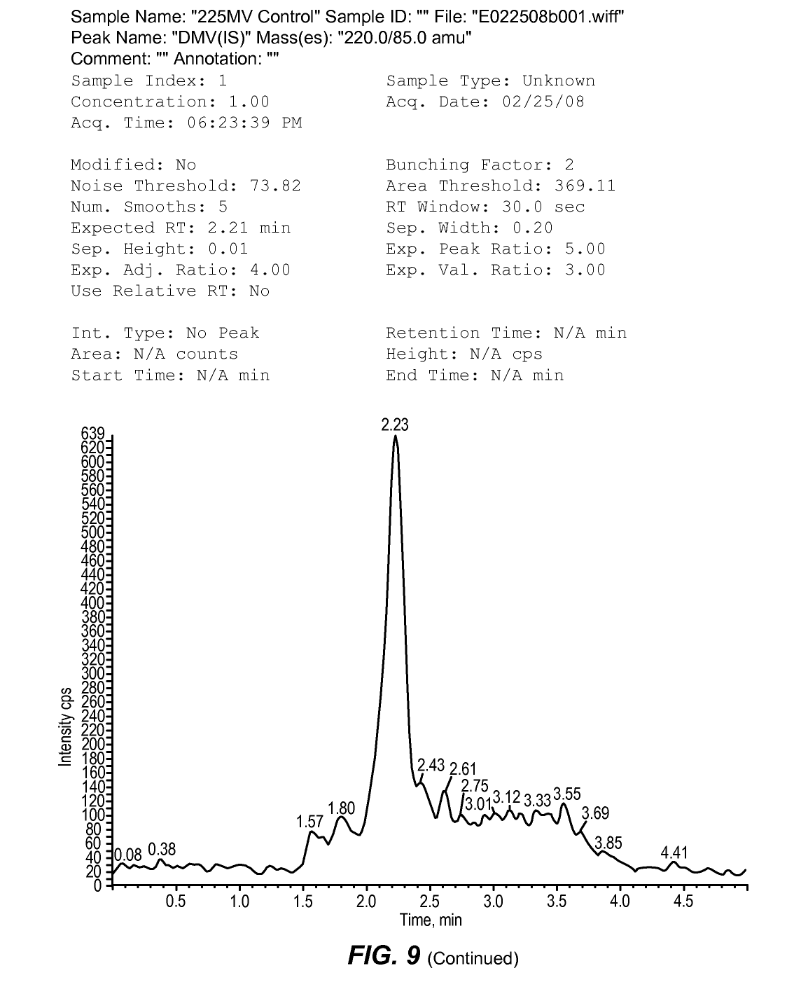

To determine the source of the sodium 2,2-dimethylbutyrate in the control plasma, water was substituted for plasma, and processed through the analytical procedure. Sodium 2,2-Dimethylbutyrate was found in the sample, similar to the level found in plasma controls. This indicates that the sodium 2,2-dimethylbutyrate levels found in control plasma are not related to the plasma, but can be considered background levels inherent in the method. Though the sodium 2,2-dimethylbutyrate background can vary, it is at a low level whereas quantitation is not affected. FIG. 9 is a representative chromatogram of a plasma control, which shows the sodium 2,2-dimethylbutyrate background levels.

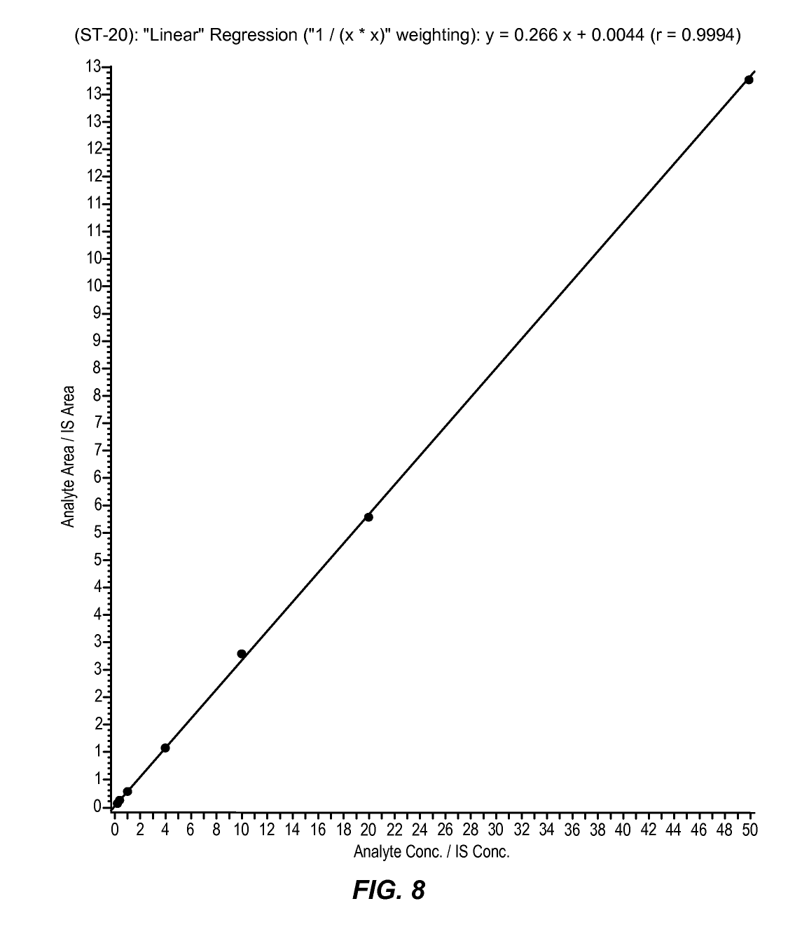

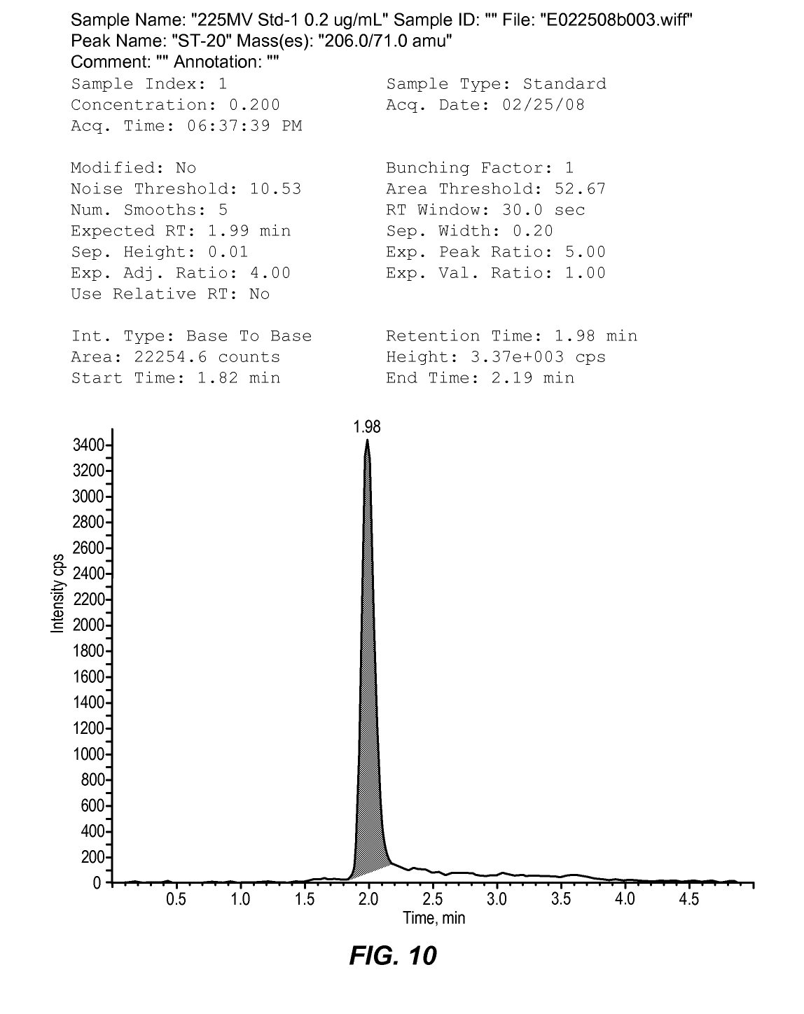

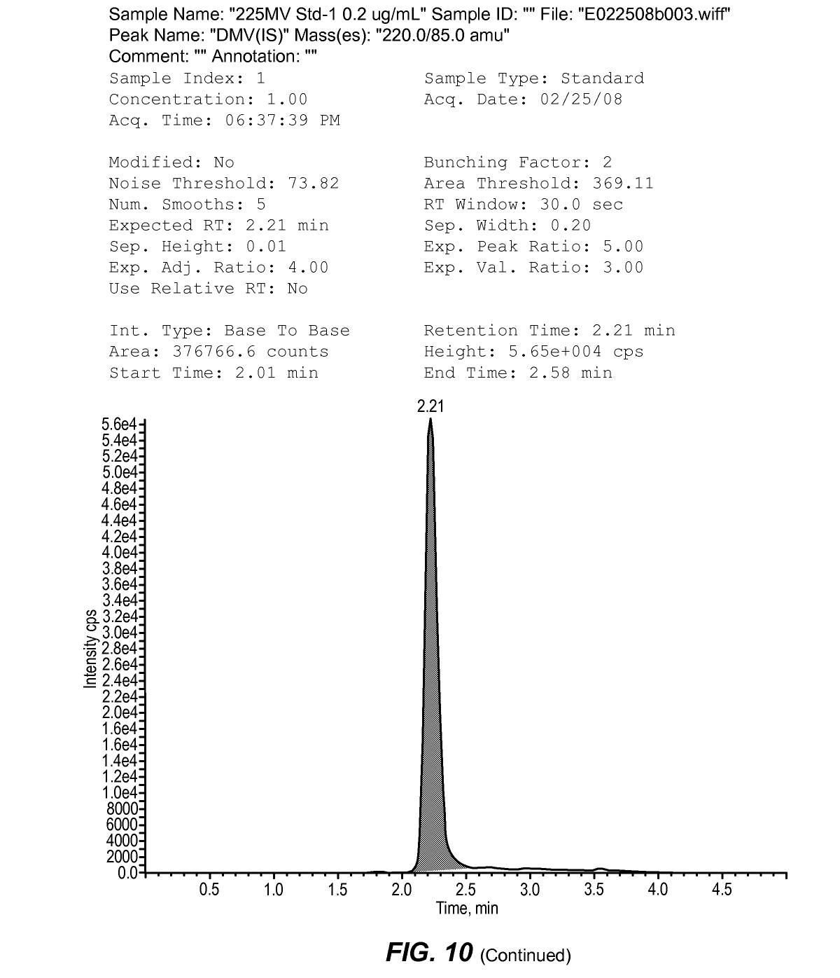

The relationship between the concentration of the analyte and the peak area ratios of the compound to internal standard was established. The parameters of the calibration curves for sodium 2,2-dimethylbutyrate are listed in Table 10. A typical calibration curve, depicted in FIG. 8, shows linearity for sodium 2,2-dimethylbutyrate over the concentration range of 0.20 μg/mL to 50 μg/mL. FIG. 10 is a representative chromatogram of a 0.20 μg/mL calibration standard. Correlation coefficients were >0.9949, satisfying the acceptance criteria of r≧0.990.

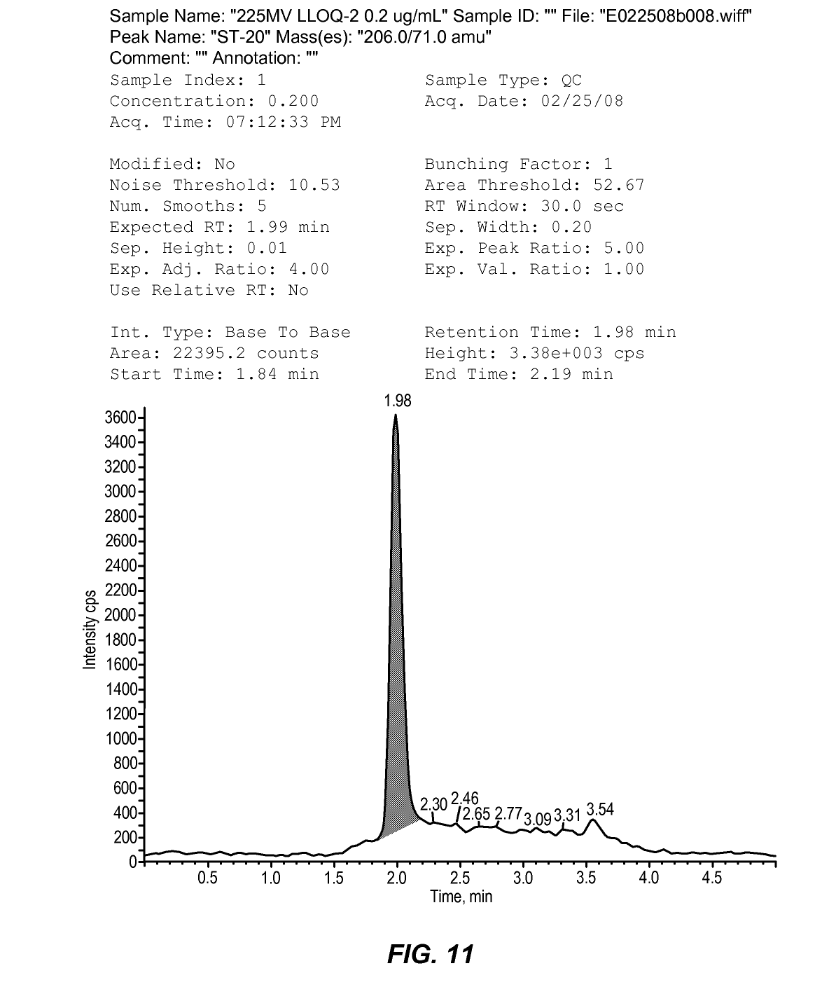

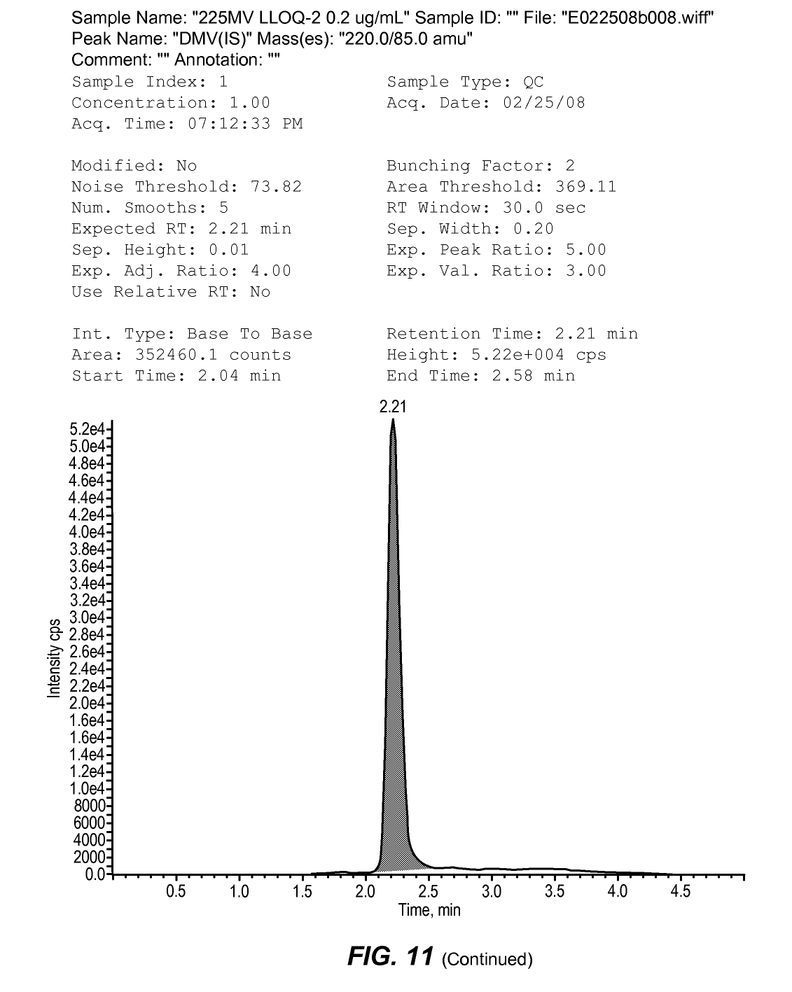

Back-calculated concentrations of QC samples (LLOQ, QC-Low, QC-Mid, and QC-High) for sodium 2,2-dimethylbutyrate were used for the statistical treatment of intra-day accuracy and precision. The data are shown in Table 11. FIGS. 11-14 contain representative chromatograms of the four QC levels.

Overall precision of the method was measured by the percent coefficient of variation (CV %). Table 11 shows the CV % for the LLOQ QCs ranging from 2.2% to 5.1%. The CV % range for the Low-, Mid-, and High-QCs was from 5.1% to 11.7%. These values are within the CV % acceptance limits of <20% for LLOQQCs and <15% for Low-, Mid-, and High-QCs.

Overall accuracy of the method was measured by the percent relative error (RE %), which was determined by comparing the mean values of the measured concentrations with the nominal concentrations of the analyte. Table 11 shows the RE % for the LLOQ QCs ranged from −0.3% to 9.0% The RE % range for the Low-, Mid-, and High-QCs was from −7.2% to 3.6%. Thus, all RE % values meet acceptance criteria (+20% for LLOQ-QCs and +15% for Low-, Mid-, and High-QCs).

The data indicate that the method provides good intra-day precision and accuracy over the LLOQ to QC-High range for sodium 2,2-dimethylbutyrate. Typical chromatograms of sodium 2,2-dimethylbutyrate in plasma samples are presented in FIGS. 9-14.

Three-day grand CV % and RE % values were used for the evaluation of the inter-day precision and accuracy. They were calculated from all the LLOQ, QC-Low, QC-Mid and QC-High sample data listed in Table 11. The grand CV % values range from 5.5% to 8.6%, and the grand RE % values range from −1.8% to 4.9%. The data, which fall within the acceptable limits of ≦15% (≦20% for LLOQ) for the CV % and ±15% (±20% for LLOQ) for the RE %, indicate that the method provides good inter-day precision and accuracy throughout the LLOQ to QC-High range.

The data for the LLOQ are presented in Table 11. The values of the three-day grand CV % and grand RE % are 553% and 4.9%, respectively. All values are well within the acceptable limits of ≦20% for CV and ±20% RE, indicating that the lower limit of quantitation for this method is 0.2 μg/mL for Sodium 2,2-Dimethylbutyrate.

The recovery was evaluated for sodium 2,2-dimethylbutyrate and DMV. This was determined by comparison of the peak areas of plasma QC samples at Low-QC and High-QC levels versus those of fortified water blank samples (water substituted for plasma) at the same concentration levels. The data are listed in Table 12. In many cases, the CV % was >15% for the five replicates of plasma QCs or the three replicates of fortified water blanks. The recovery experiment was repeated, and the data are listed in Table 12. Again, many CV % values were >15%. It is believed that the derivatization step in the procedure is the cause of this peak area variability. Therefore, the recovery obtained is an approximation. The recovery of sodium 2,2-dimethylbutyrate from human plasma ranged from 61.8% to 72.2%. The recovery of DMV from human plasma ranged from 65.3% to 75.7%.

The stability of sodium 2,2-dimethylbutyrate in human plasma was evaluated at approximately −70° C. for three cycles using QC-Low and QC-High samples in triplicate. The freeze time was at least 12 to 24 hours, with a minimum thaw time of one hour. The results are shown in Table 13. The CV % values for the QC-Low and QC-High stability samples are 3.3% and 9.8%, respectively. To calculate the RE %, the measured mean concentration was compared to the nominal concentration. The RE % values for the QC-Low and QC-High stability samples are 2.3% and −5.5%, respectively. All CV % and RE % values fall within the limits of <15% and ±15%, respectively, indicating that sodium 2,2-dimethylbutyrate is stable in human plasma after three freeze/thaw cycles.

Bench top stability of sodium 2,2-dimethylbutyrate in plasma was evaluated at room temperature for 22.5 hours. Triplicate QC-Low and QC-High samples were extracted and analyzed after these storage conditions. The results are shown in Table 14. The CV % values for the QC-Low and QC-High stability samples are 7.8% and 2.9%, respectively. To calculate the RE %, the measured mean concentration was compared to the nominal concentration. The RE % values for the QC-Low and QC-High stability samples are 11.8% and −3.4%, respectively. All CV % and RE % values fall within the limits of ≦15% and ±15%, respectively, indicating that sodium 2,2-dimethylbutyrate is stable in human plasma after ambient bench top storage for 22.5 hours.

After extraction, the QC samples were analyzed and left in the autosampler for at least 71 hours, then re-injected onto the LC-MS/MS. Triplicate QC-Low and QC-High samples were analyzed for the autosampler stability determination. The data are listed in Table 15. The CV % values for the QC-Low and QC-High stability samples are 7.5% and 6.4%, respectively. To calculate the RE %, the measured mean concentration was compared to the nominal concentration. The RE % values for the QC-Low and QC-High stability samples are 1.9% and 4.6%, respectively. All CV % and RE % values fall within the limits of ≦15% and ±15%, respectively, indicating that Sodium 2,2-Dimethylbutyrate is stable in the extract after at least 71 hours of ambient storage in the autosampler

QC-Low and QC-High extract samples in triplicate were analyzed and then stored in a −20° C. freezer for approximately 8 hours. These QC samples were then re-analyzed to evaluate the extract freezer stability. The data are presented in Table 16. The CV % values for the QC-Low and QC-High stability samples are 8.6% and 1.2%, respectively. To calculate the RE %, the measured mean concentration was compared to the nominal concentration. The RE % values for the QC-Low and QC-High stability samples are −6.3% and −13.0%, respectively. All CV % and RE % values fall within the limits of ≦15% and ±15%, respectively, indicating that sodium 2,2-dimethylbutyrate is stable in the extract after at least 8 hours of storage in the freezer at −20° C.

QC-Low and QC-High plasma samples, which had been stored in a freezer at approximately −70° C. for one month and three months, were extracted in triplicate and analyzed. The CV % values for the QC-Low and QC-High stability samples range from 4.0% to 11.1%. To calculate the RE %, the measured mean concentration was compared to the nominal concentration. The RE % values for the QC-Low and QC-High stability samples range from −13.7% to 4.1%. All CV % values and RE % values are within the acceptance criteria (≦15% and ±15% respectively). Therefore, sodium 2,2-dimethylbutyrate can be considered stable in human plasma at approximately −70° C. for up to three months

Fresh stock solutions of sodium 2,2-dimethylbutyrate were prepared at intervals of two weeks and 1, 2, 3 and 3.5 months after the initial standard preparation. Similarly, the stability intervals for DMV were two weeks and 1, 2 and 3 months. The initial (or old) stock solutions and the fresh stock solutions were diluted into the calibration standard range and analyzed. The peak areas for the fresh (new) and old standard solutions were compared.

The results are listed in Table 18. Beginning with the 2-month stability analysis, sodium 2,2-dimethylbutyrate and DMV stock solutions were analyzed underivatized. This was done since the peak areas of both sodium 2,2-dimethylbutyrate and DMV varied among replicate derivatized preparations. Analyzing the standard solutions underivatized was a simpler procedure which gave more accurate and precise results

For sodium 2,2-dimethylbutyrate, all the CV values in Table 18 are ≦15%. The sodium 2,2-dimethylbutyrate RE % values for all the stability intervals are ≦15%. The data shows that sodium 2,2-dimethylbutyrate stock solutions are stable for at least 3.5 months when stored in a refrigerator at approximately 4° C.

For DMV, the RE % ranged from 14.3% to 23.9% for the stability intervals through 3 months. It was suspected that the original DMV stock solution concentration was higher than intended, since the stability comparisons were consistently lower for the 2 week through the three month intervals. The two month stability interval was repeated, by comparing a new DMV stock solution to the 1 month stock solution. The RE % for this comparison was −7.2%, which met the acceptance criteria of RE %≦15%. The data shows that DMV stock solutions are stable for at least 2 months when stored in a refrigerator at approximately 4° C.

A quality control sample was prepared at a concentration of 100 μg/mL Sodium 2,2-Dimethylbutyrate in human plasma. The QC sample was diluted 10-fold in three replicates with control plasma to obtain a concentration of sodium 2,2-dimethylbutyrate within the calibration range. The data from this analysis are presented in Table 19.

During the study, the calibration curve weighting factor was changed from 1/x to 1/x². The data generated from the three validation runs were calculated using both 1/x to 1/x². The data was subjected to Goodness of Fit calculations, which determines the sum of the squared residuals for the calibration curve standards. The weighting factor 1/x² was shown to be the best weighting factor. All quantitation data in the study was calculated using the weighting factor of 1/x². This deviation did not adversely affect the study since the data met the acceptance criteria.

During the study, the final reconstitution volume was changed from 0.2 mL to 1.0 mL. This improved the calibration curve linearity. Also, if sodium 2,2-dimethylbutyrate was not completely soluble in the reconstitution solution at higher concentration levels, increasing the reconstitution volume would have aided analyte solubility. This change was added to the study when the 1-month plasma stability samples were analyzed. This deviation had no adverse effect on the study, since calibration curve linearity improved.

The protocol specified a HPLC column flow of 0.3 mL/min throughout the run. A modification was added whereby after the analyte and internal standard eluted, the column flow was increased from 0.3 to 0.4 mL/min. This extra solvent flush was added to the method as a precaution so uneluted matrix does not build up in the column during lengthy sample runs. This change was added to the study when the 1-month plasma stability samples were analyzed. This deviation had no adverse effect on the study, since the data met the acceptance criteria.

According to the COA for sodium 2,2-dimethylbutyrate, the standard was to be stored under ambient conditions in a desiccator. The protocol incorrectly listed storage under refrigerated conditions in a desiccator. During the study, the sodium 2,2-dimethylbutyrate neat standard was stored under frozen conditions (−20° C.) in a desiccator. This deviation had no impact on the study. Several weighings of sodium 2,2-dimethylbutyrate were previously made for stock solution stability analyses. The sodium 2,2-dimethylbutyrate stock solutions were found to be stable for 3.5 months, therefore, the neat standard must also be stable under frozen storage conditions.

For the specificity experiment, Sodium 2,2-Dimethylbutyrate was present in some controls at levels greater than 20% of the LLOQ level. This was a protocol deviation which specified that levels of sodium 2,2-dimethylbutyrate in plasma controls should be less than 20% of the LLOQ. This deviation had little effect on the study since the sodium 2,2-dimethylbutyrate background levels were at a low enough level that it did not interfere with the calibration curve and QCs.

The CV % and RE % values for the dilution QC experiment were 0.9% and −1.5%, respectively. The data, which fall within acceptance limits for CV % (≦15%) and RE % (±15%), indicate that using a 10-fold dilution yields analytical results that are precise and accurate.

According to the COA for sodium 2,2-dimethylbutyrate, the standard was to be stored under ambient conditions in a desiccator. The protocol incorrectly listed storage under refrigerated conditions in a desiccator. During the study, the sodium 2,2-dimethylbutyrate neat standard was stored under frozen conditions (−20° C.) in a desiccator. This deviation had no impact on the study. Several weighings of Sodium 2,2-Dimethylbutyrate were previously made for stock solution stability analyses. The sodium 2,2-dimethylbutyrate stock solutions were found to be stable for 3.5 months, therefore, the neat standard must also be stable under frozen storage conditions.

For the specificity experiment, sodium 2,2-dimethylbutyrate was present in some controls at levels greater than 20% of the LLOQ level. This was a protocol deviation which specified that levels of sodium 2,2-dimethylbutyrate in plasma controls should be less than 20% of the LLOQ. This deviation had little effect on the study since the sodium 2,2-dimethylbutyrate background levels were at a low enough level that it did not interfere with the calibration curve and QCs.

The method presented here for the determination of sodium 2,2-dimethylbutyrate in human plasma shows acceptable linearity, precision and accuracy for the calibration range of 0.2 μg/mL to 50 μg/mL. The method is specific for the internal standard, DMV, but did not meet the specificity criteria for sodium 2,2-dimethylbutyrate, since sodium 2,2-dimethylbutyrate was detected in blank plasma at a level up to 28% of the LLOQ concentration. It is believed that the sodium 2,2-dimethylbutyrate levels found in blank plasma are not related to the plasma, but can be considered background levels inherent in the method. The human plasma can be diluted 10-fold and analyzed with acceptable precision and accuracy. At concentration levels within the calibration range, sodium 2,2-dimethylbutyrate in human plasma is stable at room temperature on the bench top for at least 17 hours, and for three freeze/thaw cycles at approximately −70° C. Sodium 2,2-Dimethylbutyrate is stable in human plasma for at least 99 days when stored at approximately −70° C. The recovery in human plasma ranged from 61.8% to 72.2% for sodium 2,2-dimethylbutyrate, and 65.3% to 75.7% for DMV. These are approximate recovery ranges since the CV % values were high due to the variability introduced by the derivatization step.

TABLE 9
System Suitability

| | Sodium 2,2- | DMV |
|---|---|---|
| Extraction | Dimethylbutyrate | Peak Area |
| First | 91660.8 | 292598.4 |
| Extraction | 92501.2 | 294329.1 |
| | 86846.3 | 303973.8 |
| | 87735.7 | 299042.2 |
| | 88699.3 | 278210.1 |
| | 90952.9 | 285735.7 |
| Mean | 89732.7 | 292314.9 |
| SD | 2291.6 | 9247.2 |
| CV % | 2.6 | 3.2 |
| Second | 60597.5 | 198014.1 |
| Extraction | 65722.8 | 189355.4 |
| | 62424.9 | 192388.6 |
| | 63523.9 | 187220.6 |
| | 58020.1 | 181053.9 |
| | 55561.0 | 173814.8 |
| Mean | 60975.0 | 186974.6 |
| SD | 3723.6 | 8545.7 |
| CV % | 6.1 | 4.6 |
| Third | 92064.6 | 286577.3 |
| Extraction | 91204.2 | 287827.3 |
| | 104325.8 | 313156.2 |
| | 96076.9 | 283821.1 |
| | 91922.3 | 295427.8 |
| | 94025.7 | 278795.8 |
| Mean | 94936.6 | 290934.3 |
| SD | 4930.6 | 12170.3 |
| CV % | 5.2 | 4.2 |
| Fourth | 87951.8 | 291639.7 |
| Extraction | 76377.4 | 222422.3 |
| | 80500.9 | 245366.0 |
| | 79054.0 | 244551.5 |
| | 70034.9 | 222565.2 |
| | 71688.5 | 233433.2 |
| Mean | 77601.3 | 243329.7 |
| SD | 6501.7 | 25712.7 |
| CV % | 8.4 | 10.6 |
| | Sodium 2,2- | d-14 Sodium 2,2- |
| | Dimethylbutyrate | Dimethylbutyrate |
| Extraction | Peak Area | Peak Area |
| Fifth | 145435.5 | 130788.5 |
| Extraction | 140396.6 | 127749.8 |
| | 143536.7 | 130014.3 |
| | 143968.0 | 128912.5 |
| | 142459.5 | 129740.0 |
| | 143886.9 | 128994.5 |
| Mean | 143280.5 | 129366.6 |
| SD | 1705.3 | 1052.6 |
| CV % | 1.2 | 0.8 |
| Sixth | 32638.6 | 112985.0 |
| Extraction | 28642.2 | 85739.8 |
| | 28017.4 | 85252.5 |
| | 29175.9 | 98513.8 |
| | 29378.6 | 90227.5 |
| | 27709.0 | 93923.5 |
| Mean | 29260.3 | 94440.4 |
| SD | 1775.8 | 10379.1 |
| CV % | 6.1 | 11.0 |
| Seventh | 19459.1 | 41198.7 |
| Extraction | 20498.9 | 42397.6 |
| | 20207.3 | 41923.2 |
| | 20064.5 | 40865.8 |
| | 21067.1 | 40672.6 |
| | 22077.0 | 43973.4 |
| Mean | 20562.3 | 41838.6 |
| SD | 910.4 | 1232.3 |
| CV % | 4.4 | 2.9 |
| Eight | 906702.1 | 278491.9 |
| Extraction | 954476.6 | 287278.4 |
| | 977318.3 | 316891.8 |
| | 976855.3 | 308049.4 |
| | 956517.7 | 326391.7 |
| | 1002666.0 | 318021.4 |
| Mean | 962422.7 | 305854.1 |
| SD | 32411.4 | 18922.1 |
| CV % | 3.4 | 6.2 |

TABLE 10
Standard Curve Parameters for Sodium
2,2-Dimethylbutyrate Weighted (1/x²)

| | | | | Correlation |
|---|---|---|---|---|
| Extraction | n | Intercept | Slope | Coefficient |
| First | 7 | 0.0131 | 0.236 | 0.9971 |
| Second | 7 | 0.0044 | 0.266 | 0.9994 |
| Third | 7 | 0.00605 | 0.29 | 0.9980 |
| Fourth | 6 | 0.0142 | 0.265 | 0.9947 |
| Fifth | 7 | 0.0371 | 0.251 | 0.9954 |

TABLE 11
Accuracy and Precision of Sodium 2,2-Dimethylbutyrate
in QC Samples for Human Plasma

| | LLOQ | QC-Low | QC-Mid | QC-High |
|---|---|---|---|---|
| Concentration (μg/mL) | 0.20 | 0.60 | 10.0 | 40.0 |
| Day-1 | 0.210 | 0.541 | 11.5 | 44.1 |
| | 0.203 | 0.630 | 11.5 | 38.2 |
| | 0.207 | 0.677 | 8.90 | 41.0 |
| | 0.189 | 0.593 | 9.77 | 38.4 |
| | 0.188 | 0.627 | 9.40 | 38.1 |
| Mean | 0.199 | 0.614 | 10.2 | 40.0 |
| SD | 0.010 | 0.050 | 1.2 | 2.6 |
| CV % | 5.1 | 8.2 | 11.7 | 6.6 |
| RE % | −0.3 | 2.3 | 2.0 | −0.1 |
| Day-2 | 0.220 | 0.586 | 9.21 | 38.1 |
| | 0.222 | 0.649 | 10.6 | 41.0 |
| | 0.212 | 0.553 | 10.1 | 39.1 |
| | 0.222 | 0.650 | 10.5 | 39.6 |
| | 0.214 | 0.605 | 10.8 | 43.7 |
| Mean | 0.218 | 0.609 | 10.3 | 40.3 |
| SD | 0.005 | 0.042 | 0.6 | 2.2 |
| CV % | 2.2 | 6.9 | 6.3 | 5.4 |
| RE % | 9.0 | 1.4 | 2.5 | 0.7 |
| Day-3 | 0.213 | 0.627 | 9.66 | 39.8 |
| | 0.202 | 0.577 | 9.14 | 33.2¹ |
| | 0.204 | 0.665 | 8.83 | 34.1 |
| | 0.229 | 0.605 | 9.73 | 36.6 |
| | 0.212 | 0.634 | 10.0 | 37.9 |
| Mean | 0.212 | 0.622 | 9.5 | 37.1 |
| SD | 0.011 | 0.033 | 0.5 | 2.4 |
| CV % | 5.0 | 5.3 | 5.1 | 6.5 |
| RE % | 6.0 | 3.6 | −5.2 | −7.2 |
| Three-day Mean | 0.210 | 0.615 | 9.98 | 39.3 |
| Three-day SD | 0.012 | 0.040 | 0.85 | 2.64 |
| Grand CV % | 5.5 | 6.4 | 8.6 | 6.7 |
| Grand RE % | 4.9 | 2.4 | −0.2 | −1.8 |

TABLE 12
Recovery of Sodium 2,2-Dimethylbutyrate and
DMV from Human Plasma at the Two QC Levels

| | Peak Area | | | |
|---|---|---|---|---|
| | QC-Low | | QC-High | |
| | Sodium 2,2- | DMV | Sodium 2,2- | DMV |
| Plasma QC | 31111.1 | 193910.2 | 2511462.1 | 247742.0 |
| | 50070.3 | 282469.6 | 2720990.8 | 249267.2 |
| | 32914.2 | 216943.7 | 2628006.8 | 252477.6 |
| | 57690.9 | 325262.3 | 2160481.5 | 204646.8 |
| | 29858.5 | 180390.0 | 2278298.5 | 195538.5 |
| Mean | 40329.0 | 239795.2 | 2459847.9 | 229934.4 |
| SD | 12707.4 | 61810.9 | 235434.4 | 27484.6 |
| CV % | 31.5 | 25.8 | 9.6 | 12.0 |
| Fortified Water | 38546.9 | 199504.9 | 2987309.2 | 247841.4 |
| Blank | 41337.5 | 214118.5 | 3065624.2 | 238315.0 |
| | 33230.0 | 168829.0 | 2225340.0 | 177641.7 |
| Mean | 37704.8 | 194150.8 | 2759424.5 | 221266.0 |
| SD | 4118.8 | 23114.6 | 464185.3 | 38078.9 |
| CV % | 10.9 | 11.9 | 16.8 | 17.2 |
| Recovery % | 107 | 124 | 89 | 104 |
| Plasma QC | 30120.3 | 160024.0 | 2087900.6 | 180491.7 |
| | 33523.9 | 193159.7 | 2078423.5 | 215747.4 |
| | 51942.9 | 260961.4 | 1606381.6 | 162141.2 |
| | 69711.5 | 383440.4 | 2926105.4 | 274944.6 |
| | 51016.6 | 268358.5 | 2324136.3 | 211232.2 |
| Mean | 47263.0 | 253188.8 | 2204589.5 | 208911.4 |
| SD | 15988.8 | 85934.8 | 480172.8 | 43033.8 |
| CV % | 33.8 | 33.9 | 21.8 | 20.6 |
| Fortified Water | 36597.0 | 176726.0 | —¹ | —¹ |
| Blank | 37067.3 | 182152.2 | —¹ | —¹ |
| | 43257.6 | 217818.0 | —¹ | —¹ |
| Mean | 38974.0 | 192232.1 | —¹ | —¹ |
| SD | 3717.2 | 22323.6 | —¹ | —¹ |
| CV % | 9.5 | 11.6 | —¹ | —¹ |
| Recovery % | 121 | 132 | —¹ | —1 |

TABLE 13
Freeze/Thaw Cycle Stability at approximately −70°
C. for Sodium 2,2-Dimethylbutyrate in Human Plasma

Sodium 2,2-Dimethylbutyrate

| | QC-Low | QC-High |
|---|---|---|
| Concentration (μg/mL) | 0.60 | 40.0 |
| | 0.593 | 36.8 |
| | 0.633 | 34.7 |
| | 0.615 | 41.9 |
| Mean | 0.614 | 37.8 |
| SD | 0.020 | 3.7 |
| CV % | 3.3 | 9.8 |
| RE % | 2.3 | −5.5 |

TABLE 14
Bench Top Stability for Sodium 2,2-
Dimethylbutyrate in Human Plasma

Sodium 2,2-Dimethylbutyrate

| | QC-Low | QC-High |
|---|---|---|
| Concentration (μg/mL) | 0.60 | 40.0 |
| | 0.651 | 37.5 |
| | 0.730 | 38.8 |
| | 0.631 | 39.7 |
| Mean | 0.671 | 38.7 |
| SD | 0.052 | 1.1 |
| CV % | 7.8 | 2.9 |
| RE % | 11.8 | −3.4 |

TABLE 15
Extract Autosampler Stability for Sodium
2,2-Dimethylbutyrate in Human Plasma

Sodium 2,2-Dimethylbutyrate

| | QC-Low | QC-High |
|---|---|---|
| Concentration (μg/mL) | 0.60 | 40.0 |
| | 0.575 | 43.8 |
| | 0.596 | 38.8 |
| | 0.663 | 42.9 |
| Mean | 0.611 | 41.8 |
| SD | 0.046 | 2.7 |
| CV % | 7.5 | 6.4 |
| RE % | 1.9 | 4.6 |

TABLE 16
Extract Freezer (−20° C.) Stability for
Sodium 2,2-Dimethylbutyrate in Human Plasma

Sodium 2,2-Dimethylbutyrate

| | QC-Low | QC-High |
|---|---|---|
| Concentration (μg/mL) | 0.60 | 40.0 |
| | 0.618 | 35.2 |
| | 0.532 | 34.7 |
| | 0.537 | 34.4 |
| Mean | 0.562 | 34.8 |
| SD | 0.048 | 0.4 |
| CV % | 8.6 | 1.2 |
| RE % | −6.3 | −13.0 |

TABLE 17
Long-Term Freezer Stability at approximately −70°
C. for Sodium 2,2-Dimethylbutyrate in Human Plasma

Sodium 2,2-Dimethylbutyrate

| | | |
|---|---|---|
| | QC-Low | QC-High |
| Concentration (μg/mL) | 0.60 | 40.0 |

1 Month Interval

| | | |
|---|---|---|
| | 0.572 | 36.5 |
| | 0.601 | 39.5 |
| | 0.484 | 38.6 |
| Mean | 0.552 | 38.2 |
| SD | 0.061 | 1.5 |
| CV % | 11.0 | 4.0 |
| RE % | −7.9 | −4.6 |

3 Month Interval

| | | |
|---|---|---|
| | 0.632 | 37.5 |
| | 0.655 | 30.2 |
| | 0.586 | 35.7 |
| Mean | 0.624 | 34.5 |
| SD | 0.035 | 3.8 |
| CV % | 5.6 | 11.1 |
| RE % | 4.1 | −13.7 |

TABLE 18
Sodium 2,2-Dimethylbutyrate Stock Solution
Stability at approximately 4° C.

| | Sodium 2,2-Dimethylbutyrate | | DMV (IS) | |
|---|---|---|---|---|
| | Peak Area | Peak Area | Peak Area | Peak Area |
| Replicate | Old | New | Old | New |

2 Week Interval

| | | | | |
|---|---|---|---|---|
| 1 | 858662.7 | 1077223.7 | 99038.2 | 85008.8 |
| 2 | 938260.0 | 884179.0 | 94898.3 | 71809.5 |
| 3 | 887239.2 | 976301.6 | 84544.0 | 81910.6 |
| Mean | 894720.6 | 979234.8 | 92826.8 | 79576.3 |
| Std Dev | 40322.6 | 96555.8 | 7465.8 | 6902.3 |
| CV % | 4.5 | 9.9 | 8.0 | 8.7 |
| RE % (to New) | −8.6 | — | 16.7 | — |

1 Month Interval

| | | | | |
|---|---|---|---|---|
| 1 | 136547.2 | 113545.0 | 54202.2 | 36908.2 |
| 2 | 140023.5 | 107495.7 | 54892.8 | 34980.9 |
| 3 | 123225.5 | 141547.8 | 46654.5 | 53863.9 |
| Mean | 133265.4 | 120862.8 | 51916.5 | 41917.7 |
| Std Dev | 8866.8 | 18167.3 | 4570.1 | 10390.5 |
| CV % | 6.7 | 15.0 | 8.8 | 24.8 |
| RE % (to New) | 10.3 | — | 23.9 | — |

2 Month Interval

| | | | | |
|---|---|---|---|---|
| 1 | 1001978.2 | 1022925.3 | 330008.4 | 292123.8 |
| 2 | 933408.5 | 933308.0 | 310299.6 | 283125.9 |
| 3 | 985345.5 | 902580.7 | 326355.1 | 270377.1 |
| Mean | 973577.4 | 952938.0 | 322221.0 | 281875.6 |
| Std Dev | 35767.5 | 62527.7 | 10484.6 | 10927.1 |
| CV % | 3.7 | 6.6 | 3.3 | 3.9 |
| RE % (to New) | 2.2 | — | 14.3 | — |

3 Month Interval

| | | | | |
|---|---|---|---|---|
| 1 | 696124.6 | 737643.8 | 269196.2 | 243036.5 |
| 2 | 746641.8 | 636579.8 | 271617.6 | 206686.8 |
| 3 | 704955.6 | 739564.3 | 280571.2 | 247619.6 |
| Mean | 715907.3 | 704596.0 | 273795.0 | 232447.6 |
| Std Dev | 26980.6 | 58911.6 | 5991.9 | 22426.9 |
| CV % | 3.8 | 8.4 | 2.2 | 9.6 |
| RE % (to New) | 1.6 | | 17.8 | |

~3.5 Month Interval (Sodium 2,2-dimethylbutyrate) ~2
Month Interval DMV (IS)

| | | | | |
|---|---|---|---|---|
| 1 | 461707.1 | 464705.0 | 1336637.9 | 1382355.3 |
| 2 | 493334.6 | 463725.2 | 1413804.8 | 1475571.0 |
| 3 | 442481.3 | 500307.8 | 1362989.5 | 1576011.5 |
| Mean | 465841.0 | 476246.0 | 1371144.1 | 1477979.3 |
| Std Dev | 25677.4 | 20843.9 | 39224.4 | 96850.6 |
| CV % | 5.5 | 4.4 | 2.9 | 6.6 |
| RE % (to New) | −2.2 | | −7.2 | |

TABLE 19
10-Fold Dilution QC Samples for Sodium
2,2-Dimethylbutyrate in Human Plasma

| | Sodium 2,2- |
|---|---|
| Concentration (μg/mL) | 100 |
| | 109 |
| | 114 |
| | 114 |
| Mean | 112 |
| SD | 2.7 |
| CV % | 2.4 |
| RE % | 12.5 |

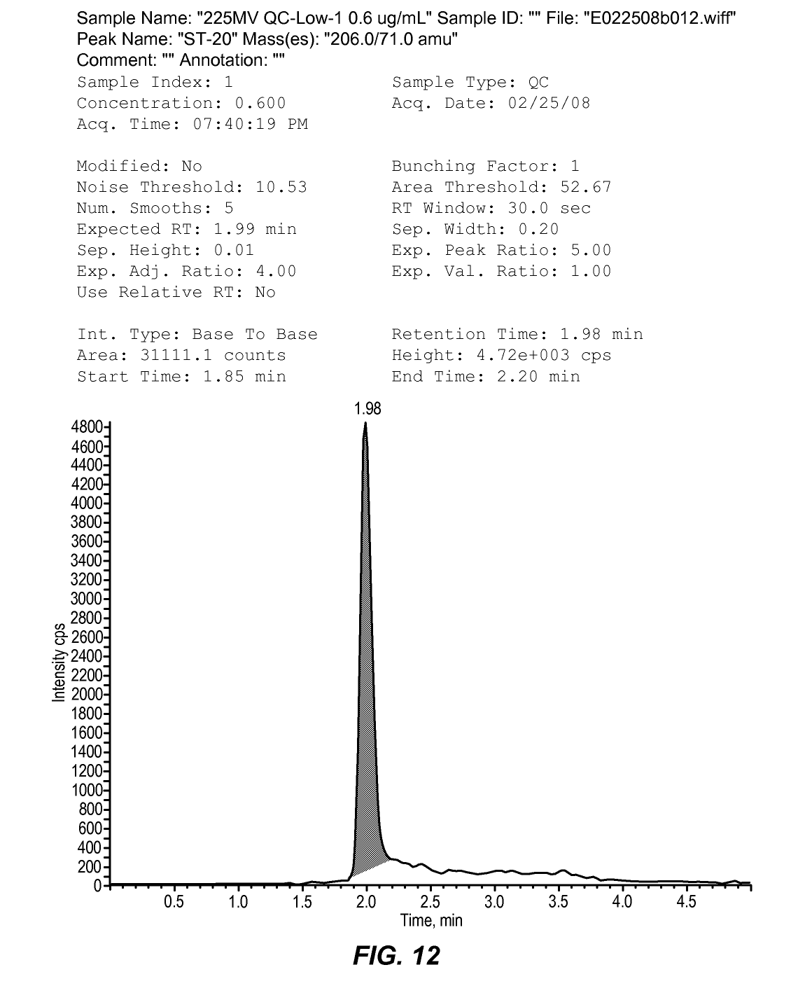

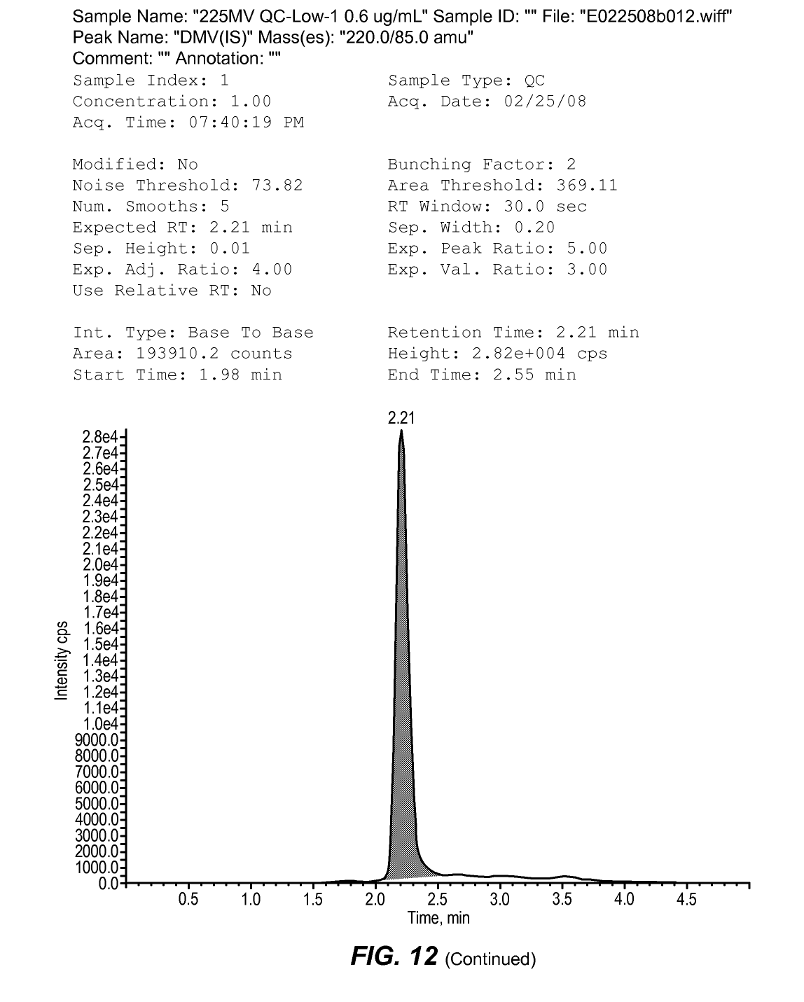

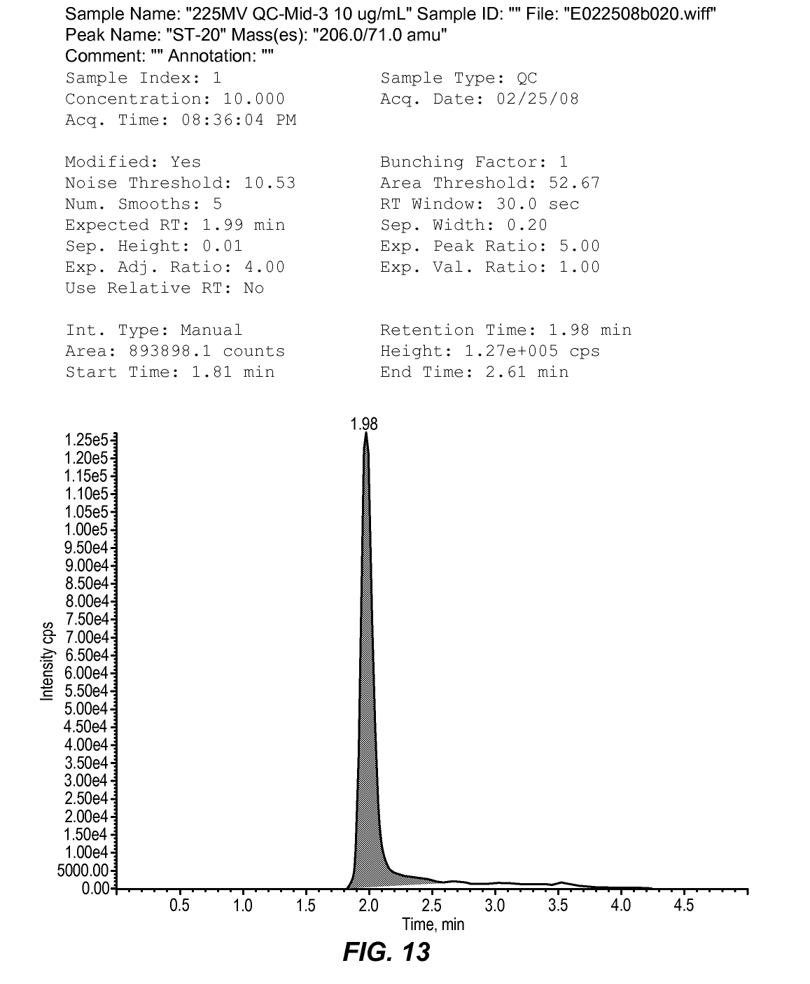

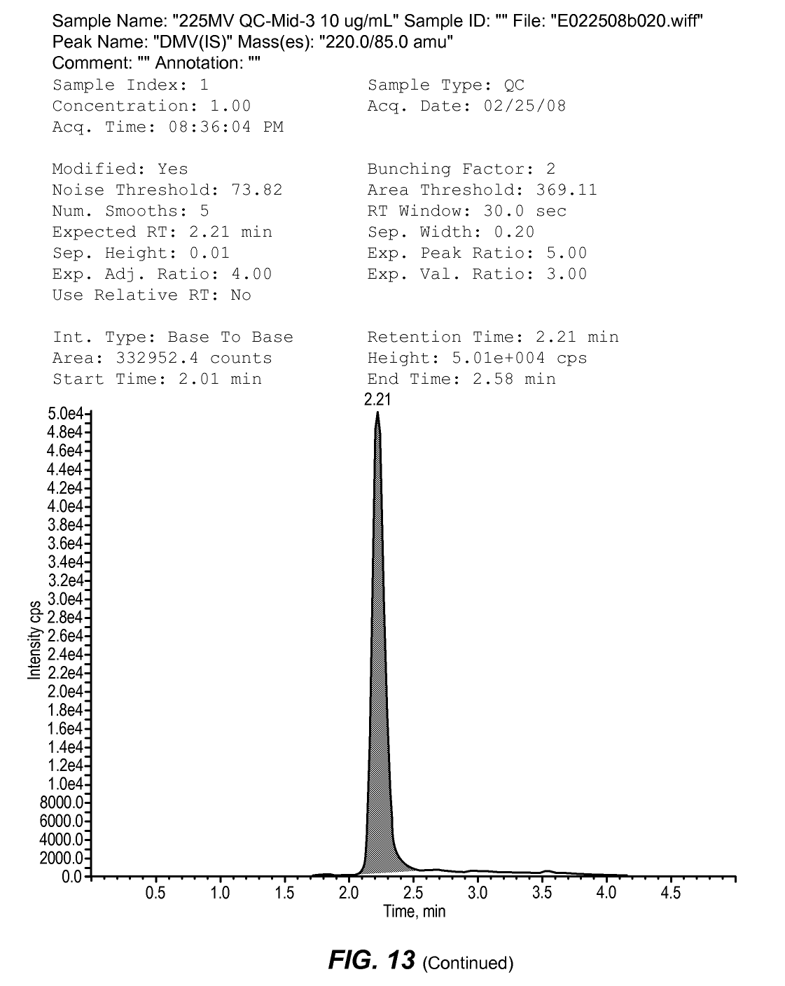

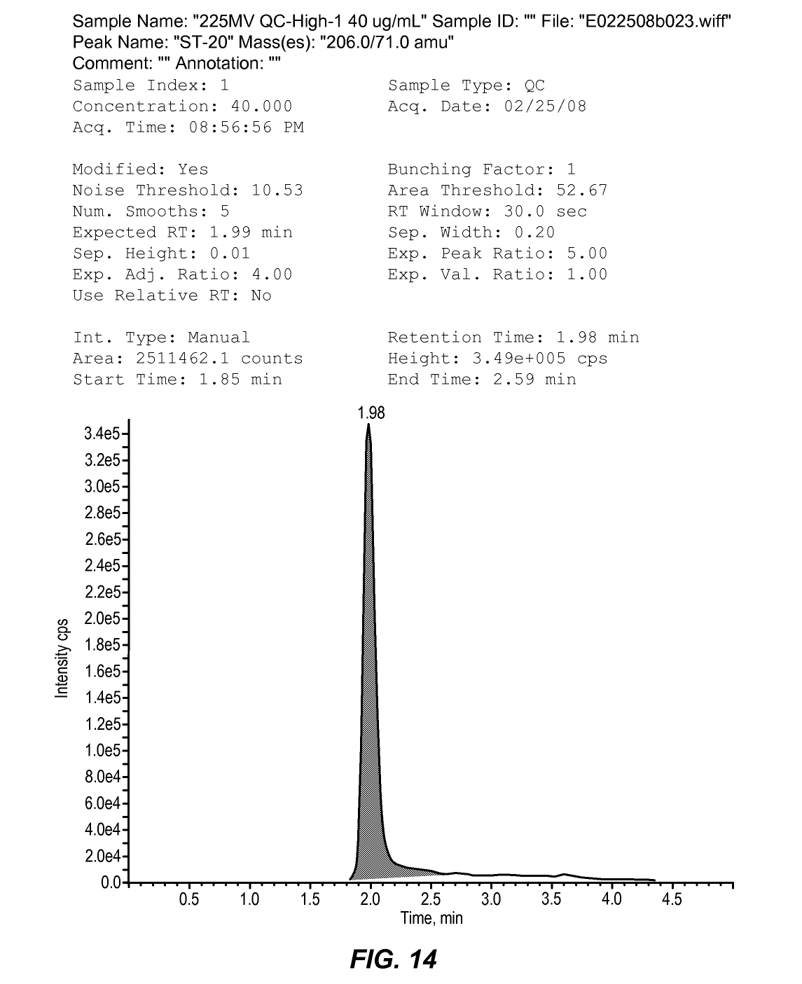

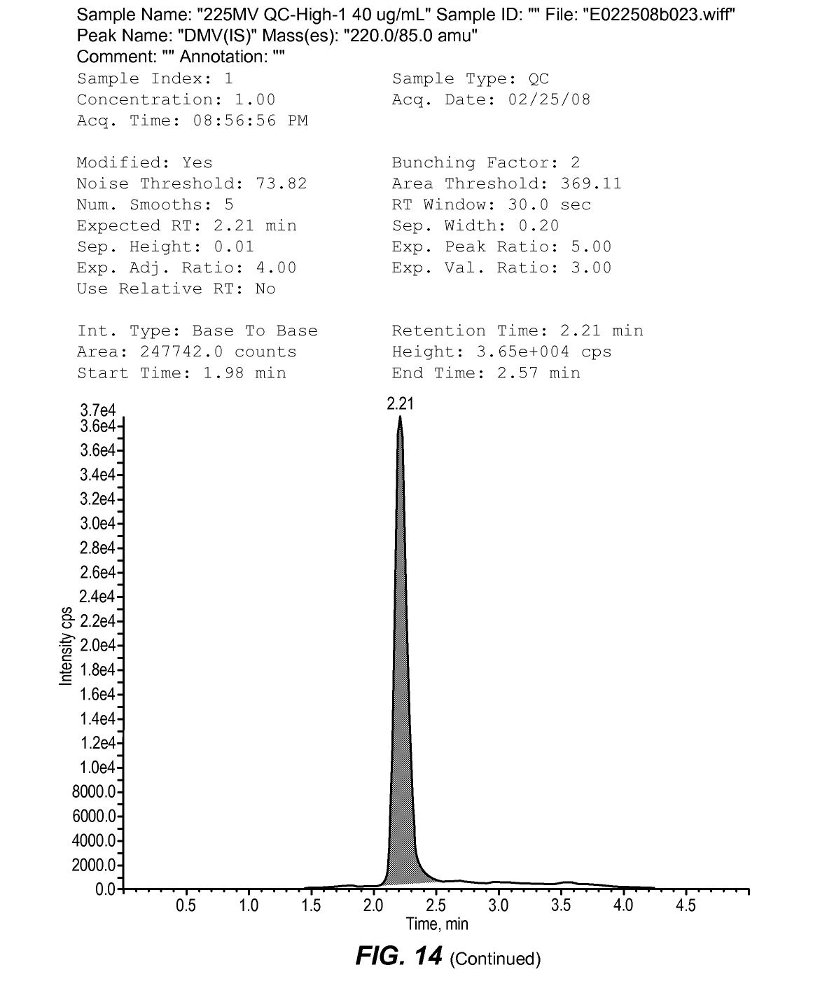

Representative chromatograms from the study are illustrated in FIGS. 8-14. FIG. 8 illustrates representative Calibration Curve. FIG. 9 illustrates representative Chromatograms of Control Human Plasma sodium 2,2-dimethylbutyrate (top), DMV (bottom). FIG. 10 illustrates representative Chromatograms of Standard-1 (0.2 μg/mL sodium 2,2-dimethylbutyrate) in Human Plasma sodium 2,2-dimethylbutyrate (top), DMV (bottom). FIG. 11 illustrates representative Chromatograms of LLOQ (0.2 ug/mL sodium 2,2-dimethylbutyrate) in Human Plasma sodium 2,2-dimethylbutyrate (top), DMV (bottom). FIG. 12 illustrates representative Chromatograms of Low QC (0.6 ug/mL sodium 2,2-dimethylbutyrate) in Human Plasma sodium 2,2-dimethylbutyrate (top), DMV (bottom). FIG. 13 illustrates representative Chromatograms of QC-Mid (10 ug/mL sodium 2,2-dimethylbutyrate) in Human Plasma sodium 2,2-dimethylbutyrate (top), DMV (bottom). FIG. 14 illustrates representative Chromatograms of QC-High (40 ug/mL sodium 2,2-dimethylbutyrate) in Human Plasma sodium 2,2-dimethylbutyrate (top), DMV (bottom).

### Example 3

### Analysis of Rat Plasma

Test Articles, internal standards, reagents and instrumentation used were as in Example 1. Sodium EDTA rat plasma was obtained from Bioreclamation, Inc. (Hicksville, N.Y.). Dilutions were generally made as described below; however, weights and volumes of stock solutions may have varied. These changes are documented in the raw data. Miscellaneous Solutions, Mobile Phase Solutions and System Suitability Solutions used were as described for Example 1. Sodium 2,2-dimethylbutyrate and DMV solutions were prepared and stored as described above.

Extracts of Control Plasma: Control rat plasma from six different lots were extracted according to the extraction procedure to evaluate the method specificity. Extracts of Control Plasma Fortified with Internal Standard: Control rat plasma from six different lots were fortified with internal standard and extracted according to the extraction procedure to evaluate the method specificity.

Preparation of Rat Plasma Calibration Standards

The following aliquots of Working Solutions were added to 0.1 mL of rat plasma to prepare 7 calibration standards.

| Concentration of Sodium | Sodium 2,2- | Fortified |
|---|---|---|
| 2,2-Dimethylbutyrate in | Dimethylbutyrate working | volume |
| Control (without Int. Std.) | none | none |
| Control + IS (with Int. Std.) | none | 10 |
| 0.20 | 2.0 | 10 |
| 0.40 | 4.0 | 10 |
| 1.0 | 10 | 10 |
| 4.0 | 40 | 10 |
| 10.0 | 100 | 10 |
| 20.0 | 200 | 10 |
| 50.0 | 500 | 10 |

Preparation of Rat Plasma QC Samples

Three levels of Quality Control samples (QC-Low, QC-Mid and QC-High), at 0.60, 10 and 40 μg/mL sodium 2,2-dimethylbutyrate were prepared. Samples were also prepared at the Low Limit of Quantitation (LLOQ) at 0.20 μg/mL sodium 2,2-dimethylbutyrate. The following aliquots of sodium 2,2-dimethylbutyrate Working Stock Solutions were added to 0.1 mL of rat plasma, and processed as fresh QC samples, or used for storage stability experiments.

| | Concentration of | Sodium 2,2- | Fortified |
|---|---|---|---|
| QC Sample | Sodium 2,2- | Dimethylbutyrate QC | volume |
| LLOQ | 0.20 | 2.0 | 10 |
| QC-Low | 0.60 | 6.0 | 10 |
| QC-Middle | 10.0 | 100 | 10 |
| QC-High | 40.0 | 400 | 10 |

Triplicate QC-Low and QC-High samples were generated substituting water instead of plasma. These samples were analyzed during one of the validation runs and compared to 5 replicates of QC-Low and QC-High plasma samples.

A dilution QC sample (˜100 μg/mL sodium 2,2-dimethylbutyrate in rat plasma) was prepared by fortifying an aliquot of 0.9029 mL rat plasma with 0.0971 mL of the 1,029.79 ug/mL sodium 2,2-dimethylbutyrate QC Primary Stock Solution. Three-0.010 mL aliquots of the dilution QC sample were diluted with 0.090 mL control plasma to obtain a 10-fold dilution. These diluted plasma samples were fortified with internal standard and processed through the analytical procedure.

Control rat plasma was thawed at ambient temperature or in tepid water. As needed, the control plasma was centrifuged ˜3,500 rpm for 5 minutes. An aliquot of 0.10 mL of plasma was transferred into individual centrifuge tubes. The 0.10 mL of plasma was fortified with 10 ul of working stock solution for the calibration curve standards and QC samples, respectively. The tubes were briefly mixed. All plasma samples, except the plasma control, were fortified with 10 ul of the 10.0 ug/mL DMV Working Stock Solution and briefly mixed. The control+IS sample and Dilution QCs were fortified with 10 ul Dilution Solution and briefly mixed. The control sample was fortified with 20 ul of the Dilution Solution and briefly mixed. An aliquot of 1.0 mL of acetonitrile was added to the tube followed by vortexing for 2 minutes. The tube was centrifuged at ˜14,000 rpm for 10 minutes. The supernatant was transferred to a 15 mL glass centrifuge tube. An aliquot of 10 μL of the 1.0 M sodium hydroxide solution was added to the tube. The supernatant was dried in the TurboVap at approximately 37° C. under a nitrogen flow. The residue was dissolved in 0.5 mL acetonitrile by vortexing for 5 seconds. Aliquots of 10 μL for both the 5% benzylamine solution and the 5% N,Ndiisopropylethylamine solution were added to the tube, and vortexed for 5 seconds. The sample was stored at −20° C. for approximately 20 minutes. An aliquot of 10 μL of the 60 mg/mL cooled Deoxo-Fluor solution was added to the tube and vortexed 5 seconds. The sample was stored at −20° C. for approximately 20 minutes. The extract was dried in the TurboVap at approximately 37° C. under a nitrogen flow. The dried extract was reconstituted in 1.0 mL of the Reconstitution Solution and vortexed 10 seconds. The tube was centrifuged ˜3,500 rpm for 5 minutes. The extract was transferred to an autosamper vial or 96-well plate for LCMS/MS analysis.

LC-MS/MS Conditions

The following LC-MS/MS conditions were applied for the analysis of Sodium 2,2-Dimethylbutyrate in rat plasma:

HPLC Parameters:

| | |
|---|---|
| Column: | Phenomenex Synergi RP Max 4 ì, 150 × 2 mm, |
| | with a guard column cartridge or prefilter |
| Column flow rate: | 0.3 mL/min. |
| | The flow was increased to 0.4 mL/min after |
| | peak elution to ensure matrix removal from |
| | the column. The flow was diverted to waste |
| | before and after peak elution for some runs. |
| Column temperature: | Ambient |
| Injection volumes | 2 or 5 μL |
| used in the study: | |
| Mobile Phase A: | 0.5% formic acid in water |
| Mobile Phase B: | 0.5% formic acid in acetonitrile |
| Mode: | Isocratic, 80% Mobile Phase B |
| Run time: | 5 minutes |

Mass Spectrometry Parameters:

| Mass Spectrometer: | Applied Biosystems API 3000 | | |
|---|---|---|---|
| Ionization Interface: | TurboIon Spray (electrospray) | | |
| Ionization mode: | Positive | | |
| Transition | | Precursor Ion | |
| Ion Parameters: | Compound | Q1 Mass (amu) | Q3 Mass (amu) |
| | Sodium 2,2- | 206 | 71 |
| | DMV | 220 | 85 |

Calculations

The peak areas of sodium 2,2-dimethylbutyrate, and the internal standard DMV, were integrated by using the Analyst (Version 1.1) software provided by PE Sciex. The calibration curves were generated via least-square linear regression analysis. The general equation is as follows:

y=a+b*x

where, y=Peak area ratio (analyte area to internal standard area); x=Analyte calibration standard concentration, nominal; a=Intercept; b=Slope. All reported concentration data were calculated from 1/x2 weighted linear regression curves.

The samples were analyzed in one day to determine precision, accuracy, linearity. System suitability solutions were analyzed prior to each sample set. One set of calibration curve mixed standards at the concentrations of 0.2, 0.4, 1.0, 4.0, 10, 20, and 50 μg/mL sodium 2,2-dimethylbutyrate in rat plasma. LLOQ samples in five replicates at 0.2 μg/mL sodium 2,2-dimethylbutyrate in rat plasma. QC-Low samples in five replicates at 0.6 μg/mL sodium 2,2-dimethylbutyrate in rat plasma. QC-Mid samples in five replicates at 10 μg/mL sodium 2,2-dimethylbutyrate in rat plasma. QC-High samples in five replicates at 40 μg/mL sodium 2,2-dimethylbutyrate in rat plasma. One rat plasma control sample (blank) and one rat plasma control sample fortified with internal standard (zero).

System suitability samples (n=6) containing sodium 2,2-dimethylbutyrate and DMV.

For the remaining validation tests, a calibration curve and triplicates QCs at the low, mid and high levels were analyzed with each sample set. The following samples were also analyzed either in conjunction with one of the precision and accuracy runs or in one of the additional validation runs: Samples from six lots of control rat plasma for specificity; Samples from six lots of control rat plasma fortified with internal standard for specificity; Two concentration levels of unextracted QC-samples in triplicate (solvent standards) were analyzed for the evaluation of the recovery of sodium 2,2-dimethylbutyrate and DMV in rat plasma; Two levels of QC samples (Low QC and High QC in triplicate) were subjected to three freeze/thaw cycles at approximately −70° C. prior to extraction to evaluate freeze/thaw stability; Two levels of QC samples (Low QC and High QC in triplicate) were placed on the bench top for approximately 17 hours prior to extraction for the evaluation of the bench top stability; Triplicate low QC samples and triplicate high QC samples were stored for one month and three months in a freezer at approximately −70° C., and then extracted and analyzed to evaluate long term stability in rat plasma; Three aliquots of a dilution QC sample diluted 10-fold.

Statistical calculations in the report tables were calculated from unrounded concentration values taken directly from the raw data. The concentration values appearing in the report tables were rounded for display purposes.

The system suitability was evaluated each day that rat plasma validation samples were analyzed. One system suitability solution was injected six times. The precision for all system suitability analyses is shown in Table 20. The intra-day coefficient of variation percent (CV %) did not exceed 10.5% for Sodium 2,2-Dimethylbutyrate, and 10.8% for DMV. The LC-MS/MS method was found to be suitable for the validation.

The following samples were prepared and analyzed to evaluate specificity of the method. Chromatograms of these samples were evaluated for the presence of any interference peak at the retention time regions of sodium 2,2-dimethylbutyrate and DMV: Extracts of rat control plasma from six different lots; Extracts of rat control plasma from six different lots fortified with internal standard.

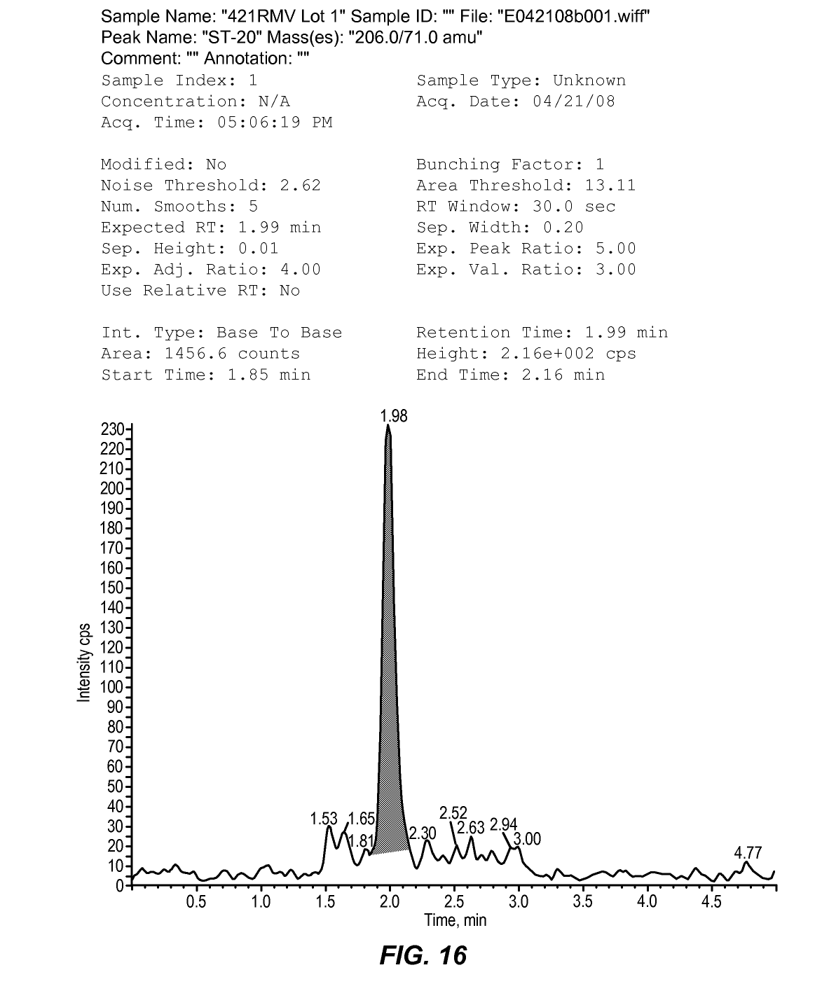

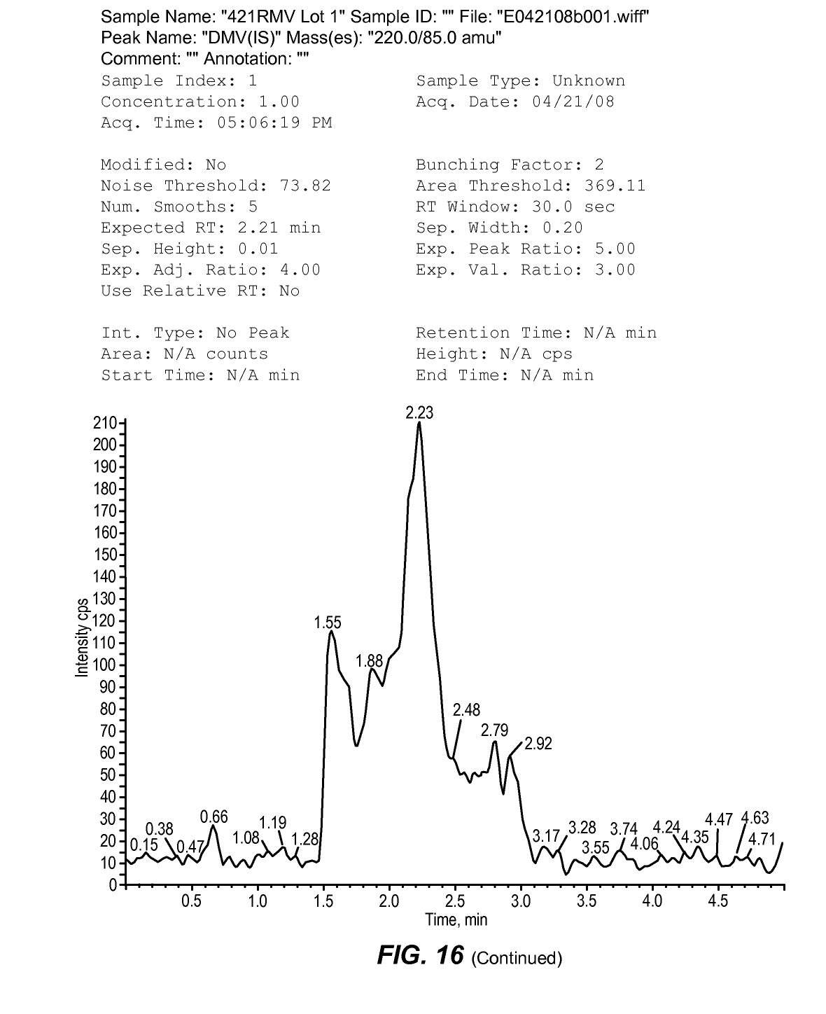

The specificity samples contained apparent sodium 2,2-dimethylbutyrate at a concentrations ranging from 13% to 28% of the LLOQ. The sodium 2,2-dimethylbutyrate peak in the specificity samples was not due to injector carryover. Similar, apparent levels of sodium 2,2-dimethylbutyrate in control plasma were observed during the full method validation. It was determined from experiments during the full method validation that the sodium 2,2-dimethylbutyrate levels found in control plasma are not related to the plasma, but can be considered background levels inherent in the method. Though the sodium 2,2-dimethylbutyrate background can vary, it is at a low level where quantitation is not affected. FIG. 16 is a representative chromatogram of a plasma control, which shows the sodium 2,2-dimethylbutyrate background levels.

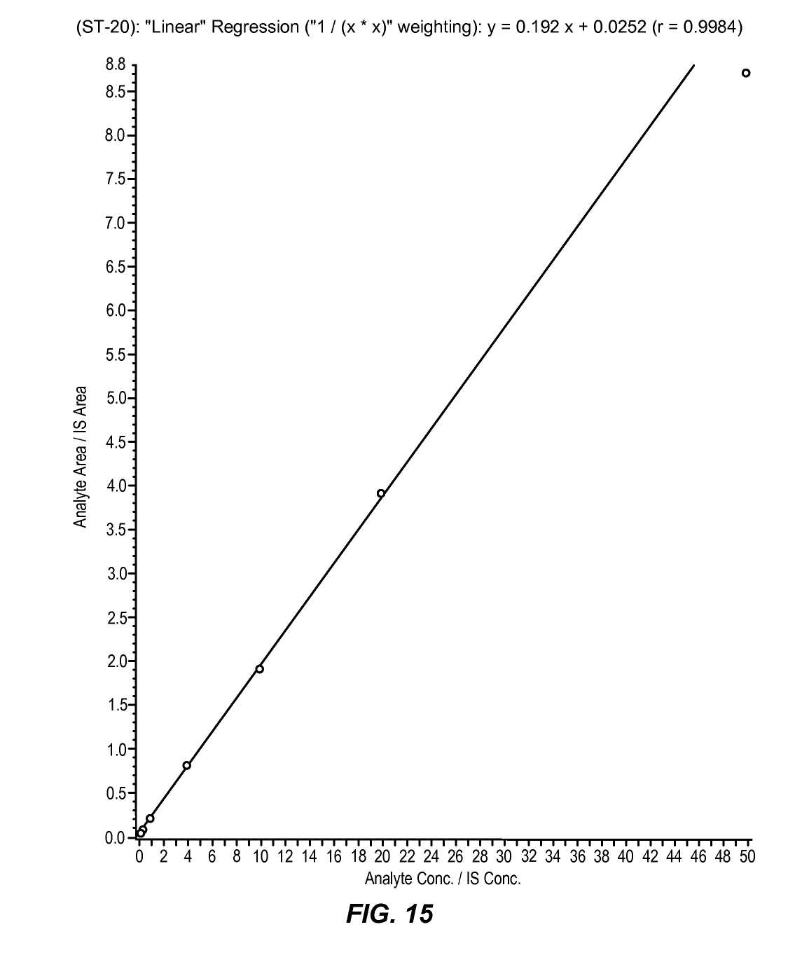

The relationship between the concentration of the analyte and the peak area ratios of the compound to internal standard was established. The parameters of the calibration curves for sodium 2,2-dimethylbutyrate are listed in Table 21. A typical calibration curve, depicted in FIG. 15, shows linearity for sodium 2,2-dimethylbutyrate over the concentration range of 0.20 μg/mL to 50 μg/mL. Correlation coefficients were >0.9949, satisfying the acceptance criteria of r≧0.990.

Back-calculated concentrations of QC samples (LLOQ, QC-Low, QC-Mid, and QC-High) for sodium 2,2-dimethylbutyrate were used for the statistical treatment of intra-day accuracy and precision. The data are shown in Table 22.

Overall precision of the method was measured by the percent coefficient of variation (CV %). Table 22 shows the CV % for the LLOQ QC was 5.3%. The CV % range for the Low-, Mid-, and High-QCs was from 1.9% to 4.9%. These values are within the CV % acceptance limits of <20% for LLOQQCs and <15% for Low-, Mid-, and High-QCs.

Overall accuracy of the method was measured by the percent relative error (RE %), which was determined by comparing the mean values of the measured concentrations with the nominal concentrations of the analyte. Table 22 shows the RE % for the LLOQ QC was −2.8%. The RE % range for the Low-, Mid-, and High-QCs was from 2.4% to 7.2%. Thus, all RE % values meet acceptance criteria (+20% for LLOQ-QCs and +15% for Low-, Mid-, and High-QCs).

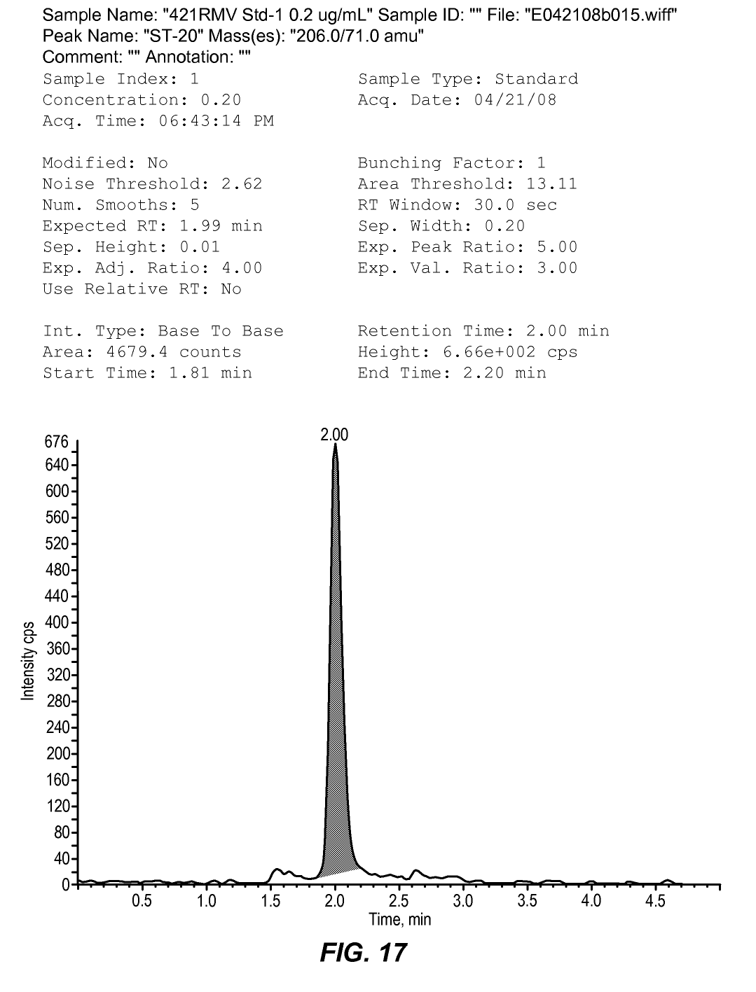

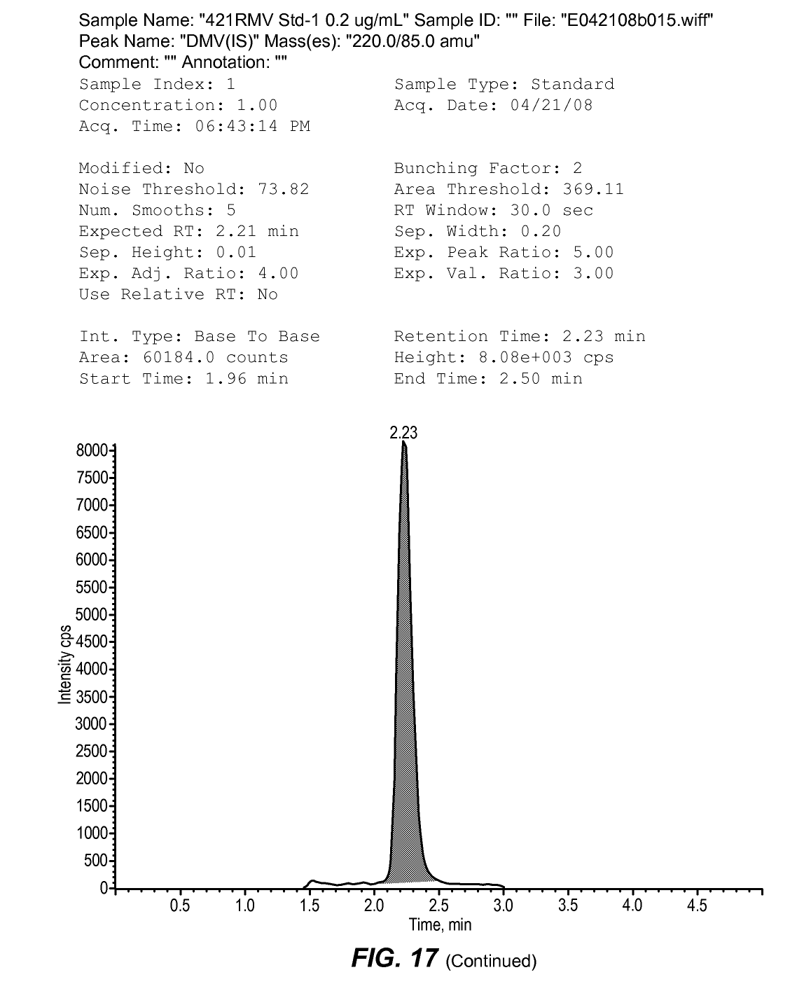

The data indicate that the method provides good intra-day precision and accuracy over the LLOQ to QC-High range for sodium 2,2-dimethylbutyrate. Typical chromatograms of sodium 2,2-dimethylbutyrate in plasma samples are presented in FIGS. 17-21.

The data for the LLOQ are presented in Table 22. The values of the CV % and RE % are 5.9% and −2.8%, respectively. All values are well within the acceptable limits of ≦20% for CV and ±20% RE, indicating that the lower limit of quantitation for this method is 0.2 μg/mL for sodium 2,2-dimethylbutyrate.

The recovery was evaluated for sodium 2,2-dimethylbutyrate and DMV. This was determined by comparison of the peak areas of plasma QC samples at Low-QC and High-QC levels versus those of fortified water blank samples (water substituted for plasma) at the same concentration levels. The data are listed in Table 23. In many cases, the CV % was >15% for the five replicates of plasma QCs or the three replicates of fortified water blanks. It is believed that the derivatization step in the procedure is the cause of this peak area variability. Therefore, the recovery obtained is an approximation. The recovery of sodium 2,2-dimethylbutyrate from rat plasma ranged from 46.5% to 62.0%. The recovery of DMV from rat plasma ranged from 50.2% to 67.6%.

The stability of sodium 2,2-dimethylbutyrate in rat plasma was evaluated at approximately −70° C. for three cycles using QC-Low and QC-High samples in triplicate. The freeze time was at least 12-24 hours, with a minimum thaw time of one hour. The results are shown in Table 24. The CV % values for the QC-Low and QC-High stability samples are 13.3% and 4.6%, respectively. To calculate the RE %, the measured mean concentration was compared to the nominal concentration.

The RE % values for the QC-Low and QC-High stability samples are −4.1% and 3.1%, respectively. All CV % and RE % values fall within the limits of ≦15% and ±15%, respectively, indicating that sodium 2,2-dimethylbutyrate is stable in rat plasma after three freeze/thaw cycles.

Bench top stability was evaluated at room temperature for approximately 12.5 hours. Triplicate QC-Low and QC-High samples were extracted and analyzed after these storage conditions. The results are shown in Table 25. The CV % values for the QC-Low and QC-High stability samples are 6.3% and 11.7%, respectively. To calculate the RE %, the measured mean concentration was compared to the nominal concentration. The RE % values for the QC-Low and QC-High stability samples are 7.6% and 9.1%, respectively. All CV % and RE % values fall within the limits of ≦15% and ±15%, respectively, indicating that sodium 2,2-dimethylbutyrate is stable in rat plasma after ambient bench top storage for approximately 12.5 hours.

QC-Low and QC-High plasma samples, which had been stored in a freezer at approximately −70° C. for one month and three months (98 days), were extracted in triplicate and analyzed. The data is presented in Table 26. At the one month interval, the RE % was −24.1% for the QC-Low level. This may have been an anomaly since at the QC-Low level for the three month interval, the RE % was 4.1%. All CV % values and RE % values at the 98 day interval were within the acceptance criteria (≦15% and ±15% respectively). Therefore, sodium 2,2-dimethylbutyrate can be considered stable in rat plasma at approximately −70° C. for at least 99 days.

A quality control sample was prepared at a concentration of 100 μg/mL sodium 2,2-dimethylbutyrate in rat plasma. The QC sample was diluted 10-fold in three replicates with control plasma to obtain a concentration of sodium 2,2-dimethylbutyrate within the calibration range. The data from this analysis are presented in Table 27.

The CV % and RE % values for the dilution QC experiment were 5.4% and −0.5%, respectively. The data, which fall within acceptance limits for CV % (≦15%) and RE % (±15%), indicate that using a 10-fold dilution yields analytical results that are precise and accurate.

According to the COA for sodium 2,2-dimethylbutyrate, the standard was to be stored under ambient conditions in a desiccator. The protocol incorrectly listed storage under refrigerated conditions in a desiccator. During the study, the sodium 2,2-dimethylbutyrate neat standard was stored under frozen conditions (−20° C.) in a desiccator. This deviation had no impact on the study. Several weighings of Sodium 2,2-Dimethylbutyrate were previously made for stock solution stability analyses. The Sodium 2,2-Dimethylbutyrate stock solutions were found to be stable for 3.5 months, therefore, the neat standard must also be stable under frozen storage conditions.

For the specificity experiment, sodium 2,2-dimethylbutyrate was present in some controls at levels greater than 20% of the LLOQ level. This was a protocol deviation which specified that levels of sodium 2,2-dimethylbutyrate in plasma controls should be less than 20% of the LLOQ. This deviation had little effect on the study since the sodium 2,2-dimethylbutyrate background levels were at a low enough level that it did not interfere with the calibration curve and QCs.

The method presented here for the determination of sodium 2,2-dimethylbutyrate in rat plasma shows acceptable linearity, precision and accuracy for the calibration range of

0.2 μg/mL to 50 μg/mL. The method is specific for the internal standard, DMV, but did not meet the specificity criteria for sodium 2,2-dimethylbutyrate, since sodium 2,2-dimethylbutyrate was detected in blank plasma at a level up to 37% of the LLOQ concentration. It is believed that the sodium 2,2-dimethylbutyrate levels found in blank plasma are not related to the plasma, but can be considered background levels inherent in the method. The rat plasma can be diluted 10-fold and analyzed with acceptable precision and accuracy. At concentration levels within the calibration range, sodium 2,2-dimethylbutyrate in rat plasma is stable at room temperature on the bench top for at least 12.5 hours, and for three freeze/thaw cycles at approximately −70° C. Sodium 2,2-dimethylbutyrate is stable in rat plasma for at least 98 days when stored at approximately −70° C. The recovery in rat plasma ranged from 46.5% to 72.2% for sodium 2,2-dimethylbutyrate, and 50.2% to 67.6% for DMV. These are approximate recovery ranges since the CV % values were high due to the variability introduced by the derivatization step.

TABLE 20
System Suitability

| | ST-20 | DMV |
|---|---|---|
| Extraction | Peak Area | Peak Area |
| First | 33830.9 | 100329.1 |
| | 40215.0 | 106004.6 |
| | 42322.7 | 121702.0 |
| | 40678.2 | 120013.0 |
| | 45122.1 | 124658.7 |
| | 45193.4 | 127081.2 |
| Mean | 41227.1 | 116631.4 |
| SD | 4198.4 | 10858.0 |
| CV % | 10.2 | 9.3 |
| Second | 38539.8 | 106381.8 |
| | 42674.3 | 116091.2 |
| | 47207.1 | 130308.6 |
| | 48393.9 | 133137.5 |
| | 48290.6 | 131881.9 |
| | 47979.9 | 136795.3 |
| Mean | 45514.3 | 125766.1 |
| SD | 4041.1 | 11860.6 |
| CV % | 8.9 | 9.4 |
| Third | 8092.2 | 33024.4 |
| | 7693.9 | 32627.3 |
| | 6679.5 | 28932.7 |
| | 6326.1 | 26618.9 |
| | 6453.9 | 26311.3 |
| | 6634.5 | 26354.6 |
| Mean | 6980.0 | 28978.2 |
| SD | 729.5 | 3138.5 |
| CV % | 10.5 | 10.8 |
| Fourth | 12124.4 | 39116.7 |
| | 11695.9 | 37940.0 |
| | 11738.8 | 39760.6 |
| | 12455.5 | 37981.8 |
| | 11178.2 | 38984.5 |
| | 11887.8 | 38201.4 |
| Mean | 11846.8 | 38664.2 |
| SD | 431.4 | 736.7 |
| CV % | 3.6 | 1.9 |

TABLE 21
Standard curve parameters for 2,2-dimethylbutyrate.

| | | | | Correlation |
|---|---|---|---|---|
| Extraction | n | Intercept | Slope | Coefficient |
| First | 6 | 0.0187 | 0.309 | 0.9914 |
| Second | 7 | 0.0176 | 0.289 | 0.9988 |
| Third | 6 | 0.0407 | 0.184 | 0.9964 |
| Fourth | 7 | 0.0252 | 0.192 | 0.9984 |

Weighted (1/x²)

TABLE 22
Accuracy and Precision of 2,2-dimethylbutyrate
in QC Samples for Rat Plasma

| | LLOQ | QC-Low | QC-Mid | QC-High |
|---|---|---|---|---|
| Concentration (μg/mL) | 0.20 | 0.60 | 10.0 | 40.0 |
| | 0.201 | 0.640 | 9.88 | 41.7 |
| | 0.201 | 0.641 | 9.51 | 45.2 |
| | 0.199 | 0.638 | 9.56 | 44.1 |
| | 0.174 | 0.615 | 11.0 | 41.3 |
| | 0.197 | 0.655 | 11.3 | 42.1 |
| Mean | 0.194 | 0.638 | 10.2 | 42.9 |
| SD | 0.012 | 0.014 | 0.8 | 1.7 |
| CV % | 5.9 | 2.3 | 8.0 | 3.9 |
| RE % | −2.8 | 6.3 | 2.4 | 7.2 |

TABLE 23
Recovery of 2,2-dimethylbutyrate and DMV
from Rat Plasma at the Two QC Levels

| | Peak Area | | | |
|---|---|---|---|---|
| | QC-Low | | QC-High | |
| | 2,2- | DMV | 2,2- | DMV |
| Plasma QC | 15820.2 | 73174.4 | 1023189.0 | 79473.5 |
| | 12766.4 | 58975.0 | 661703.9 | 47418.6 |
| | 13594.8 | 63094.5 | 578799.8 | 42450.9 |
| | 11116.1 | 53350.5 | 544581.0 | 42664.2 |
| | 12905.7 | 58459.8 | 546420.5 | 42021.6 |
| Mean | 13240.6 | 61410.8 | 670938.8 | 50805.8 |
| SD | 1705.2 | 7430.1 | 202562.4 | 16175.3 |
| CV % | 12.9 | 12.1 | 30.2 | 31.8 |
| Fortified Water | 15983.9 | 70147.3 | 1604385.2 | 114286.7 |
| Blank | 21319.3 | 90370.4 | 1468135.4 | 99815.2 |
| | 26784.0 | 111857.4 | 1257197.9 | 89351.4 |
| Mean | 21362.4 | 90791.7 | 1443239.5 | 101151.1 |
| SD | 5400.2 | 20858.2 | 174927.4 | 12521.2 |
| CV % | 25.3 | 23.0 | 12.1 | 12.4 |
| Recovery % | 62.0 | 67.6 | 46.5 | 50.2 |

TABLE 24
Freeze/Thaw Cycle Stability at approximately −70°
C. for 2,2-dimethylbutyrate in Rat Plasma

2,2-DIMETHYLBUTYRATE

| | QC-Low | QC-High |
|---|---|---|
| Concentration (μg/mL) | 0.60 | 40.0 |
| | 0.595 | 39.2 |
| | 0.640 | 42.9 |
| | 0.491 | 41.7 |
| Mean | 0.575 | 41.2 |
| SD | 0.076 | 1.9 |
| CV % | 13.3 | 4.6 |
| RE % | −4.1 | 3.1 |

TABLE 25
Bench Top Stability for 2,2-dimethylbutyrate in Rat Plasma

2,2-DIMETHYLBUTYRATE

| | QC-Low | QC-High |
|---|---|---|
| Concentration (μg/mL) | 0.60 | 40.0 |
| | 0.600 | 45.6 |
| | 0.677 | 37.9 |
| | 0.660 | 47.4 |
| Mean | 0.646 | 43.6 |
| SD | 0.040 | 5.1 |
| CV % | 6.3 | 11.7 |
| RE % | 7.6 | 9.1 |

TABLE 26
Long-Term Freezer Stability at approximately −70°
C. for 2,2-dimethylbutyrate in Rat Plasma

2,2-dimethylbutyrate

| | | |
|---|---|---|
| | QC-Low | QC-High |
| Concentration (μg/mL) | 0.60 | 40.0 |

1 Month Interval

| | | |
|---|---|---|
| | 0.473 | 34.4 |
| | 0.410 | 35.4 |
| | 0.484 | 34.3 |
| Mean | 0.456 | 34.7 |
| SD | 0.040 | 0.6 |
| CV % | 8.8 | 1.7 |
| RE % | −24.1 | −13.3 |

3 Month Interval

| | | |
|---|---|---|
| | 0.663 | 38.0 |
| | 0.605 | 38.0 |
| | 0.605 | 35.5 |
| Mean | 0.62 | 37.2 |
| SD | 0.03 | 1.4 |
| CV % | 5.4 | 3.9 |
| RE % | 4.1 | −7.1 |

TABLE 27
10-Fold Dilution QC Samples for 2,2-
dimethylbutyrate in Rat Plasma

| | 2,2- |
|---|---|
| Concentration | 100 |
| | 106 |
| | 95.6 |
| | 97.2 |
| Mean | 99.5 |
| SD | 5.4 |
| CV % | 5.4 |
| RE % | −0.5 |

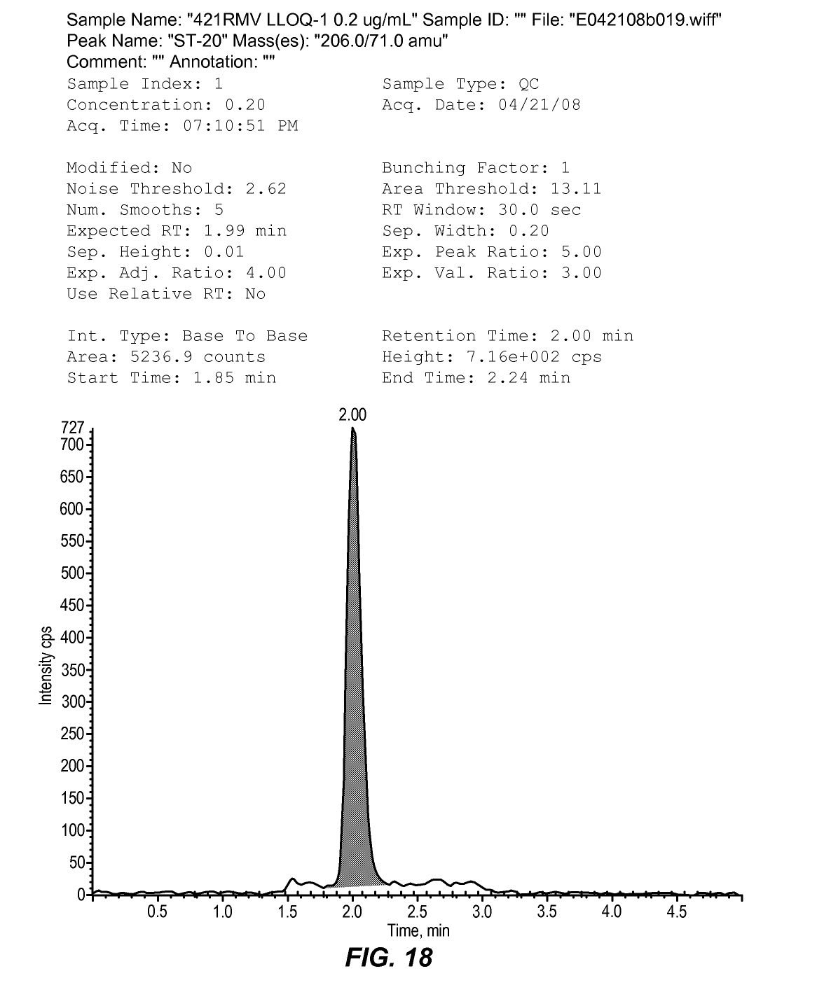

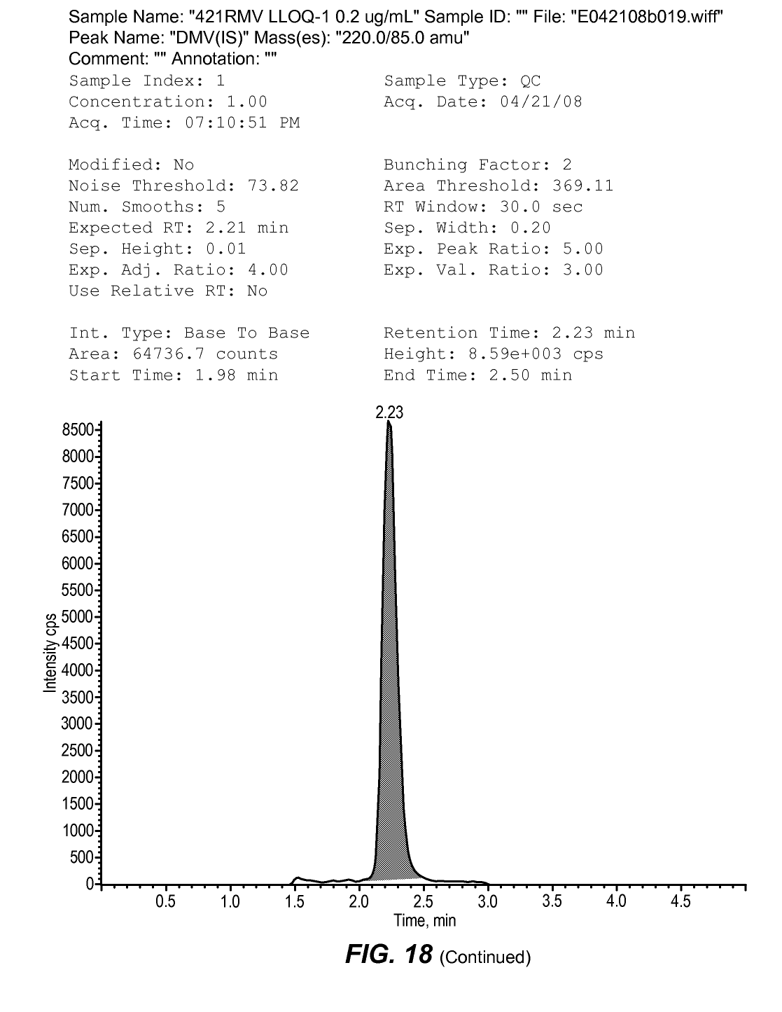

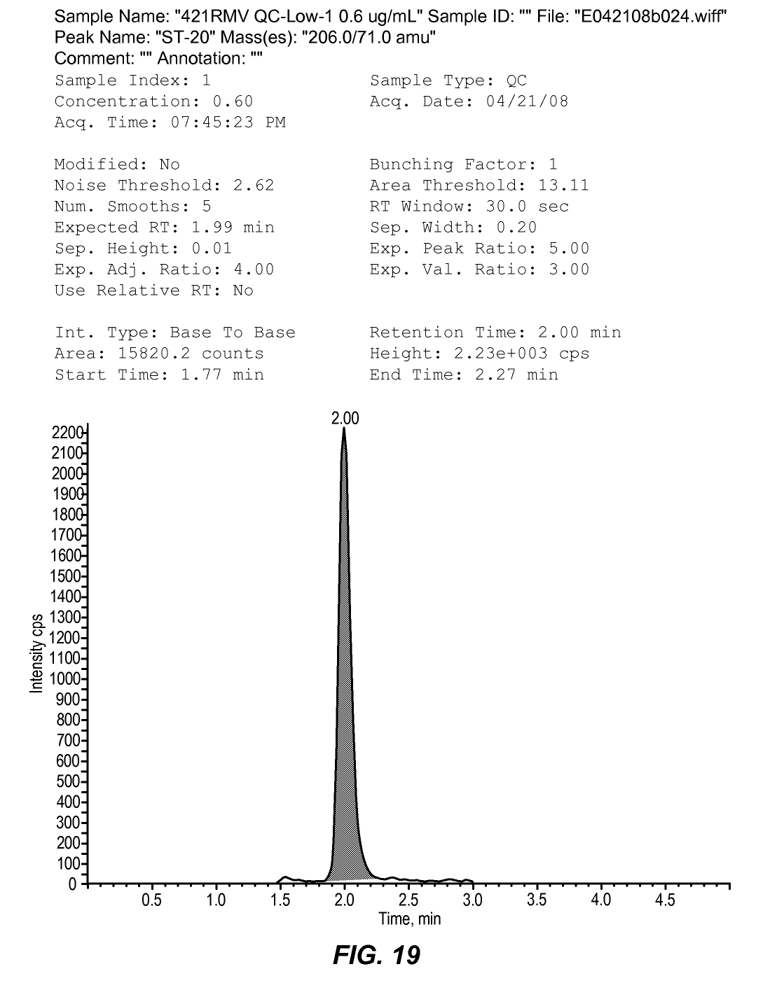

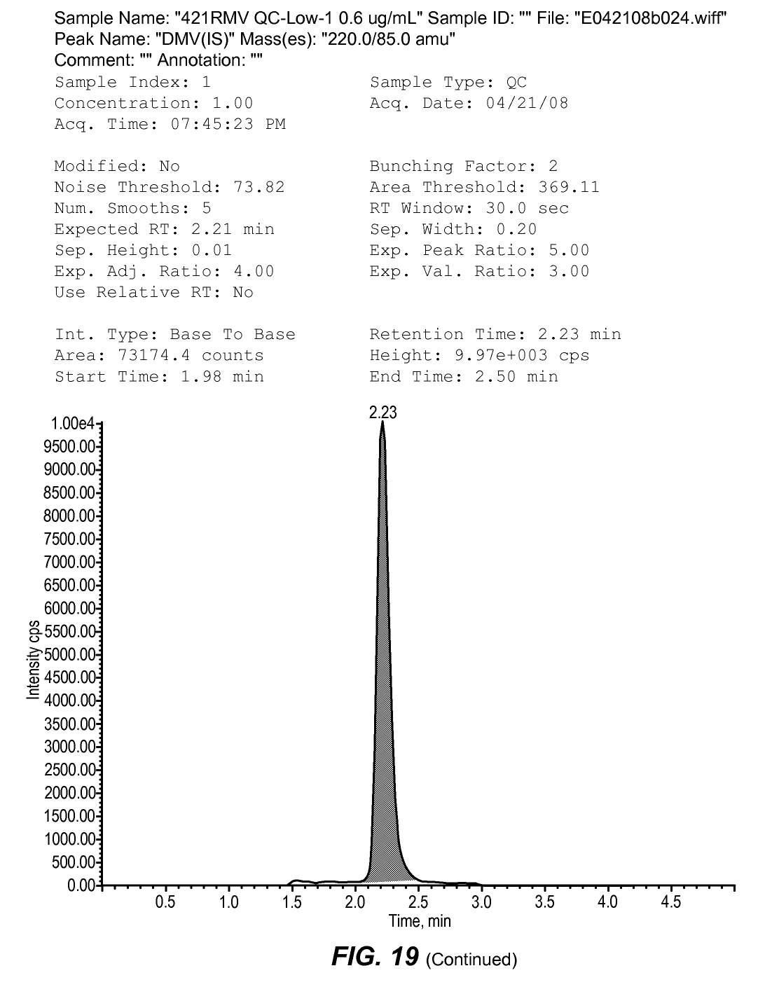

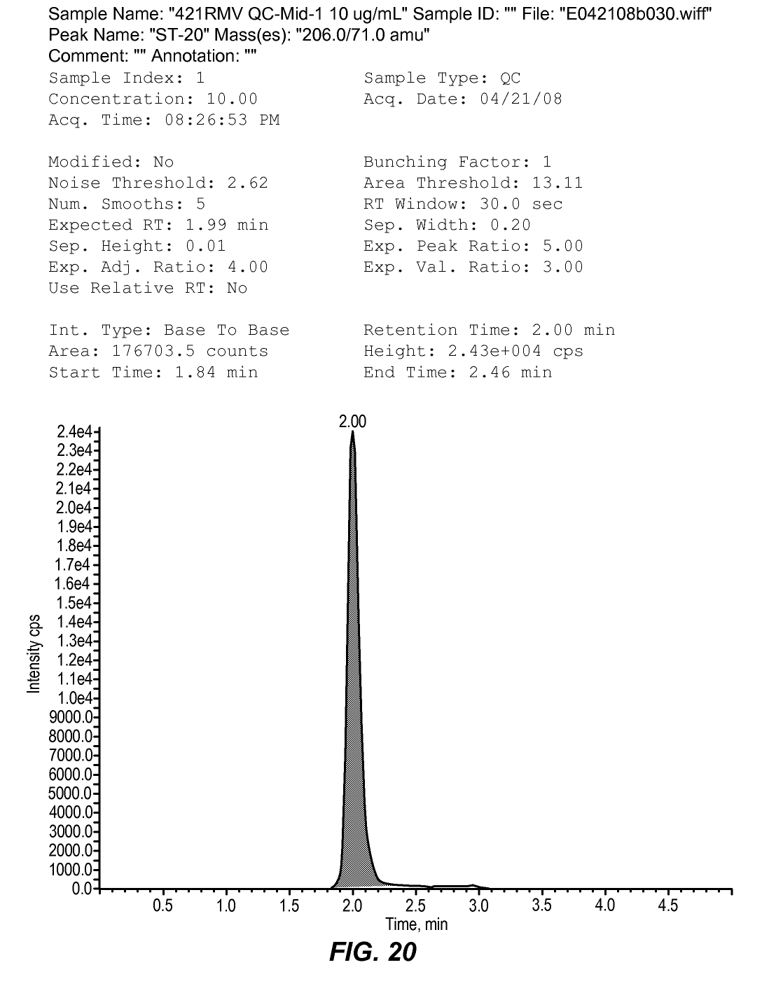

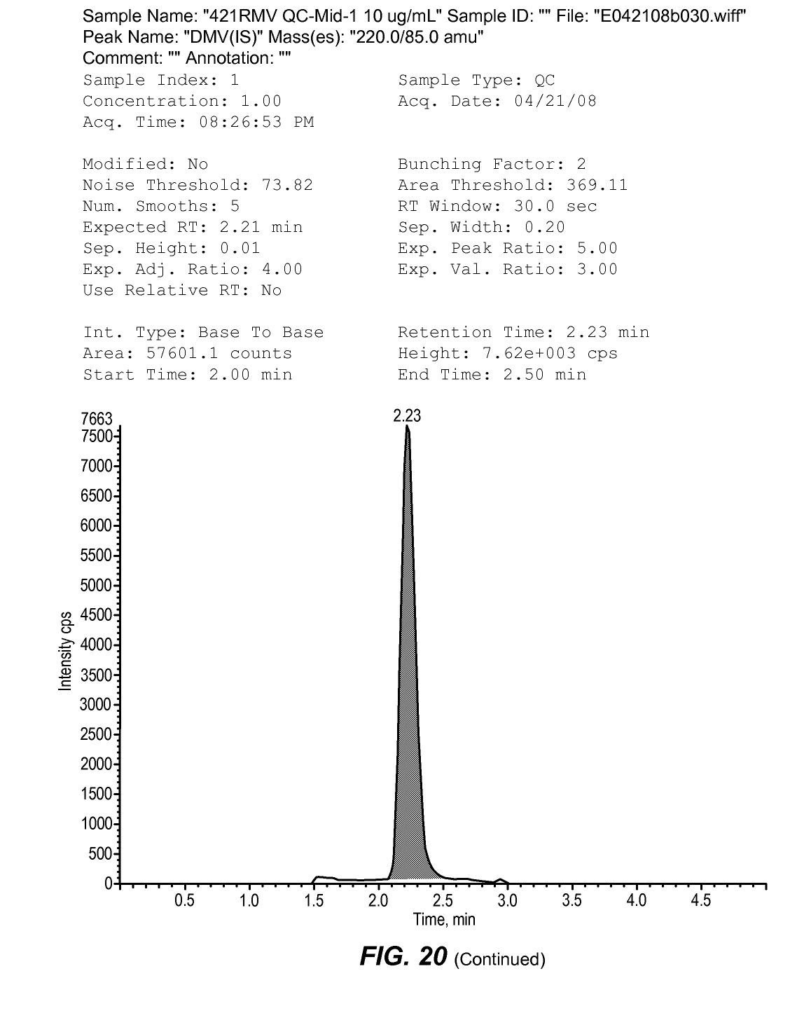

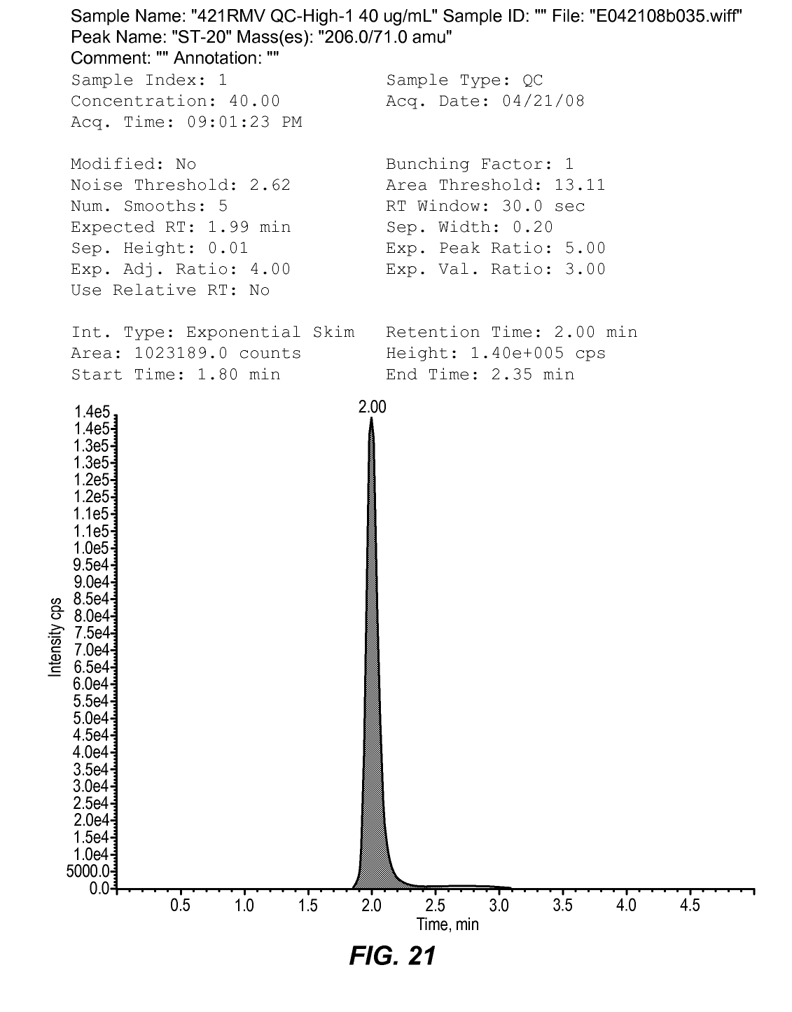

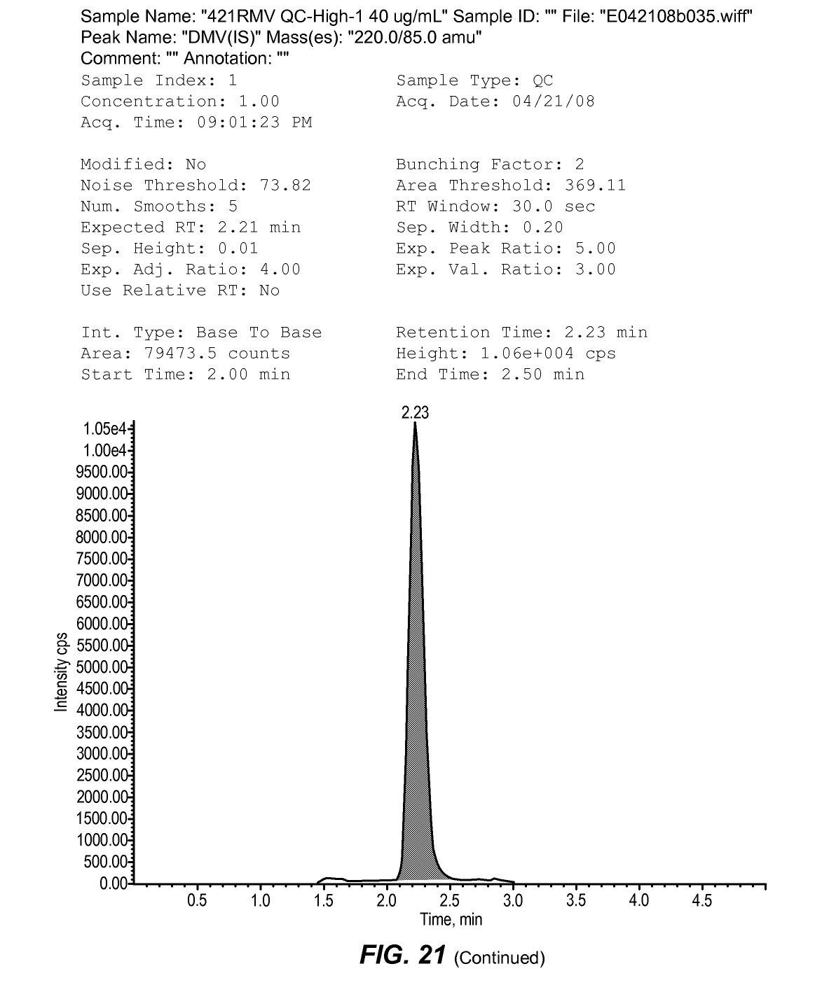

Representative chromatograms from the study are illustrated in FIGS. 15-21. FIG. 15 illustrates representative Calibration Curve. FIG. 16 illustrates representative Chromatograms of Control Human Plasma sodium 2,2-dimethylbutyrate (top), DMV (bottom). FIG. 17 illustrates representative Chromatograms of Standard-1 (0.2 μg/mL Sodium 2,2-Dimethylbutyrate) in Human Plasma sodium 2,2-dimethylbutyrate (top), DMV (bottom). FIG. 18 illustrates representative Chromatograms of LLOQ (0.2 ug/mL sodium 2,2-dimethylbutyrate) in Human Plasma sodium 2,2-dimethylbutyrate (top), DMV (bottom). FIG. 19 illustrates representative Chromatograms of Low QC (0.6 ug/mL sodium 2,2-dimethylbutyrate) in Human Plasma sodium 2,2-dimethylbutyrate (top), DMV (bottom). FIG. 20 illustrates representative Chromatograms of QC-Mid (10 ug/mL sodium 2,2-dimethylbutyrate) in Human Plasma Sodium 2,2-Dimethylbutyrate (top), DMV (bottom). FIG. 21 illustrates representative Chromatograms of QC-High (40 ug/mL sodium 2,2-dimethylbutyrate) in Human Plasma sodium 2,2-dimethylbutyrate (top), DMV (bottom).

### Example 4

### Examination of DMB Levels in Human Plasma and Urine Using SPE and LC-MS/MS

Samples of human plasma and urine were tested for the presence of 2,2-dimethylbutyrate (DMB). Plasma or urine samples were fortified with an internal standard (DMV) and loaded onto an SPE cartridge (Waters OASIS™, HLB 30 mg, 1 ml cartridge) for extraction of the analyte. After washing and drying down to remove water, 2,2-dimethylbutyric acid was eluted from the SPE cartridge, followed by a derivatization reaction using Deoxo-Fluor as derivatization agents. After derivatization, 5-10 ul of reconstitution solution were injected to LC-MS/MS. The column used was the Unison UK-C18 3μ, 30×2. Mobile phase A (0.1% formic acid in water) and Mobile phase B (0.1% formic acid in methanol/water (98/2, v/v) were used. Needle wash was performed with Mobile phase B and elution was performed using gradient program and a run time of 4.6 minutes. Analysis was performed using a Shmadzu HPLC system coupled to an API 4000 Q Trap mass spectrometer, which was operated in turbo ionspray in positive ion MRM mode.

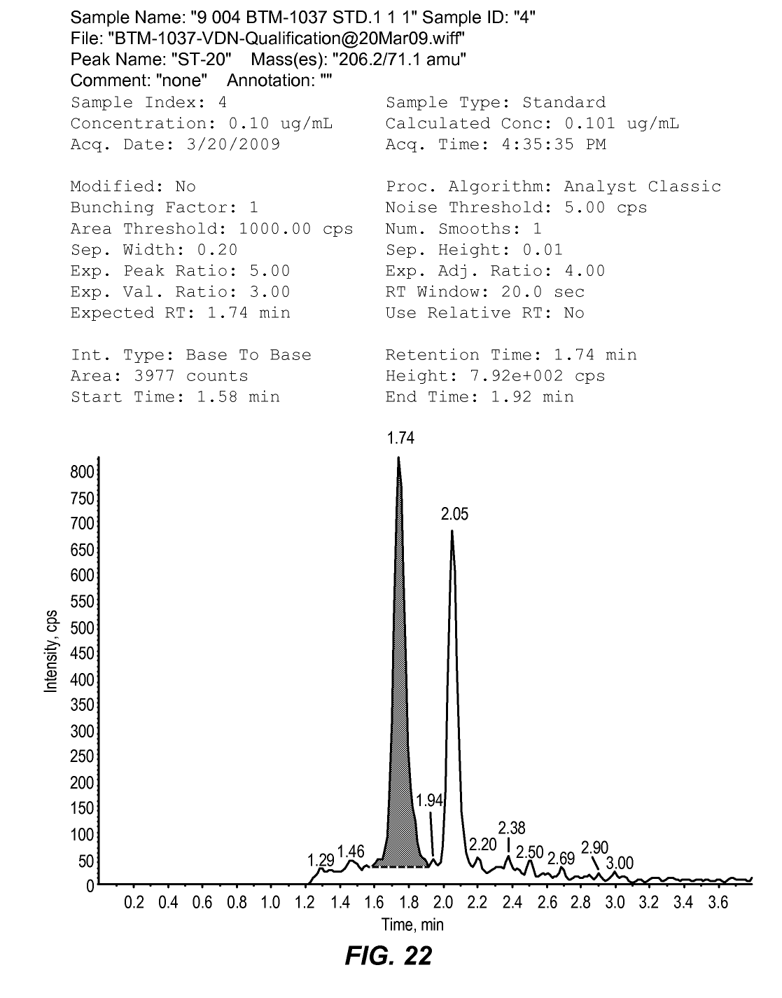

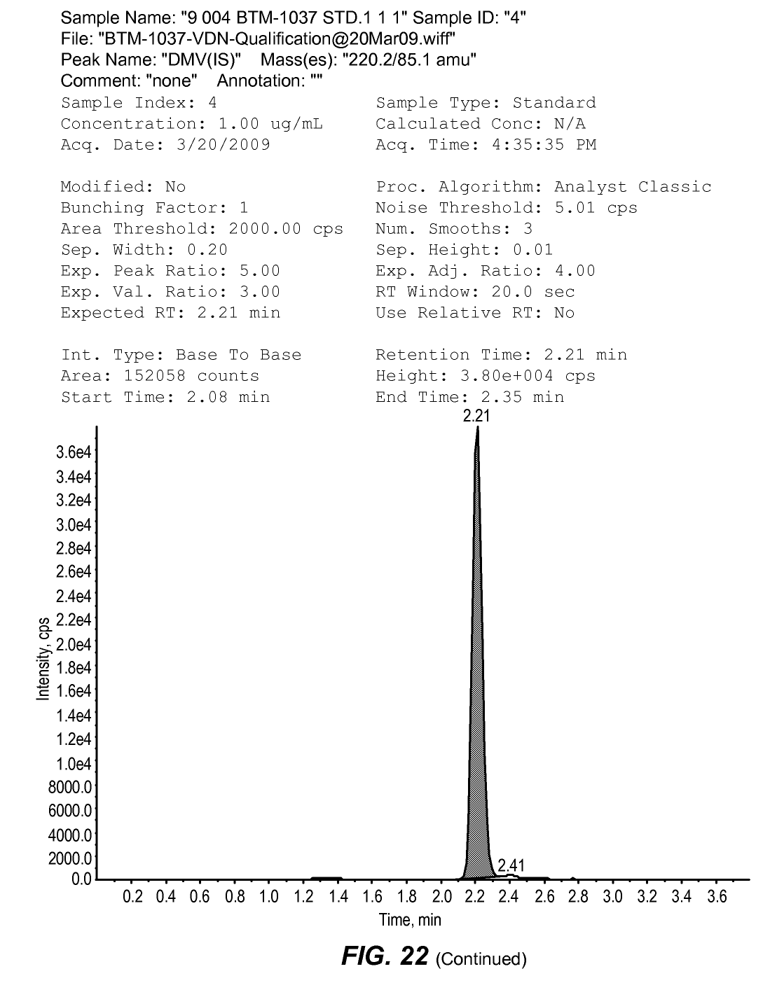

The transitions (precursor to daughter) monitored were m/z 206.0>71.0 m/z for 2,2-dimethylbutyric acid and 220.0>85.0 m/z for dimethylvaleric acid (IS). Separation of the analytes was achieved on a Phenomenex Synergi column (150×2 mm, 4 um) with a gradient elution. A representative result (LLOQ) is shown in FIG. 22. More than 2000 samples were tested to determine whether the test could be used as a monitor for therapeutic effectiveness. Precision and accuracy for human plasma samples are shown in Table 28 and for human urine samples in Table 29.

TABLE 28
Intrarun Precision and Accuracy for Human Plasma Samples

| Sample | % CV | % Nominal | Number |
|---|---|---|---|
| LLOQ | 14.4 | 104.0 | 6 |
| QC Low | 5.9 | 107.3 | 6 |
| QC Mid | 1.7 | 102.7 | 6 |
| QC high | 5.6 | 92.5 | 6 |

TABLE 29
Intrarun Precision and Accuracy for Human Urine Samples

| Sample | % CV | % Nominal | Number |
|---|---|---|---|
| QC Low | 5.6 | 97.2 | 17 |
| QC Mid | 3.6 | 100.3 | 17 |
| QC high | 4.8 | 100.0 | 17 |

### Example 5

### Determination of Sodium 2,2-Dimethylbutyrate in Human Urine by LC/MS/MS

Next, it was determined whether sodium 2,2-dimethylbutyrate (DMB) could be detected in human urine using the solid-phase methods described herein. Dimethylvaleric acid (DMV) was used as an internal standard. Sodium 2,2-dimethylbutyrate and DMV were extracted from samples by solid-phase extraction from human urine followed by chemical derivatization. Reversed-phase HPLC separation was achieved with a Unison UK-C18 column. Two MS/MS approaches were used. In the first approach (method I), MS/MS detection was set at a mass transitions of 206.0 to 71.0 m/z for sodium 2,2-dimethylbutyrate and 220.0 to 85.0 for DMV in electron spray ionization positive mode. In the second approach (method II), MS/MS detection was set at mass transitions of 202.6 to 71.1 m/z for sodium 2,2-dimethylbutyrate and 220.2 to 85.1 m/z for DMV in turboionspray positive mode. For LC/MS/MS, a sciex API 4000 (QTRAP) with Shimadzu HPLC pump and auto sampler were used with a Phenomenex Synergi 4μ, Hydro RP, 150×2 mm column and an OASIS HLB SPE cartridge (30 μm, 1 cc-10 mg). Human urine (six lots, tested individually or pooled), purchased from Bioreclamation Inc. was used in these analyses.

Stock solutions of sodium 2,2-dimethylbutyrate were found to be stable at a nominal temperature of 4° C. for at least 115 days. The quality control sample of sodium 2,2-dimethylbutyrate in human urine were found to be stable at a storage temperature of −20° C. for at least 111 days. In method I, the mobile phase of liquid chromatography was run on 0.1% formic acid in acetonitrile, and the elution volume was 10 ul. In method II, the mobile phase was run in 0.1% formic acid in methanol/water (98/2, volume to volume), and the elution volume was 5 ul.

The calibration standards were prepared by mixing sodium 2,2-dimethylbutyrate with blank human urine sample. The following concentrations of sodium 2,2-dimethylbutyrate were used: 0.1 ug/ml, 0.2 ug/ml, 1 ug/ml, 10 ug/ml, 20 ug/ml, 30 ug/ml, 40 ug/ml, and 50 ug/ml. Concentrations of sodium 2,2-dimethylbutyrate in quality control sample were as follows: QC-low, 0.3 ug/ml; QC-mid, 19 ug/ml; QC-high, 38 ug/ml. Concentrations were calculated using linear regression according to the following equation: y=ax+b where y equals to peak area ratio of analyte/internal standard; a equals to slope of the corresponding standard curve; x equals to concentration of analyte in ug/ml; b equals to intercept of the corresponding standard curve. 1/x² was used as weighting factor. For calculation of accuracy the following equation was used: % of nominal=mean measured concentration/nominal concentration. For calculation of precision, the following equation was used: % of coefficient of variation=standard deviation/mean measured concentration.

Six different lots of blank human urine was tested either with or without internal standard for selectivity of the methods in which the ability of selected chromatographic method is measured whether a response form the analyte can be determined without interference from biological matrix. No significant baseline interference was observed in method I and method II. Sensitivity was determined with Lower quantitation limit target as 0.1 ug/ml of sodium 2,2-dimethylbutyrate. 6 samples at LLOQ limit were analyzed and the concentrations were calculated with the calibration curve. Both methods demonstrated sufficient sensitivity to detect 0.1 ug/ml of sodium 2,2-dimethylbutyrate with a mean value 0.110 with a standard deviation 0.018. % coefficient of variation (CV) was less than 20%.

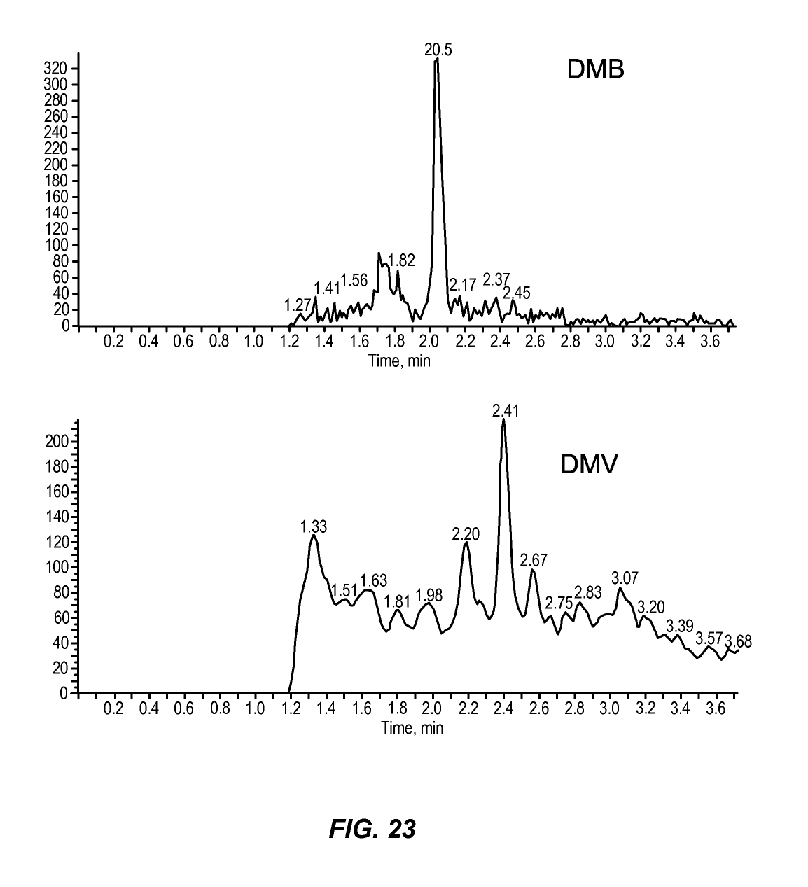

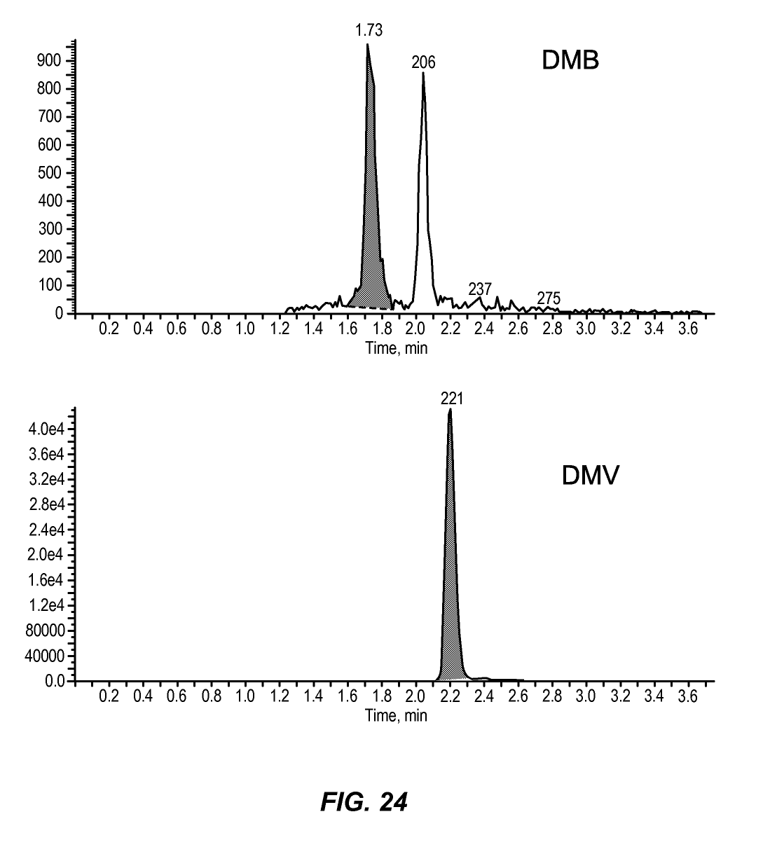

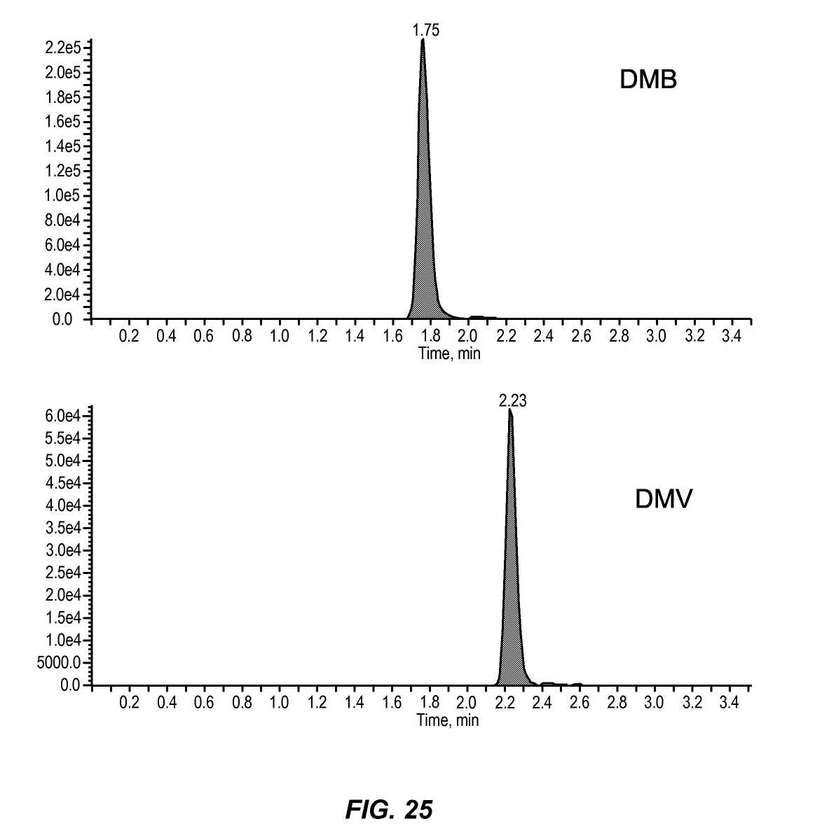

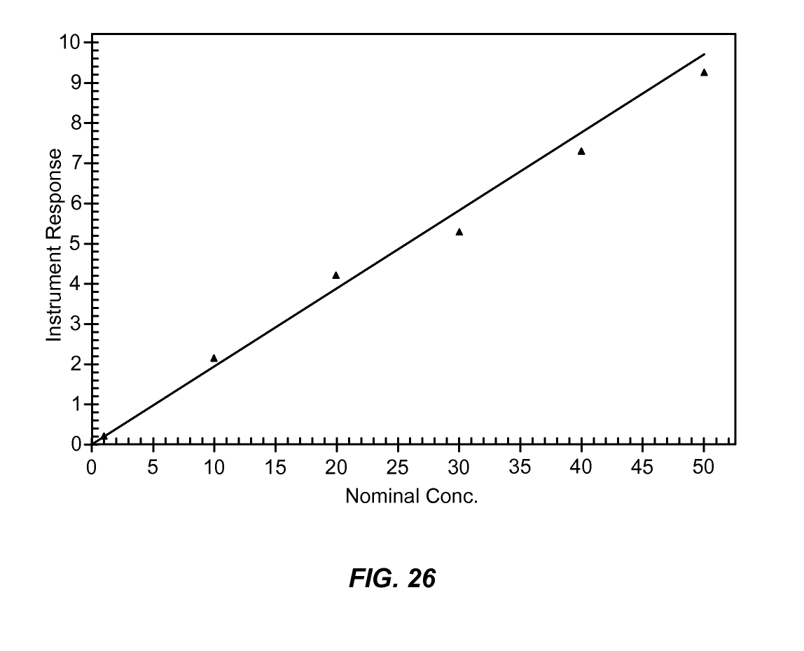

Back-calculated concentrations of calibration standards did not differ by more than 15% from the nominal concentration (Table 30). Six (6) samples with a concentration of 0.3 ug/ml, 19 ug/ml and 38 ug/ml were used to measure intraday accuracy and precision. The accuracy was within 100±15% and % CV was less than 15% (Table 31). Also, results of dilution assay, when tested from at a concentration 2 times above the upper limit of quantitation (100 ug/ul) with six replicate demonstrated that the dilution integrity was within 100±15% and % CV was less than 15%. Thus, both method I and method II were suitable for the determination of DMB in human urine. Representative chromatograms of the validation study are shown in FIGS. 23-25. Representative calibration curve from the study is shown in FIG. 26. Additionally, recovery of DMB from stock solutions was performed at three concentration levels using the methods described. At a concentration of 0.3 μg/ml, 92.3% of DMB was recovered, at a concentration of 19 μg/ml, 99.9% of DMB was recovered and at a concentration of 38 μg/ml, 68.4% of DMB was recovered (overall 86.9% recovery).

TABLE 30
Back-calculated Concentrations of DMB

| Run # | 0.1 ug/ml | 0.2 ug/ml | 1 ug/ml | 10 ug/ml | 20 ug/ml | 30 ug/ml | 40 ug/ml | 50 ug/ml |
|---|---|---|---|---|---|---|---|---|
| 1 | 0.098 | 0.205 | 1.052 | 10.610 | 20.274 | 29.580 | 39.092 | 45.100 |
| 2 | 0.102 | 0.192 | 0.974 | 9.742 | 21.746 | 30.009 | 37.990 | 51.399 |
| 3 | 0.101 | 0.193 | 1.029 | 10.381 | 20.806 | 31.899 | 38.277 | 44.614 |
| 4 | 0.099 | 0.200 | 1.077 | 10.086 | 21.447 | 26.791 | 39.381 | 48.557 |
| 5 | 0.101 | 0.198 | 1.003 | 9.993 | 21.369 | 31.129 | 39.618 | 45.393 |
| 6 | 0.099 | 0.204 | 1.003 | 10.326 | 21.460 | 29.794 | 40.196 | 44.253 |
| 7 | 0.102 | 0.192 | 0.987 | 10.681 | 21.090 | 29.422 | 38.273 | 48.563 |
| Mean | 0.100 | 0.198 | 1.018 | 10.260 | 21.170 | 29.816 | 38.975 | 46.840 |
| % Nominal | 100.3 | 98.9 | 100.8 | 102.6 | 105.9 | 99.4 | 97.4 | 93.7 |
| % CV | 1.6 | 2.8 | 3.6 | 3.3 | 2.3 | 5.4 | 2.1 | 5.7 |

TABLE 31
Intraday and Interday Accuracy and Precision

| | | DMB, | DMB, | DMB, |
|---|---|---|---|---|
| Day ID | Sample No. | 0.3 μg/ml | 19 μg/ml | 38 μg/ml |
| Intraday 1 | 1 | 0.323 | 19.217 | 37.258 |
| | 2 | 0.317 | 19.507 | 36.788 |
| | 3 | 0.287 | 20.293 | 36.268 |
| | 4 | 0.330 | 20.571 | 38.644 |
| | 5 | 0.327 | 21.036 | 37.738 |
| | 6 | 0.337 | 21.414 | 36.655 |
| | Mean | 0.320 | 20.340 | 37.225 |
| | % Nominal | 106.7 | 107.1 | 98.0 |
| | % CV | 5.5 | 4.2 | 2.3 |
| Intraday 2 | 1 | 0.314 | 19.601 | 40.145 |
| | 2 | 0.255 | 21.350 | 34.735 |
| | 3 | 0.271 | 19.116 | 39.554 |
| | 4 | 0.300 | 21.901 | 36.707 |
| | 5 | 0.303 | 18.662 | 36.026 |
| | 6 | 0.261 | 20.757 | 34.296 |
| | Mean | 0.284 | 20.231 | 36.911 |
| | % Nominal | 94.7 | 106.5 | 97.1 |
| | % CV | 8.7 | 6.4 | 6.6 |
| Intraday 3 | 1 | 0.311 | 18.062 | 36.675 |
| | 2 | 0.329 | 16.348 | 33.941 |
| | 3 | 0.280 | 16.609 | 34.712 |
| | 4 | 0.331 | 19.228 | 33.389 |
| | 5 | 0.264 | 18.305 | 36.400 |
| | 6 | 0.270 | 18.584 | 36.891 |
| | Mean | 0.298 | 17.856 | 35.335 |
| | % Nominal | 99.2 | 94.0 | 93.0 |
| | % CV | 10.1 | 6.4 | 4.3 |
| Interday | Mean | 0.301 | 19.476 | 36.490 |
| | % Nominal | 100.2 | 102.5 | 96.0 |
| | % CV | 9.2 | 8.1 | 5.0 |

The applicability of the testing methodology to analyze samples stored under different conditions was analyzed. First, to assess short-term stability of DMB in human urine, samples at 0.3, 19 and 38 μg/ml, 92.3% of DMB were maintained unextracted at room temperature and at bench-top light levels for six hours. Assays on the samples were performed as above and indicated that DMB is stable under these conditions and concentrations (% CV no more than 15%). Second, to assess freeze/thaw stability of DMB in human urine, samples at 0.3, 19 and 38 μg/ml, 92.3% of DMB were stored at −70° C. and thawed. This process was repeated twice and samples after each freezing were analyzed for DMB as described. For each concentration and each freezing/thawing sample, the % CV was no more than 15%. These results demonstrate that analysis of DMB-containing urine samples can be analyzed after samples are contained in such conditions.

These results show that the disclosed methodology can be utilized to determine physiological levels of short-chain fatty acids (e.g., 2,2-dimethylbutyric acid). Such tests can allow for determination and/or modification of dosage levels of DMB for patients to achieve and/or maintain physiologically effective concentrations of such compounds.

### Example 6

### Alteration of a Therapeutic Regimen by Measuring Plasma Levels of DMB in a Patient

In this example, the analytical devices and methods described herein are used to analyze a patient's plasma level to monitor therapeutic regimen. A patient in need of therapy with DMB (e.g., a person with beta thalassemia) is dosed with 50 mg of DMB orally three times in a single day. Blood samples are taken at one hour, four hours and six hours following the first dose and four hours following the second and third doses. Blood samples are also taken 24 hours following the final dose. Concentrations of DMB in each sample are determined to ensure that the patient achieves a therapeutic concentration and that the therapeutic concentration is maintained for at least 24 hours following the final dose. The patient is also monitored for improvements in clinical manifestations (e.g., lessened pain). Where analyses show the patient is not achieving and/or is not maintaining a therapeutically effective plasma concentration (e.g., lower than 200 μM), dosage can be increased. Alternately, where analyses show the patient in achieving and/or maintaining a plasma concentration above 1000 μM, dosage can be decreased. In some instances, determination of an increase, decrease or maintenance of expression levels of fetal globin (which is induced by DMB) can also be determined. Such additional data can be examined along with plasma (or blood or urine) concentration of DMB to determine whether a dosage regimen should be altered in the patient.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for detecting or quantifying a short-chain fatty acid in a biological sample from a subject, comprising:
a) purifying the short-chain fatty acid by removing at least a portion of non-short-chain fatty acid components of the sample, wherein the purifying step comprises subjecting the sample to solid phase extraction;
b) chemically derivatizing said purified short-chain fatty acid;
c) subjecting said derivatized product to mass spectrometry; and
d) determining the presence or quantity of the derivatized product, thereby detecting or quantifying said short-chain fatty acid in said sample.

2. The method of claim 1, wherein said short-chain fatty acid is butyric acid or a derivative of butyric acid.

3. The method of claim 2, wherein said derivative of butyric acid is 2,2-dimethylbutyric acid.

4. The method of claim 1, wherein said subject is a human.

5. The method of claim 1, wherein said subject has received a therapeutic dose of 2,2-dimethylbutyric acid or a pharmaceutically acceptable salt thereof.

6. The method of claim 1, wherein said biological sample is a blood or urine sample.

7. The method of claim 1, wherein the chemical derivatizing step comprises the use of a fluorinating agent, an aromatic amine, or both.

8. The method of claim 1, further comprising the step of reconstituting said derivatized product prior to subjecting said derivatized product to mass spectrometry.

9. The method of claim 8, wherein said reconstituting step comprises exposing said derivatized product to a mixture of water and acetonitrile.

10. The method of claim 9, wherein said water and acetonitrile are at a ratio of at least 75/25 v/v.

11. The method of claim 1, wherein said short-chain fatty acid is a therapeutic short-chain fatty acid.

12. A method of monitoring treatment in a subject receiving a therapeutic short-chain fatty acid, comprising:
a) purifying a short-chain fatty acid from said subject, wherein said purifying comprises subjecting said sample to solid phase extraction;
b) chemically derivatizing said purified short-chain fatty acid;
c) subjecting said derivatized product to mass spectrometry;
d) determining the presence or quantity of the derivatized product, thereby quantifying said therapeutic short-chain fatty acid in said sample; and
e) using the data collected from step d) to make a clinical decision.

13. The method of claim 12, wherein said therapeutic short-chain fatty acid is butyric acid or a butyric acid derivative, or a pharmaceutically acceptable salt or ester thereof.

14. The method of claim 13, wherein said butyric acid derivative is 2,2-dimethylbutyrate or a pharmaceutically acceptable salt or ester thereof.

15. The method of claim 12, wherein said subject is a human.

16. The method of claim 12, wherein said subject has, or is at risk of developing, a blood disorder.

17. The method of claim 16, wherein said blood disorder is sickle cell anemia or beta thalassemia.

18. The method of claim 12, wherein said subject has or is at risk of developing a cell proliferative disorder.

19. The method of claim 19, wherein said cell proliferative disorder is cancer or cytopenia.

20. The method of claim 12, further comprising the step of reconstituting said derivatized product prior to subjecting said derivatized product to mass spectrometry.

21. The method of claim 12, wherein said subject has, or is at risk of developing, a viral related malignancy.

22. The method of claim 12, wherein said subject has, or is at risk of developing, a viral related proliferative disorder.

23. The method of claim 12, wherein said subject has, or is at risk of developing, an inflammatory disorder.

24. The method of claim 12, wherein said subject has, or is at risk of developing, an autoimmune disease.

25. The method of claim 12, wherein said subject has, or is at risk of developing, atherosclerosis.
